(19) United States
(12) Patent Application Publication
(10) Pub. No.: US 2009/0047221 A1
(43) Pub. Date: Feb. 19, 2009

(54) COMPOUNDS AND COMPOSITIONS USEFUL IN THE TREATMENT OF NEOPLASIA

(75) Inventors: Allen McClay, Cookstown (GB); David Waugh, County Down (GB); Paul Armstrong, Belfast (GB); Zoica Delbederi, County Armagh (GB); Catherine Higgins, County Monaghan (IE); Hendrik Van Den Berg, Belfast (GB); Patrick Johnston, Belfast (GB); William Watters, County Antrim (GB); Kelly McGarel, Larne (GB); Timothy Mils, Belfast (GB)

Correspondence Address: PEPPER HAMILTON LLP, ONE MELLON CENTER, 50TH FLOOR, 500 GRANT STREET, PITTSBURGH, PA, 15219, US

(73) Assignee: NIPRI LIMITED, Belfast (GB)

(21) Appl. No.: 11/718,314

(22) PCT Filed: Oct. 31, 2004
(86) PCT No.: PCT/GB2005/004190

§ 371 Date: Apr. 30, 2007

(30) Foreign Application Priority Data
Oct. 29, 2004 (GB) GB 0424051.

(51) Int. Cl. A61K 9/68; C07D 405/10; A61K 31/4178; A61P 35/00; A61K 31/7048; C12N 5/06; C07D 311/76; G01N 33/566; A61K 31/437; A61K 31/4375; A61K 33/24; A61K 31/704

(52) U.S. Cl. 424/48; 548/311.4; 514/397; 514/27; 424/649; 514/34; 435/375; 436/501; 549/289

(57) ABSTRACT

There is described compounds for use in therapy, said compounds being defined by Formula (1): There is also described an anti-proliferative composition comprising one or more compounds according to Formula (1), and a method of treatment of neoplasia comprising the administration of such a compound or composition.

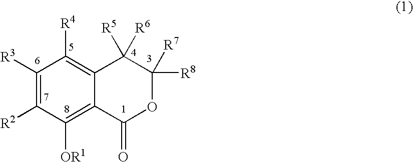

This invention relates to compounds and compositions for use in therapy, particularly but not exclusively to compounds and compositions for use in the treatment of neoplasia.

Neoplasms, which include cancers and other benign tumours, are a major cause of suffering and death in both humans and animals. Although some cancers are treatable, for example through radio therapeutic or chemotherapeutic techniques, many remain difficult or impossible to treat effectively.

Accordingly there is a long felt need for alternative or improved therapies. It is therefore desirable to identify improved or alternative therapies which may permit physicians to treat neoplasia more effectively.

According to a first aspect of the present invention there is provided compounds for use in therapy or diagnosis, said compounds being defined by Formula 1:

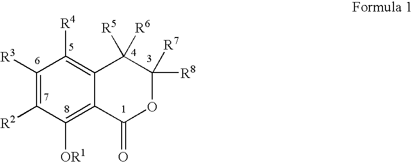

R¹ is an aliphatic or aromatic hydrocarbon group which may be substituted or unsubstituted. Suitably the hydrocarbon group is substituted with one or more of any substituted or unsubstituted alkane, alkene, alkyne or aromatic hydrocarbon groups.

R¹ may suitably contain one or more amine, amide, nitrile, halogen, ether, alcohol, thiol, acid (such as carboxylic or sulphonic or phosphoric acid), ester, aldehyde, ketone, phosphine or phosphine oxide groups.

In one embodiment R¹ represents H, a C₁₋₂₅ aliphatic or aromatic hydrocarbon group, CHO, COR⁹, CO₂R⁹, CONR₂⁹, SO₂R⁹, SO₃R⁹, PO(OR⁹)₂, PO(OR⁹)NR₂⁹, PO(NR₂⁹)₂.

Each R⁹ group is independently selected, and each compound according to Formula 1 may comprise more than one R⁹ group, wherein each R⁹ group may be the same or different. R⁹ represents H or an optionally substituted aliphatic or aromatic hydrocarbon group.

In one embodiment of the present invention each R⁹ group is independently selected from the group consisting of H, C_{1 to 25} alkyl, C_{1 to 25} alkenyl, C_{1 to 25} alkynyl, C_{6 to 14} aryl or C_{7 to 25} aralkyl.

In one embodiment the R⁹ group is unsubstituted.

Alternatively the R⁹ group may be substituted with one or more of any substituted or unsubstituted alkane, alkene, alkyne or aromatic hydrocarbon group. In one embodiment the R⁹ group is substituted with one or more of OH, OR¹⁰, OCOR¹⁰, NH₂, NR₂¹⁰, CN, NO₂, halogen, SO₃H, CHO, COR¹⁰, SH, SR¹⁰, CO₂H, CO₂R¹⁰, CONR₂¹⁰, SO₂NR₂¹⁰, PO(OR¹⁰)₂, PO(NR₂¹⁰)₂ and PO(OR¹⁰)NR₂¹⁰; wherein R¹⁰ represents H, or an optionally substituted C_{1 to 25} alkyl, C_{1 to 25} alkenyl, C_{1 to 25} alkynyl, C_{6 to 14} aryl or C_{7 to 25} aralkyl group. R¹⁰ may be substituted with one or more of OH, OR, OCOR, NH₂, NR₂, CN, NO₂, halogen, SO₃H, CHO, COR, SH, SR, CO₂H, CO₂R, CONR₂, SO₂NR₂, PO(OR)₂, PO(NR₂)₂ and PO(OR)NR₂; wherein R represents H, C_{1 to 25} alkyl C_{1 to 25} alkenyl, C_{1 to 25} alkynyl, C_{6 to 14} aryl or C_{7 to 25} aralkyl.

R² to R⁸ may be the same or different. R² to R⁸ independently represent substituted or unsubstituted aliphatic or aromatic hydrocarbon groups, H, OH, OR⁹, OCOR⁹, OSO₂R⁹, OPO(OR⁹)₂, OPO(OR⁹)NR₂⁹, OPO(NR₂⁹)₂, NH₂, NR₂⁹, COR⁹, SO₂R⁹, CN, NO₂, halogen, SO₃H, CHO, COR⁹, SH, SR⁹ SOR⁹, PO(OR⁹)₂, CO₂H, CO₂R⁹, CONR₂⁹, SO₂NR₂⁹ SO₃R⁹, PO(NR₂⁹)₂ PO(OR⁹) (NR₂⁹) or N₃ wherein R⁹ is as defined above.

In one embodiment R⁴ may not represent Cl.

The groups listed above for R² to R⁸ may be branched, linear, cyclic or non-cyclic.

In one embodiment of the invention there is a proviso that R⁵ and R⁶ and/or R⁷ and R⁸ are not simultaneously OH, SH or NH₂.

One or more of R² to R⁸ may suitably represent an aliphatic or aromatic hydrocarbon group substituted with one or more of OH, OR⁹, OCOR⁹, NH₂, NR₂⁹, CN, NO₂, halogen, SO₃H, CHO, COR⁹, SH, SR⁹, SOR⁹ CO₂H, CO₂R⁹, CONR₂⁹, SO₂NR₂⁹, PO(OR⁹)₂, PO(OR⁹) (NR₂⁹) or PO(NR₂⁹)₂ where R⁹ is as defined above.

R⁴ may not represent Cl.

Suitably R⁹ represents H, C_{1 to 25} alkyl, C_{1 to 25} alkenyl, C_{1 to 25} alkynyl, C_{6 to 14} aryl or C_{7 to 25} aralkyl.

In one embodiment R⁵ and R⁶ represent H and R⁷ and R⁸ represent CH₃ or H where either or both of R⁷ and R⁸ may represent CH₃, or either or both of R⁷ and R⁸ may represent H.

In a further embodiment R⁵ and R⁶ represent H and both R⁷ and R⁸ represent —CH₃.

According to a further aspect of the present invention R¹ and R² may together form a hydrocarbon ring group which may be aromatic on non-aromatic. The hydrocarbon ring group comprises an O heteroatom.

The hydrocarbon ring group formed from R¹ and R² in combination may be substituted or unsubstituted. The hydrocarbon ring group may be substituted with one or more of any substituted or unsubstituted alkane, alkene, alkyne or aromatic hydrocarbon group.

Where any of R¹ to R⁸ represent a hydrocarbon ring group, the hydrocarbon ring group may include one or more heteroatoms. The heteroatoms may suitably be N, O or S groups. Where the heteroatom is an N group it may suitably be quaternised.

According to one aspect of the present invention there is provided the compounds of Formula 1 for use in therapy or diagnosis with the proviso that the compound may not be Ochratoxin A.

The term “aryl” refers to any aromatic carbocyclic system containing one or more rings. The hydrocarbon rings may be attached in a pendant (e.g. biphenyl) or fused (e.g. naphthyl) manner. The term aryl further encompasses heteroaryl compounds including aromatic systems containing oxygen, nitrogen or sulphur as one or more ring atoms.

The term “aralkyl” refers to a structure having an alkyl and an aryl component.

In one embodiment of the present invention there is provided compounds for use in therapy, said compounds having the structure defined by Formula 1 above wherein:
R¹ is H;
R² is as defined above;
R³ to R⁸ represent substituted or unsubstituted aliphatic or aromatic hydrocarbon groups, H, OH, OR⁹, OCOR⁹, NH², NR₂⁹, COR⁹, SO₂R⁹, CN, NO₂, halogen, SO₃H, CHO, SH, SR⁹, PO(OR⁹)₂, PO(OR⁹)NR₂⁹, CO₂H, CO₂R⁹, CONR₂⁹ SOR⁹, SO₃R⁹, PC(NR₂⁹)₂, N₃ or SO₂NR₂⁹ wherein R⁹ is as defined above, with the proviso that R⁵ and R⁶, or R⁷ and R⁸ may not simultaneously represent OH, SH or NH₂ and with the further proviso that R⁴ does not represent Cl.

In one embodiment R⁹ represents a C_{1 to 25} alkyl, alkenyl or alkynyl, aryl or araryl group.

In one embodiment of the present invention there is provided compounds for use in therapy, said compounds having the structure defined by Formula 1 above wherein:
R¹, R³, R⁴, R⁵ and R⁶ represent H;
R⁷ and R⁸ represent H or CH₃ where one or both of R⁷ and R⁸ may represent CH₃ or one or both of R⁷ and R⁸ may represent H;
R² is as defined above.

In one embodiment R² represents one of the following groups:

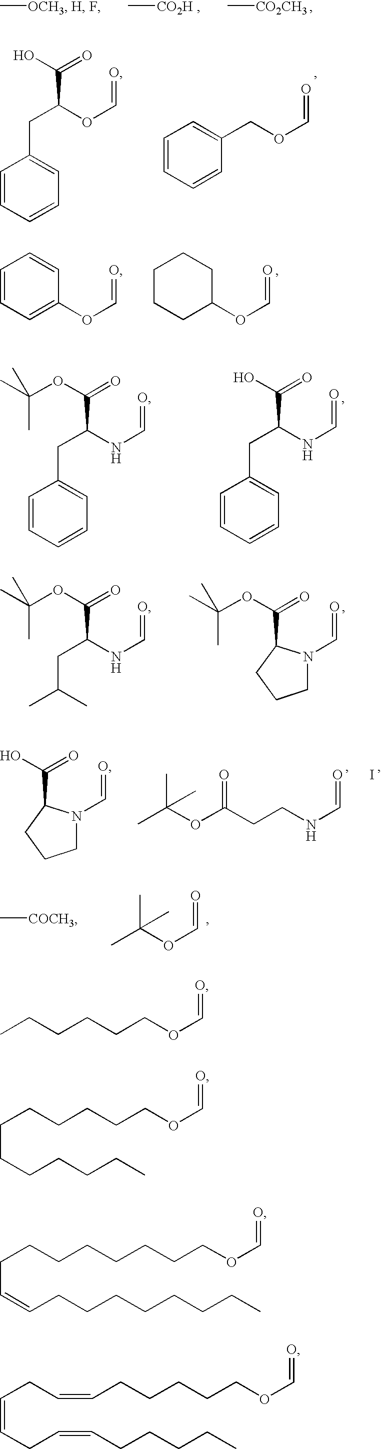

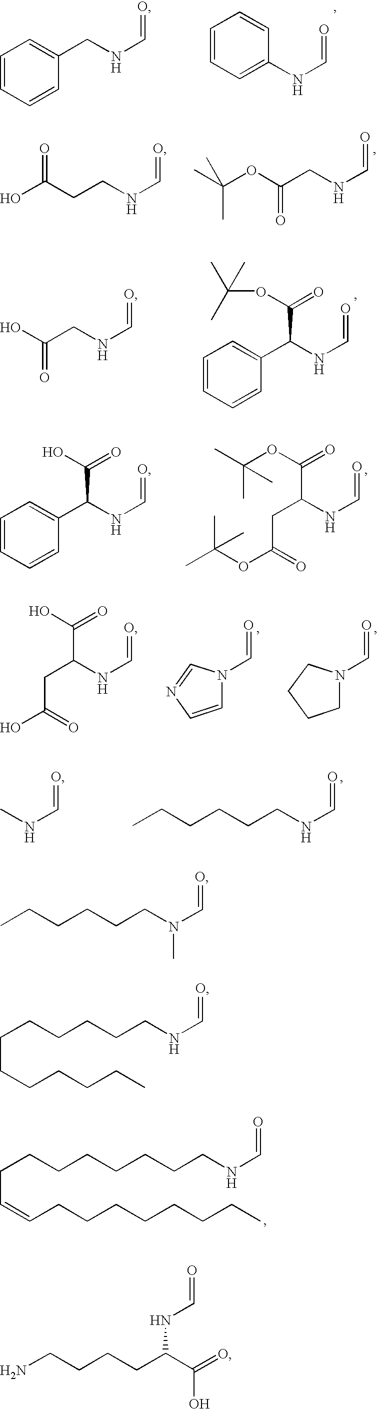

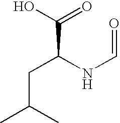

Preferably R² represents one of the following groups:

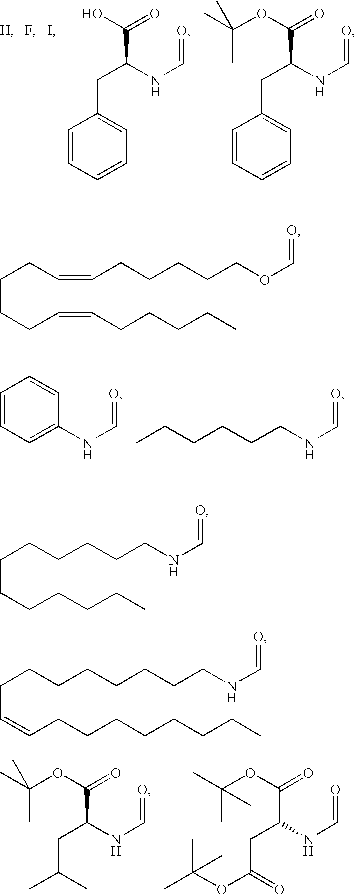

Alternatively R² and R⁴ represent NO₂.

In one embodiment R² and R⁴ represent I.

In one embodiment of the present invention there is provided compounds for use in therapy, said compounds having the structure defined by Formula 1 above wherein:
R¹, R², R³ and R⁴ are as defined above;
R⁵ and R⁶ represent H; and R⁷ and R⁸ represent H or CH₃ where one or both of
R⁷ and R⁸ represents CH₃, or one or both of R⁷ and R⁸ represent H.

In one embodiment R¹ suitably represents one of the following groups:

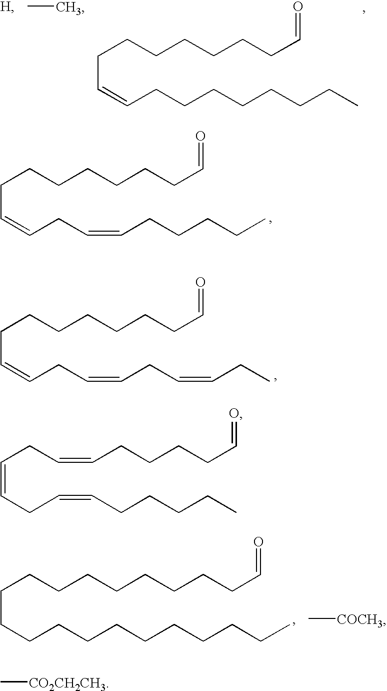

R¹ attaches via the carbonyl group of the structures having a carbonyl group listed above.

In one embodiment R² represents H, —OCH₃, halogen (such as F or I), —COCH₃, or

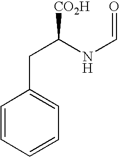

R³ suitably represents H or —OCH₃.

In one embodiment R⁴ represents H, F, Br or I. R⁴ may not represent Cl.

Alternatively R² and R⁴ represent NO₂.

In one embodiment R² and R⁴ represent E.

According to a further aspect of the present invention there is provided a pharmaceutically acceptable salt of the compounds as described above for use in therapy. According to convention the compounds have been described in their closed ring structure. However, it will be understood that the compounds of the present invention may also be in the form of a pharmaceutically acceptable salt of the compounds described above, or may be in the form of an open chain derivative of the structures shown above formed from the hydrolytic opening of the lactone ring of the structures of Formula 1.

Stereoisomers of all the above compounds are possible because of the chiral centres which can occur at positions 3 and 4 of the ring structure in Formula 1.

Different stereoisomers of some of the compounds described above may have different activities (when compared to each other and to the racemate). In particular different stereoisomers of some of the compounds described above may have different anti-proliferative effects against certain cancer cell lines.

Thus according to a further embodiment of the present invention there are provided the R- and S-stereoisomers of the abovementioned compounds for use in therapy. In general terms the present invention envisages that the compounds in question may be used either as racemates and/or as individual and separate stereoisomers.

Suitably the compounds of the present invention (particularly ochratoxin B) are used in therapy in the racemic form.

### Anti-Cancer Activity

According to a further aspect of the present invention there is provided a method of treating neoplasia comprising the steps of administering a compound as described above to a patient.

According to a further aspect of the present invention there is provided the use of a compound as described above in the manufacture of a medicament for the treatment of neoplasia.

In one embodiment of the present invention the neoplasia is cancerous. Suitably the neoplasia may manifest itself as a tumour in the skin, or cancer of the breast, lung, prostate, colon, stomach, upper GI tract, kidney, pancreas, ovary, bladder, head and neck or other recognised solid tumour. Additionally, the malignancy may also manifest itself as a form of leukaemia.

Suitably the compounds described above exhibit anti-proliferative effects against one or more cancer cell lines including SkMe128, MalMe3M, MCF-7, MDA-MB-468, PC3, PNT2, LNCaP, ZR-75-1, HT29, RKO, H157 and H23 cells.

The compounds suitably have an IC 50 value of 1000 μM or less in blocking the proliferation of cancer cell lines. Compounds are said to have preferable activity where IC50 values are 100 μM or less and advantageously so at values lower than 20 μM.

The compounds as described above have been found to be particularly effective as anti-proliferative agents against breast cancer cell lines in vitro. In one embodiment a compound as described above is used in the treatment of breast cancer.

It has been found that the abovementioned compounds act to induce apoptosis in neoplastic cells. Therefore in an alternative embodiment, there is provided a method of inducing apoptosis in a neoplastic cell comprising administering to the cell at least one compound of Formula 1 as previously defined in an amount sufficient to induce apoptosis.

According to a further aspect of the present invention there is provided an assay comprising the steps of:
contacting a sample with at least one compound of Formula 1 as previously defined;
determining if said compound binds to a component of said sample; and
isolating a component which binds said compound.

Such an assay will allow the identification of molecules which interact with compounds of Formula 1, and hence allow determination of their mode of action. This may in turn allow identification of novel targets for anti-proliferative treatment and the development of novel or refined therapeutics.

According to a further aspect of the present invention there is provided an anti-proliferative composition comprising one or more of the compounds described above together with one or more pharmaceutically acceptable excipients. Suitably the pharmaceutically acceptable excipients may include the use of fillers, solvents, surfactants or stabilisers.

The composition may also comprise one or more known cancer drugs since surprisingly it has been found that the compounds of the present invention act synergistically with known cancer drugs in the treatment of neoplasia.

As a further aspect of the present invention, a combination of one or more compounds of the present invention may be combined together with one or more known clinically used cancer drugs to form a composition which exhibits a synergistically high anti-proliferative effect against one or more cancer cell lines.

Suitably the composition has a combination index of less than 0.9; suitably less than 0.7; preferably less than 0.3; advantageously less than 0.1.

A combination index of less than 0.1 indicates very strong synergy; a combination index of 0.1 to 0.3 indicates strong synergism; a combination index of 0.3 to 0.7 indicates synergism; a combination index of 0.7 to 0.85 indicates moderate synergism; a combination index of 0.85 to 0.9 indicates slight synergism and a combination index of 0.9 to 1.1 indicates only very slight synergism, said combination being close to merely additive.

The combination index may be calculated using calcusyn software.

Suitably the composition exhibits synergistically high anti-proliferative effects against one or more of the following cancer cell lines SkMe128, MalMe3M, MCF-7, MDA-MB-468, PC3, PNT2, LNCaP, ZR-75-1, HT29, RKO, H157 and H23 cells.

In one embodiment the composition exhibits a synergistically high anti-proliferative effect against the MDA-MB-468 cell line.

In one embodiment the cytotoxicity of the known cancer drug(s) is increased against skin tumour cells or cancer cells of the breast, lung, prostate, colon, stomach, upper GI tract, kidney, pancreas, ovary, bladder, head or neck.

In one embodiment the cytotoxicity of the known drugs (such as vinorelbine, irinotecan, cisplatin, etoposide, docetaxel and doxorubicin) against neoplasms is synergistically increased through combination with one or more compounds of the present invention.

Vinorelbine has the empirical formula C₄₄H₅₂N₄O₈. Irinotecan has the empirical formula C₃₃H₃₈N₄O₆.HCl.3H₂O and the chemical name (S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate, monohydrochloride, trihydrate. Cisplatin has the chemical name cis-diaminedichloro-platinum(S)-4,11-diethyl-3,4,12,14. Etopside has the chemical name 4′-Demethylepipodophyllotoxin 9-[4,6-0-(R)-ethylidene-β-D-glucopyranoside], 4′-(dihydrogen phosphate). Docetaxel has the empirical formula C₄₃H₅₃NO₁₄ and doxorubicin has the chemical name 10-(4-amino-5-hydroxy-6-methyl-oxan-2-yl)oxy-6,8,11-trihydroxy-8-(2-hydroxyacetyl)-1-methoxy-9,10-dihydro-7H-tetracene-5,12-dione and the empirical formula C₂₇H₂₉NO₁₁.

The synergistic composition of the present invention preferably comprises one or more of the compounds of the present invention as described above together with one or more microtubule-disrupting agent such as vinorelbine and docetaxel, a topoisomerase I-targeted agent such as irinotecan etoposide and doxorubicin and a DNA damaging agent such as cisplatin.

In one aspect of the present invention the synergistic composition comprises one of the compounds of the present invention as described above together with one or more of vinorelbine, irinotecan, cisplatin, etoposide, docetaxel or doxorubicin.

Preferably the synergistic composition comprises one of the compounds of the present invention as described above together with vinorelbine, irinotecan, cisplatin, etoposide or doxorubicin.

Suitably the composition includes one or more compounds according to Formula 1 wherein:
R¹ is H;
R² is as defined above;
R³ to R⁸ represent substituted or unsubstituted aliphatic or aromatic hydrocarbon groups, H, OH, OR⁹, OCOR⁹, NH², NR₂⁹, COR⁹, SO₂R⁹, SOR⁹, CN, NO₂, halogen, SO₃H, CHO, SH, SR⁹, PO(OR⁹)₂, PO(OR⁹)NR₂⁹, CO₂H, CO₂R⁹, CONR₂⁹ SO₃R⁹, PO(NR₂⁹)₂, N₃ or SO₂NR₂⁹ wherein R⁹ is as defined above, with the proviso that R⁵ and R⁶, or R⁷ and R⁸ may not simultaneously represent OH, SH or NH₂, and with the further proviso that R⁴ does not represent Cl.

Alternatively the composition includes one or more compounds according to Formula 1 wherein:
R¹, R³, R⁴, R⁵ and R⁶ represent H;
R⁷ and R⁸ represent H or CH₃ where one or both of R⁷ and R⁸ may represent CH₃ or one or both of R⁷ and R⁸ represent H;
R² is as defined above.

In one embodiment R² represents one of the following groups:

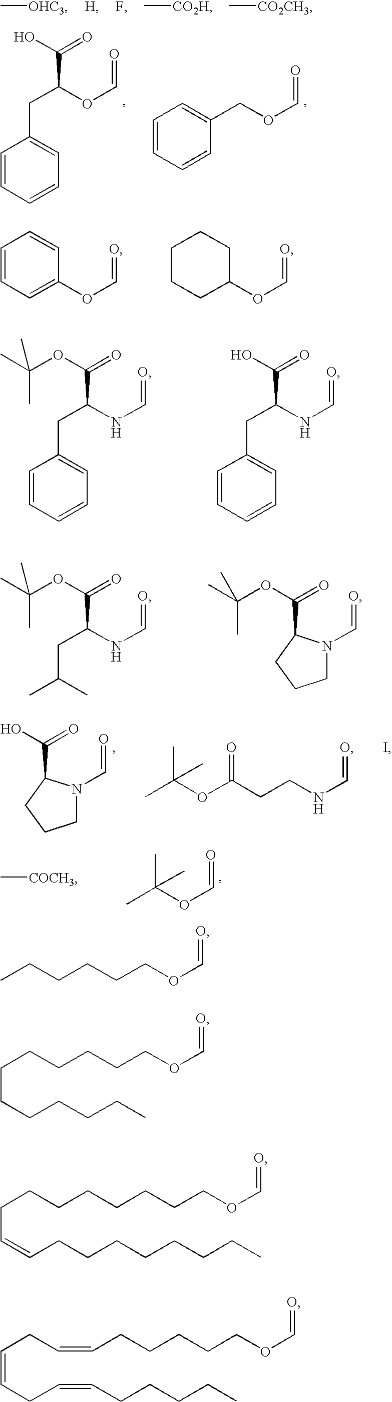

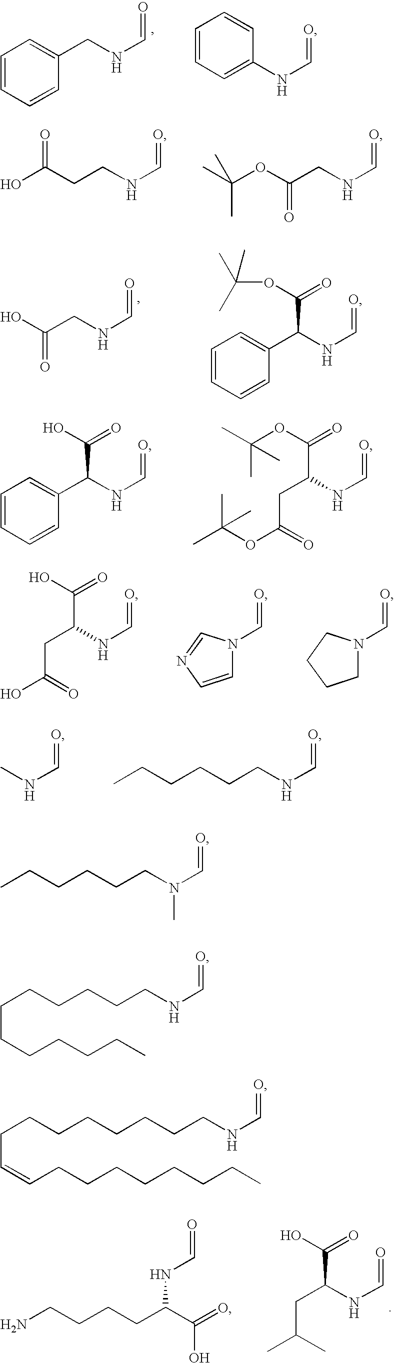

R² preferably represents one of the following groups:

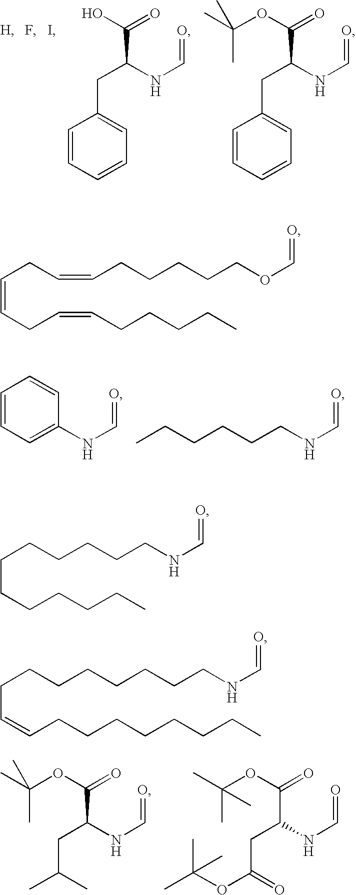

Alternatively R² and R⁴ represent NO₂

In one embodiment R² and R⁴ represent I.

In a further aspect of the present invention the composition comprises one or more compounds according to Formula 1 wherein:
R⁵ and R⁶ represent H;
R⁷ and R⁸ represent H or CH₃ where one or both of R⁷ and R⁸ may represent CH₃, or one or both of R⁷ and R⁸ may represent H.
R¹ represents one of the following groups:
H, CH₃

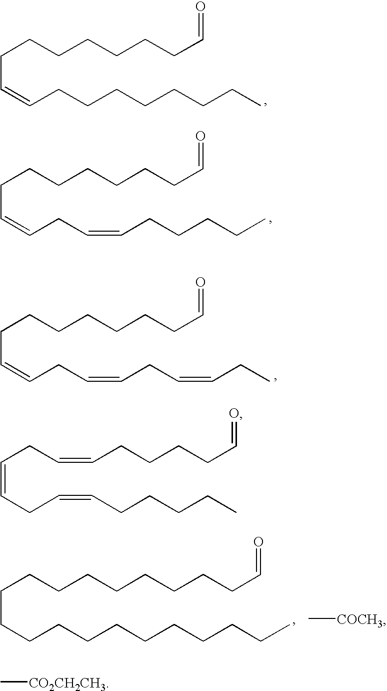

R¹ attaches via the carbonyl group of the structures having a carbonyl group listed above.

In one embodiment R² represents H, halogen (such as F or I), —COCH₃, or

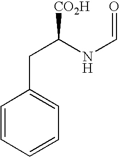

R³ suitably represents H or —OCH₃.

In one embodiment R⁴ represents H, F, Br or I.

R⁴ may not represent Cl.

Alternatively R² and R⁴ represent NO₂.

In one embodiment R² and R⁴ represent I.

In one embodiment the composition comprises one or more of ALM-43, ALM-49, ALM-54, ALM-55, ALM-65 and ALM-74 (as defined in Table 1.1 below).

Suitably the composition includes one or more compounds of the present invention at a concentration of up to 5 μg/ml; suitably 0.1 to 0.5 μg/ml.

Suitably the composition comprises ALM-43 and vinorelbine, etoposide, cisplatin, irinotecan, docetaxel or doxorubicin.

In one embodiment the composition comprises ALM-43 at a concentration of 0.1 to 0.5 μg/ml; suitably 0.1 μg/ml. Suitably the composition comprises 0.1 nM to 100 nM etoposide.

In one embodiment the composition comprises 1 nM to 100 nM cisplatin.

In one embodiment the composition comprises 0.1 nM to 10 nM irinotecan.

In one embodiment the composition comprises 10 nM to 10 μM doxorubicin.

Suitably the composition comprises ALM-49 and etoposide, cisplatin, doxorubicin, docetaxel, vinorelbine or irinotecan.

Suitably the composition comprises 0.1 to 5 μg/ml ALM-49; more suitably 0.1 μg/ml ALM-49.

In one embodiment the composition comprises 0.1 nM to 1 nM etoposide.

In one embodiment the composition comprises 1 nM to 100 nM cisplatin.

Suitably the composition comprises 10 nM to 10 μM doxorubicin.

Suitably the composition comprises ALM-55 and etoposide, cisplatin, doxorubicin, vinorelbine, docetaxel, or irinotecan.

Suitably the composition comprises 0.1 to 1.0 μg/ml ALM-55; more suitably 0.1 to 0.5 μg/ml ALM-55, appropriately 0.1 μg/ml ALM-55.

In one embodiment the composition comprises 0.1 nM to 10 Nm etoposide.

In one embodiment the composition comprises 1 nM to 100 nM cisplatin.

Suitably the composition comprises 0.01 nM to 10 μM doxorubicin; appropriately 1 nM to 1 μM.

Suitably the composition comprises ALM-65 and etoposide, cisplatin, doxorubicin, vinorelbine, docetaxel, or irinotecan.

Suitably the composition comprises 0.1 to 5.0 μM/ml ALM-65. In one embodiment the composition comprises 0.1 nM to 10 nM etoposide.

Suitably the composition comprises 1 nM to 1 μm cisplatin.

Suitably the composition comprises 0.1 nM to 1 μM irinotecan.

In one embodiment the composition comprises 0.01 nM to 10 μM doxorubicin; suitably 1 mM to 10 μM doxorubicin; more suitably 10 nm to 1 μM doxorubicin.

According to a further aspect of the present invention the composition comprises ALM-74 and etoposide, cisplatin, doxorubicin, vinorelbine, docetaxel, or irinotecan.

Suitably the composition comprises 0.1 to 5 μg/ml ALM-74; more suitably 1 to 5 μg/ml ALM-74.

In one embodiment the composition comprises 0.1 nM to 100 μM etoposide.

Suitably the composition comprises 1 nM to 100 μM cisplatin. Suitably the composition comprises 0.1 nM to 100 μM irinotecan.

In one embodiment the composition comprises 0.01 nm to 10 μM doxorubicin.

The composition is suitably in the form of a pharmaceutically acceptable formulation, such as livid, semi-solid and solid oral preparations, chewing gum preparations, ear preparations, eye preparations, foam preparations, granule preparations, intramammary preparations, intraruminal preparations, liquid, semi-solid and solid cutaneous and transdermal preparations, nasal preparations, parenteral preparations, premix preparations for feeding stuffs, preparations for inhalation, preparations for irrigation, pressurised preparations, rectal preparations, subcutaneous preparations, tampon preparations, vaginal preparations, intravaginal preparations, implantable preparations, oromucosal preparations, preparations for dental use, tracheopulmonary preparations, preparations for dialysis, endocervical preparations, intrauterine preparations, preparations for intravesical and urethral use.

Unless otherwise states the term “preparation” should be taken to mean any pharmaceutical dosage form, delivery system or device. Each of the above principal examples are to be taken to include all sub-sections within that example.

In one embodiment the present invention provides the composition as described above for use in therapy.

According to a further aspect of the present invention there is provided a method of treatment of neoplasia comprising the steps of administering the composition as described above to a patient.

The components of the composition may be administered separately or simultaneously, suitably in the same preparation.

Advantageously the components of the composition are administered simultaneously in the same preparation.

The composition may be administered either as a complete therapy or in combination with other cytotoxic- or biologically-targeted therapeutic strategies known in the treatment of neoplasia.

According to a further aspect of the present invention there is provided the use of the composition as described above in the manufacture of a medicament for the treatment of neoplasia.

According to a further aspect of the present invention there is provided the compounds of Formula 1 as described above with the proviso that the compounds may not be ALM-1, ALM-7 to ALM-15, ALM-24 or ALM-32 to ALM-34 as defined in Table 1.1 below.

According to a further aspect to the present invention there is provided compounds according to Formula 1 above wherein:
R¹, R³, R⁴, R⁵ and R⁶ represent H;
R⁷ and R⁸ represent CH₃ or H where one or both of R⁷ and R⁸ may represent CH₃, or one or both or R⁷ and R⁸ may represent H;
R² represents one of the following groups:

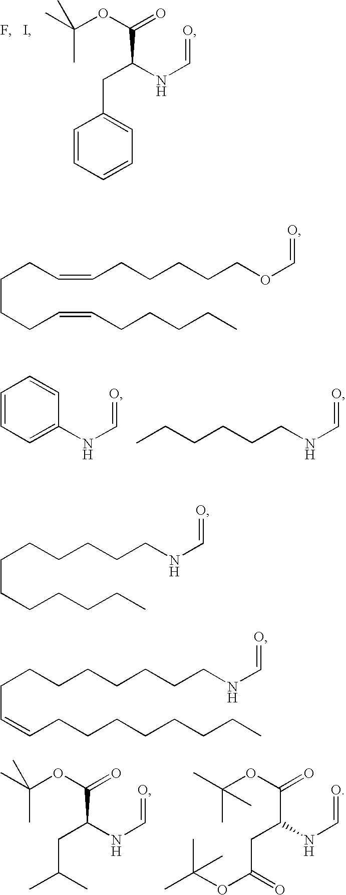

According to a further aspect of the present invention there is provided a pharmaceutically acceptable salt of the compounds described above.

The present invention will now be described by way of example only with reference to the accompanying Figures in which:

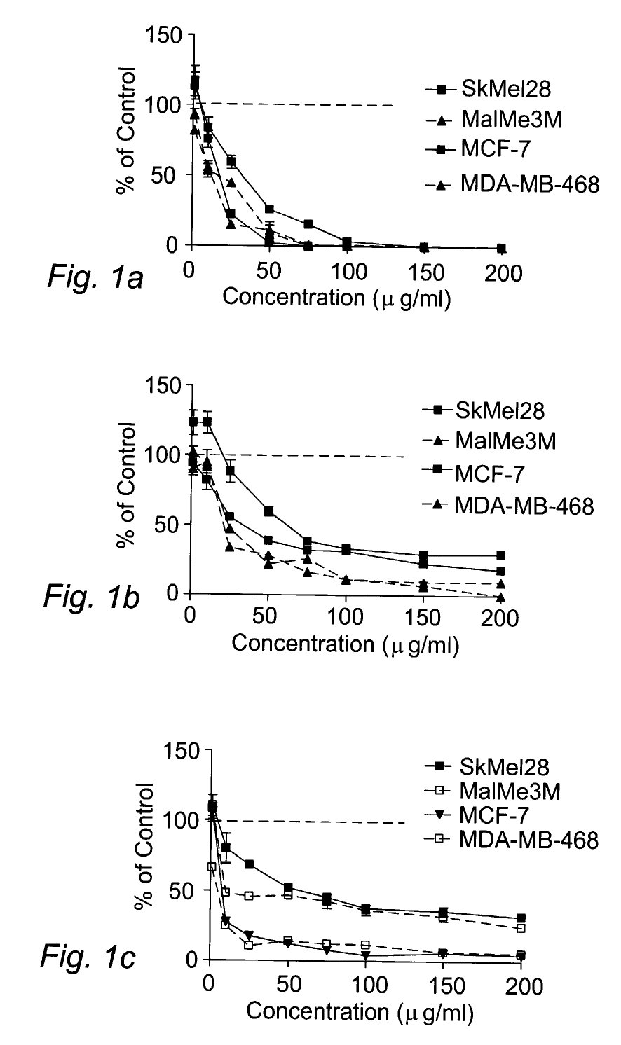

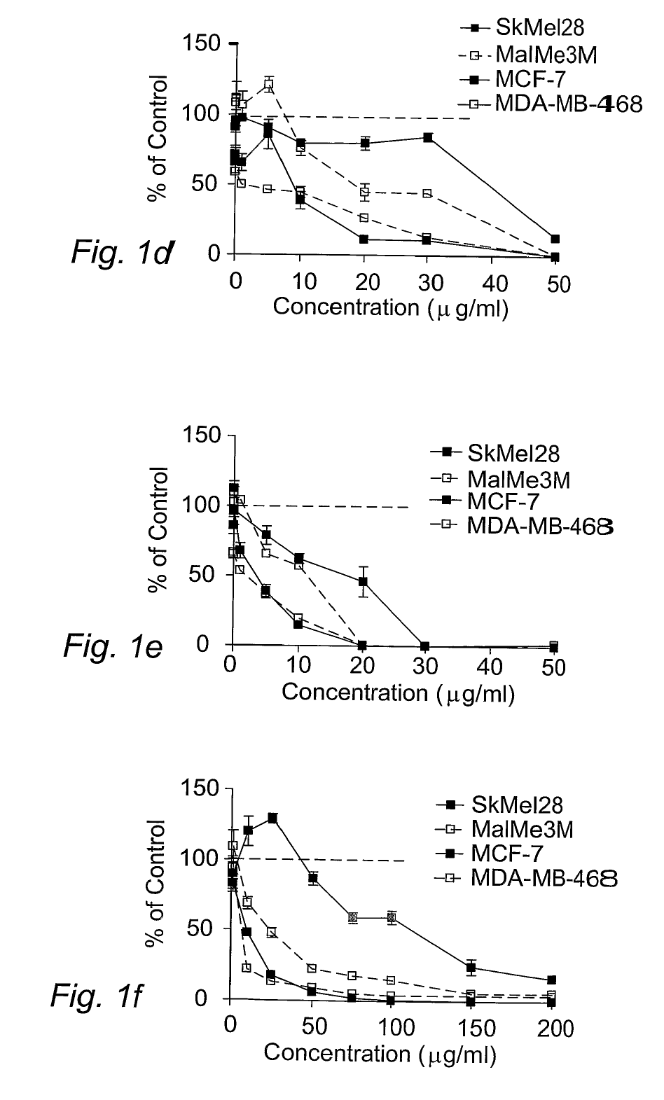

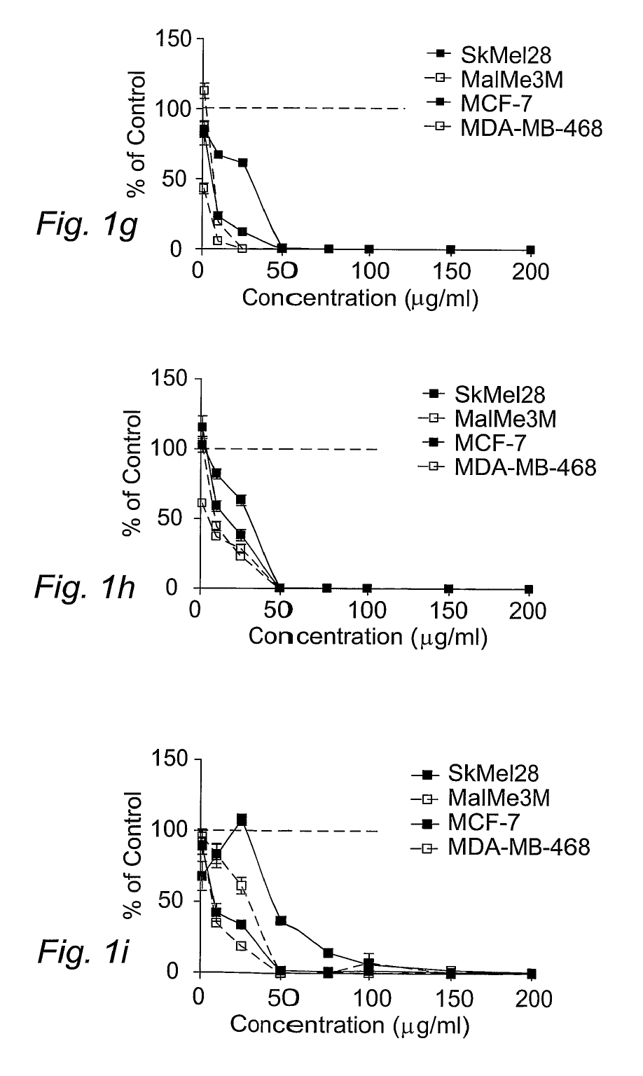

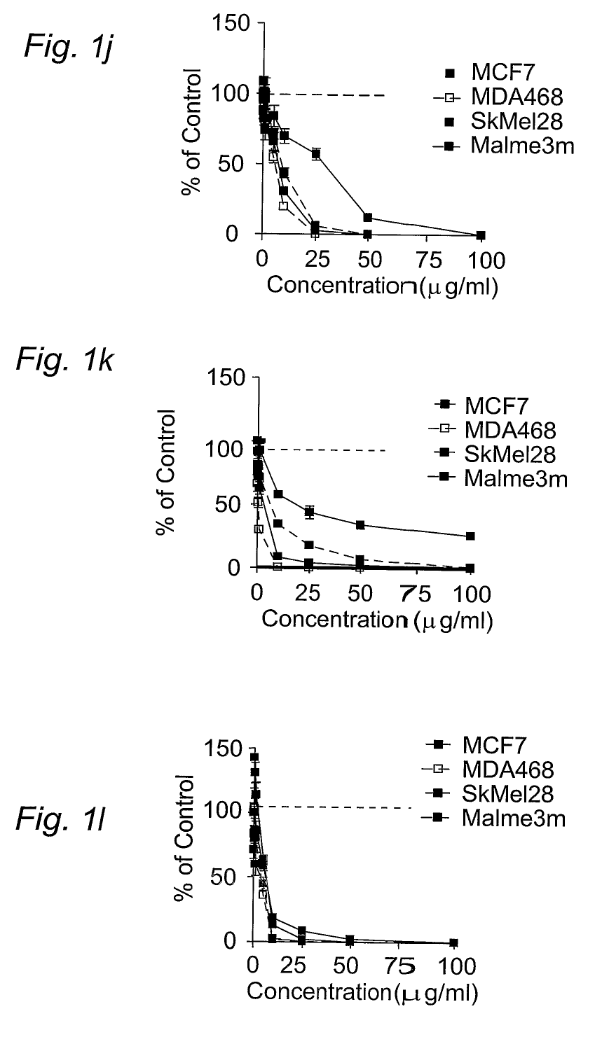

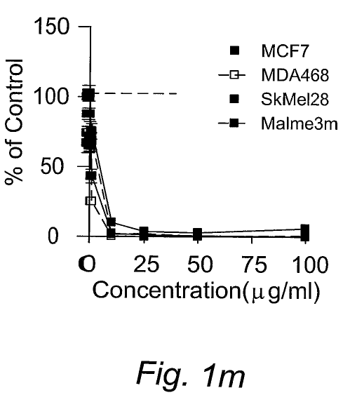

FIGS. 1a to 1m show the anti-proliferative activity of compounds ALM-22, ALM-25, ALM-45, ALM-49, ALM-51, ALM-52, ALM-53, ALM-55, ALM-65, ALM-69, ALM-70, ALM-73 and ALM-74 respectively on breast cancer and melanoma cell lines as measured in MTT assays;

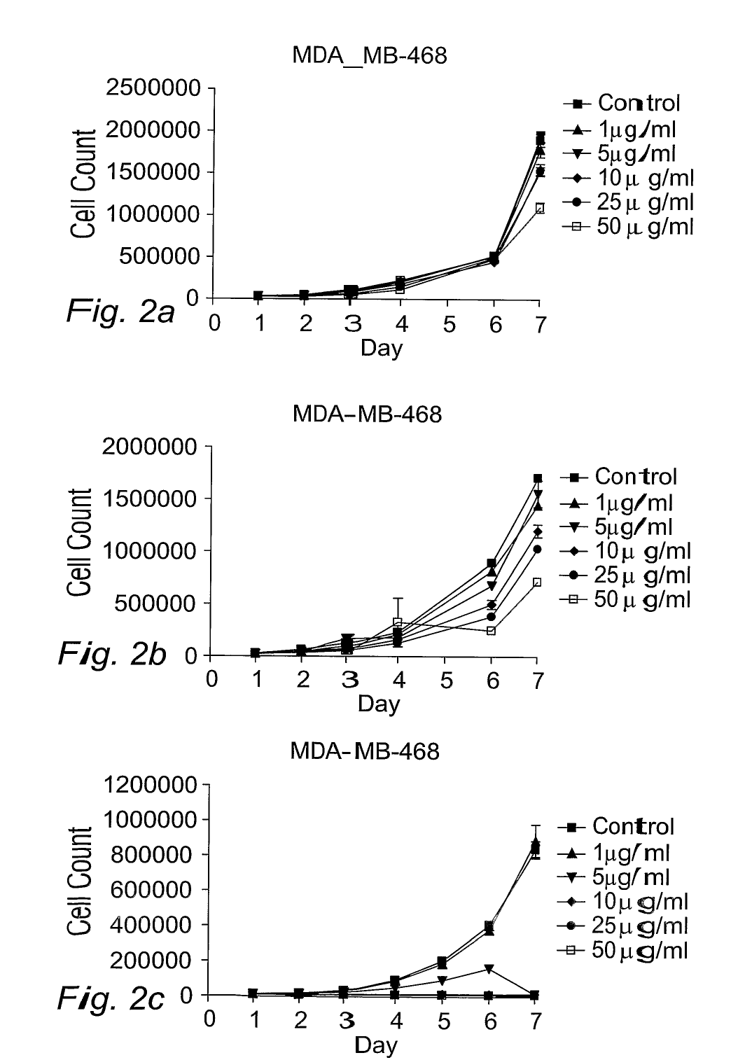

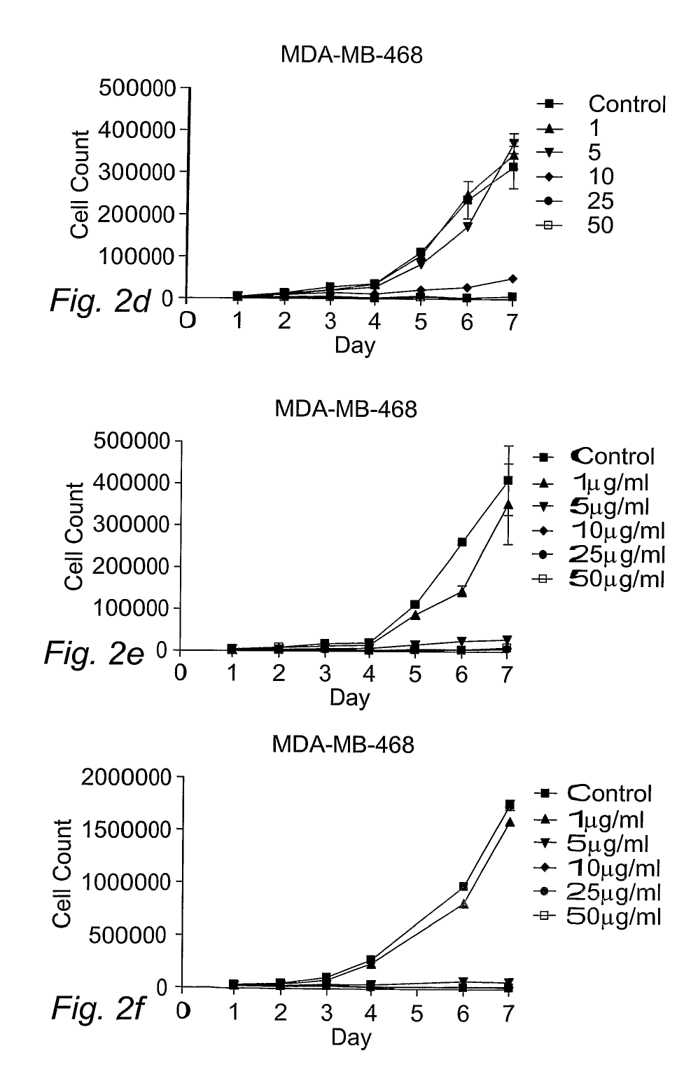

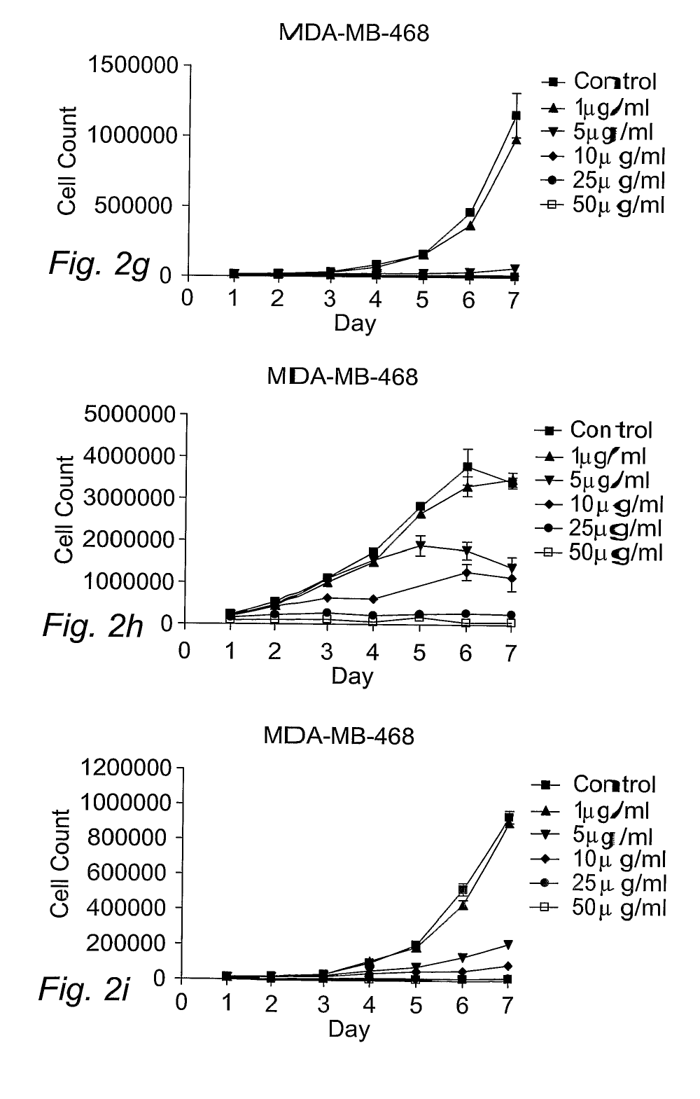

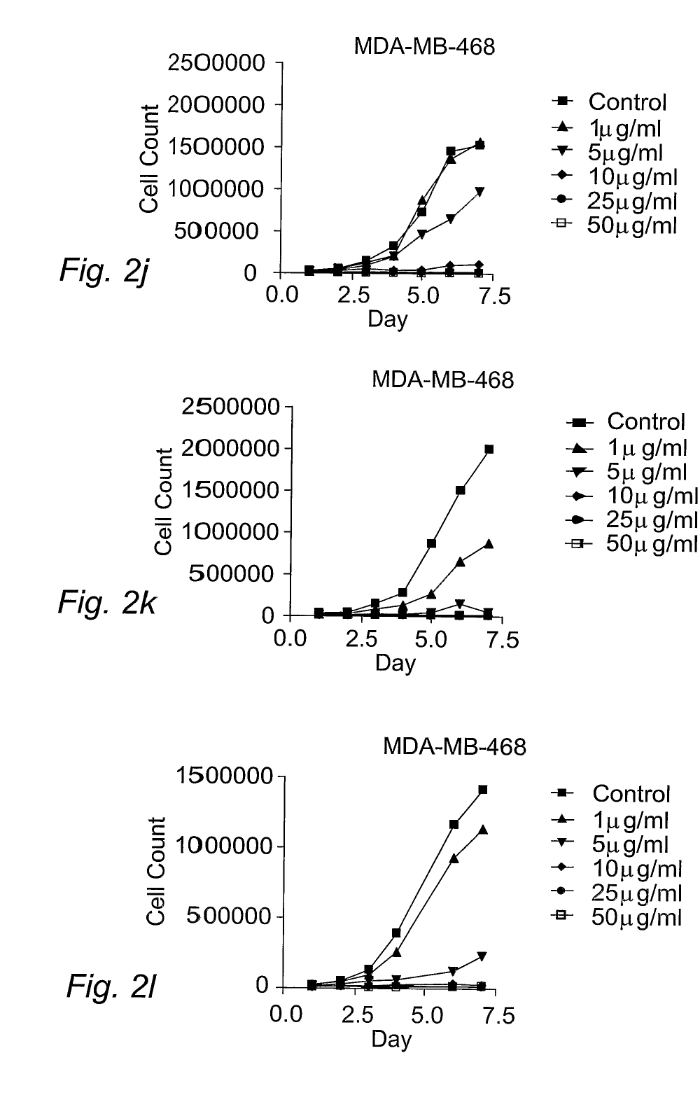

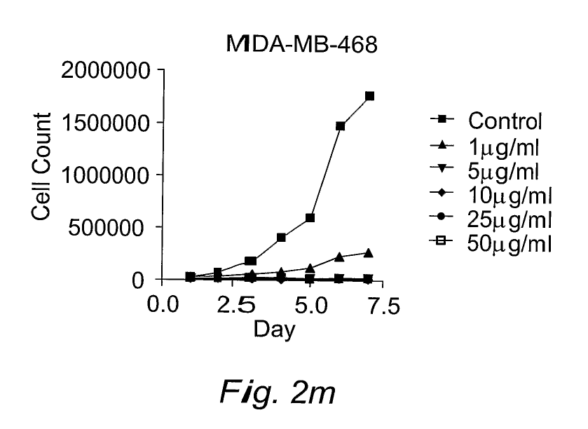

FIGS. 2a to 2m show the anti-proliferative activity on cell line MDA-MB-468 of compounds ALM-22, ALM-25, ALM-45, ALM-49, ALM-51, ALM-52, ALM-53, ALM-55, ALM-65, ALM-67, ALM-70, ALM-73 and ALM-74 respectively over time at varying concentrations as measured in cell count assays;

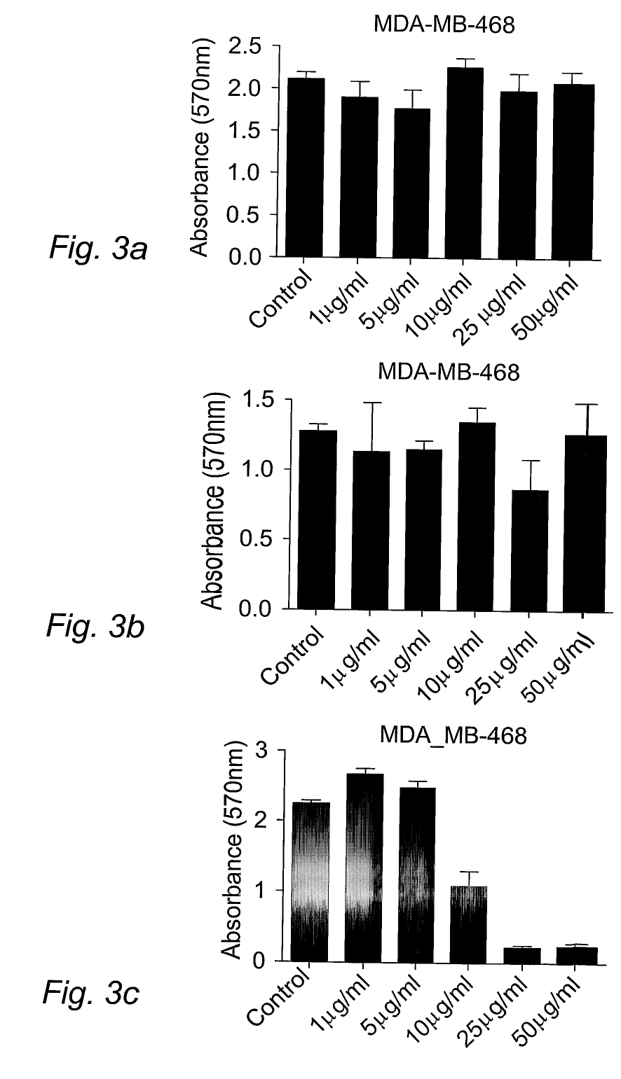

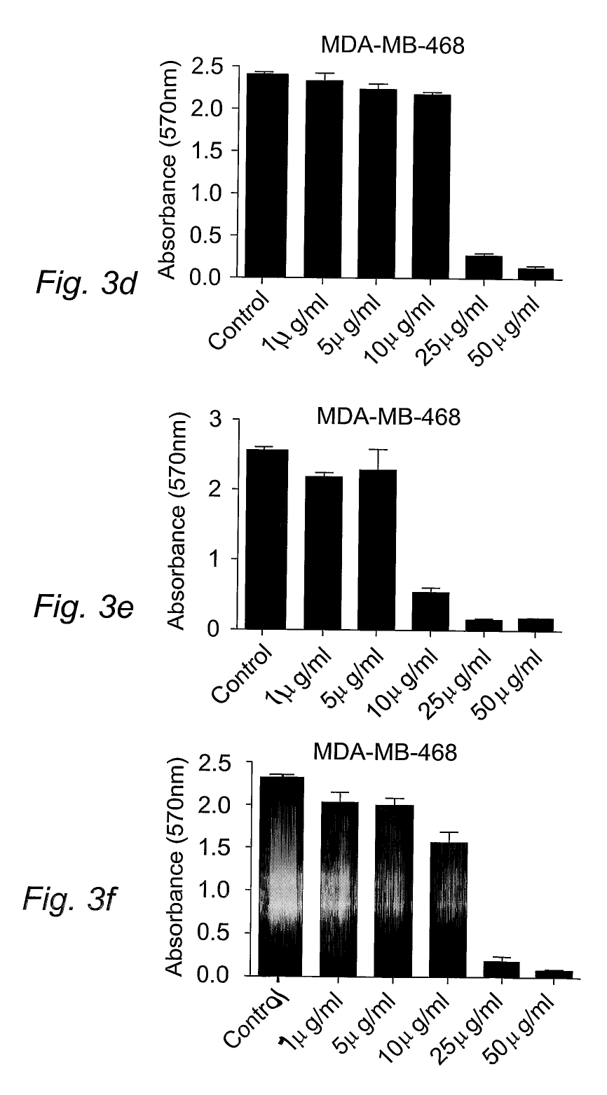

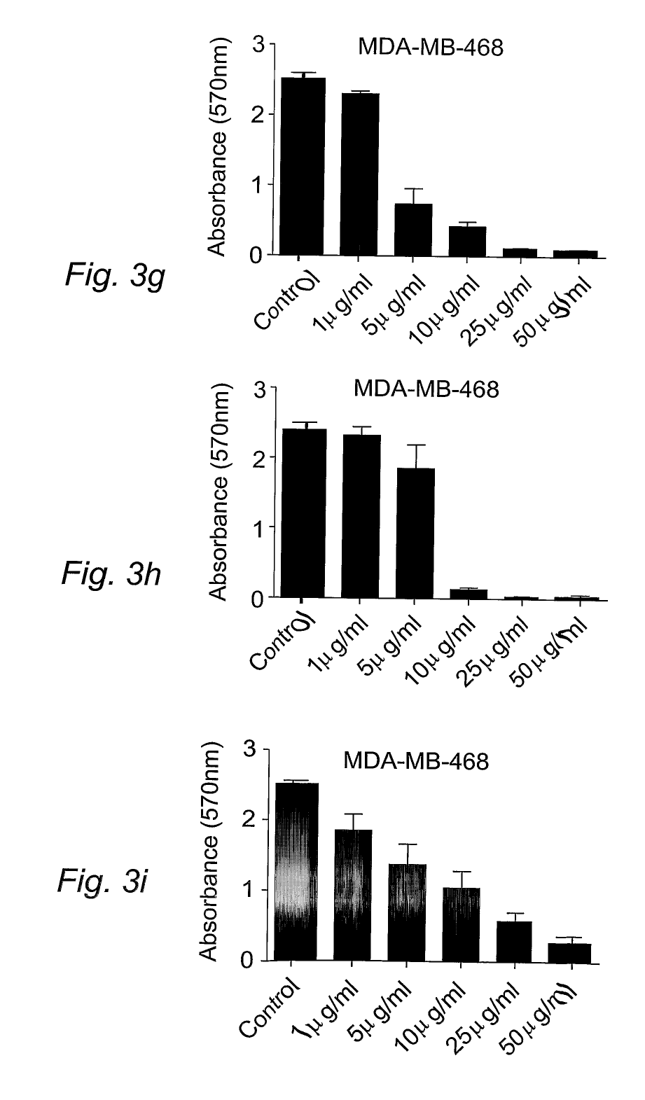

FIGS. 3a to 3i show the anti-proliferative activity on cell line MDA-MB-468 of compounds ALM-22, ALM-25, ALM-45, ALM-49, ALM-51, ALM-52, ALM-53, ALM-55 and ALM-65 respectively at varying concentrations as measured in colony count assays;

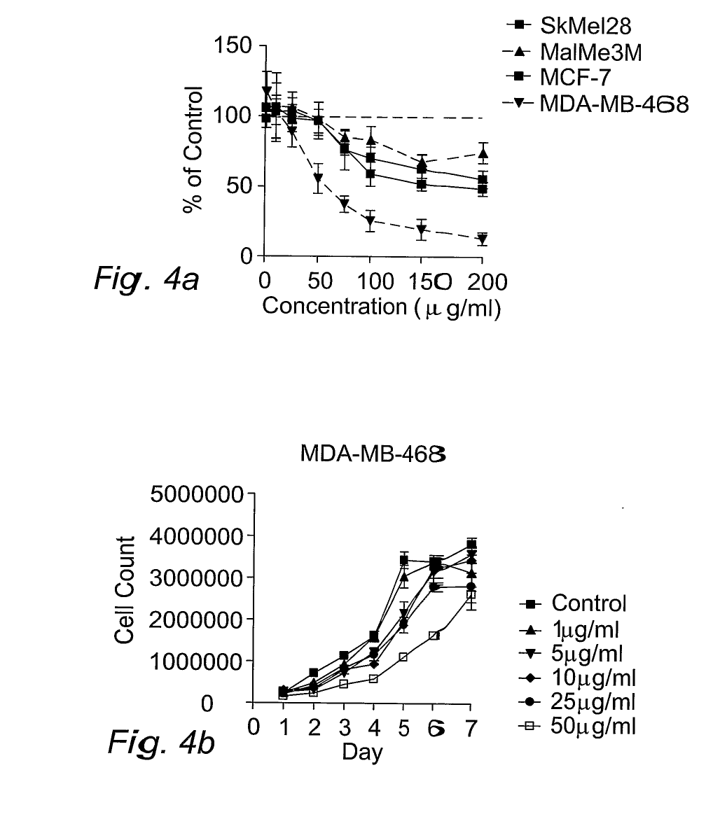

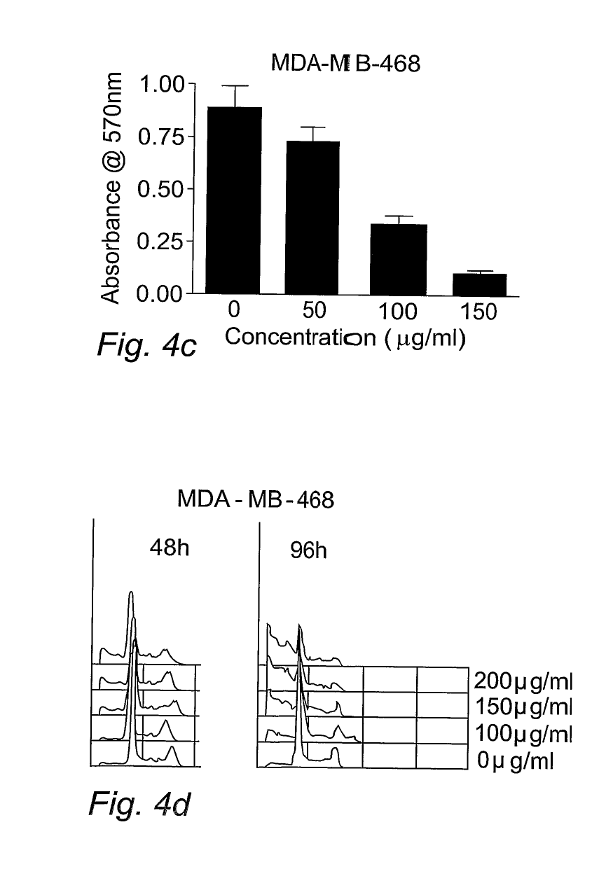

FIG. 4a shows the anti-proliferative activity of compound ALM-9 on breast cancer and melanoma cell lines as measured in an MTT assay;

FIG. 4b shows the anti-proliferative activity of compound ALM-9 on cell line MDA-MB-468 over time at varying concentrations as measured in cell count assays;

FIG. 4c shows the concentration-dependent anti-proliferative activity of compound ALM-9 on cell line MDA-MB-468 over time as measured in colony count assays;

FIG. 4d shows a characterisation of the concentration-dependent anti-proliferative activity of ALM-9 upon the cell profile of cell line MDA-MB-463 cells after exposure to the compound for 48 or 96 hours;

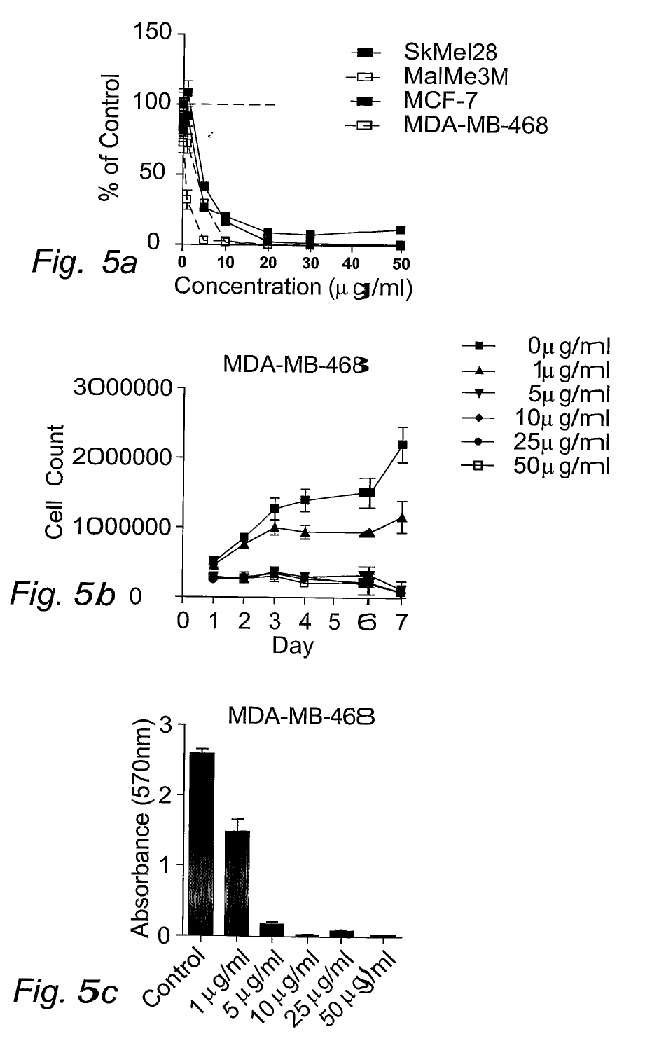

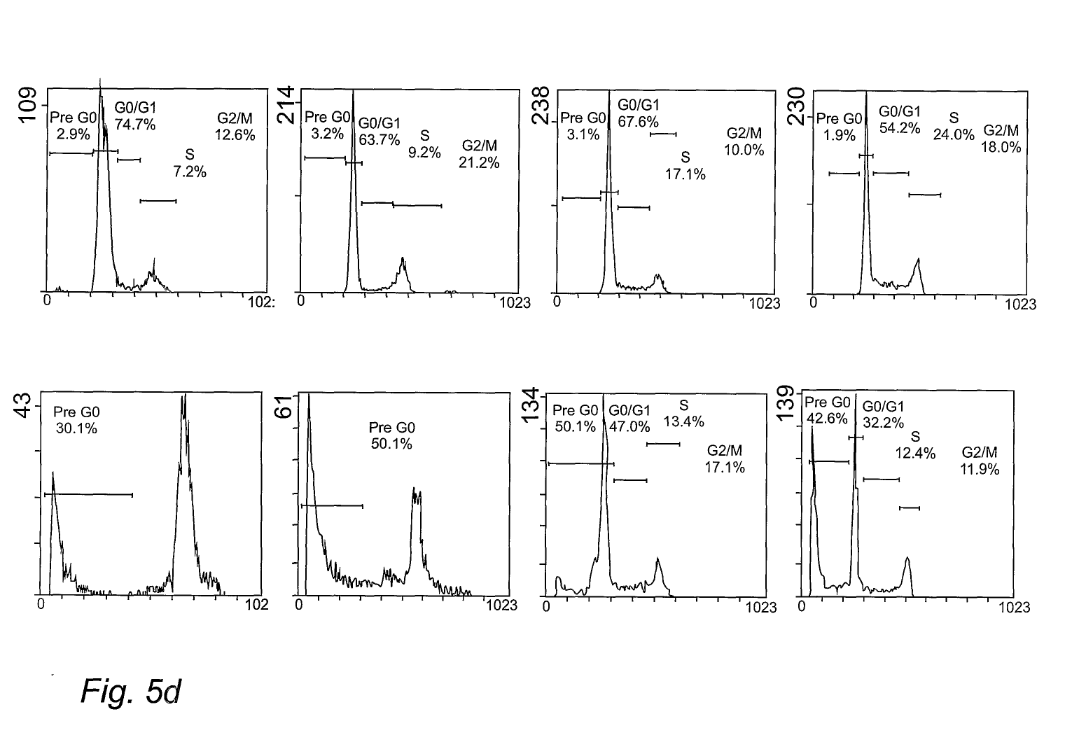

FIG. 5a shows the anti-proliferative activity of compound ALM-54 on breast cancer and melanoma cell lines as measured in MTT assays;

FIG. 5b shows the concentration-dependent anti-proliferative activity of compound A-LM-54 on cell line MDA-MB-468 as measured in cell count assays;

FIG. 5c shows the concentration-dependent anti-proliferative activity of compound ALM-54 on cell line MDA-MB-468 as measured in colony count assays;

FIG. 5d shows flow cytometry profiles demonstrating the effect of ALM-54 at a concentration of 50 μg/ml in altering the cell cycle profile of various breast cancer and melanoma cell lines upon contact with the cell line for 96 hours (cell cycle profiles are shown for each cell line in the absence and in the presence of compound ALM-54);

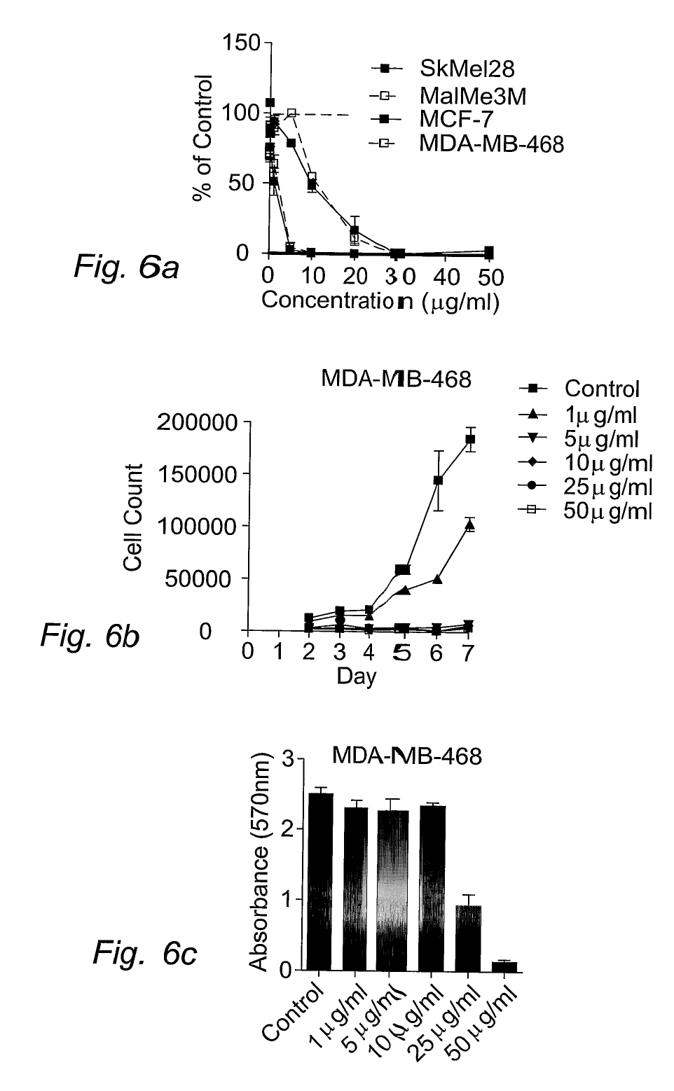

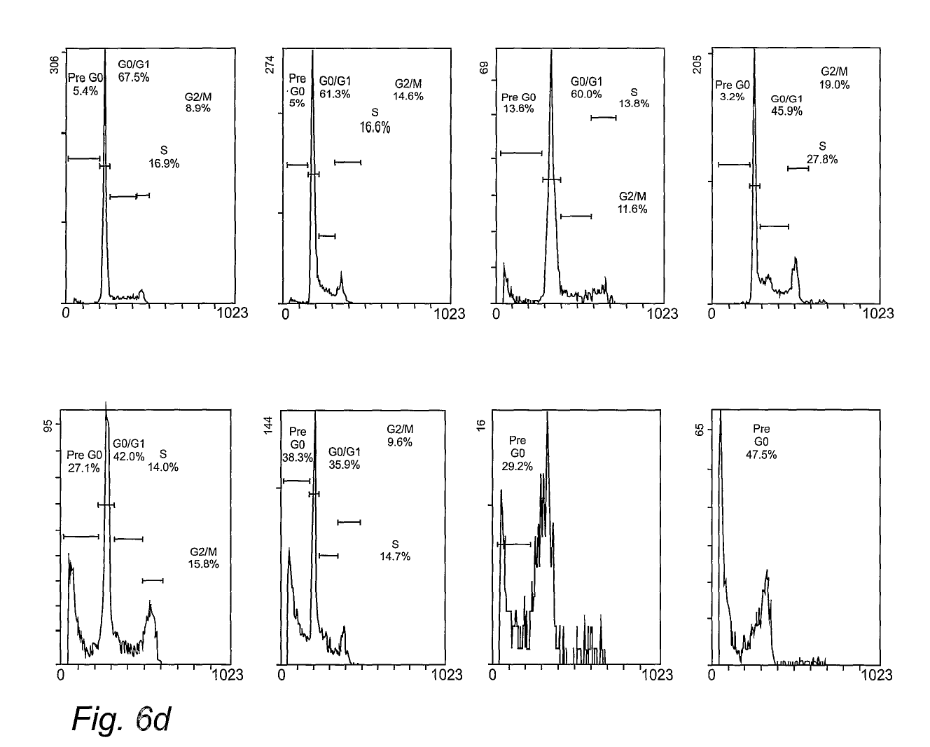

FIG. 6a shows the anti-proliferative activity of compound ALM-43 on breast cancer and melanoma cell lines as measured in MTT assays;

FIG. 6b shows the concentration-dependent anti-proliferative activity of compound ALM-43 on cell line MDA-MB-468 as measured in cell count assays;

FIG. 6c shows the concentration-dependent anti-proliferative activity of compound ALM-43 on cell line MDA-MB-468 as measured in colony count assays;

FIG. 6d shows flow cytometry profiles demonstrating the effect of ALM-43 at a concentration of 50 μg/ml in altering the cell cycle profile of various breast cancer and melanoma cell lines upon contact with the cell line for 96 hours (cell cycle profiles are shown for each cell line in the absence and in the presence of compound ALM-43);

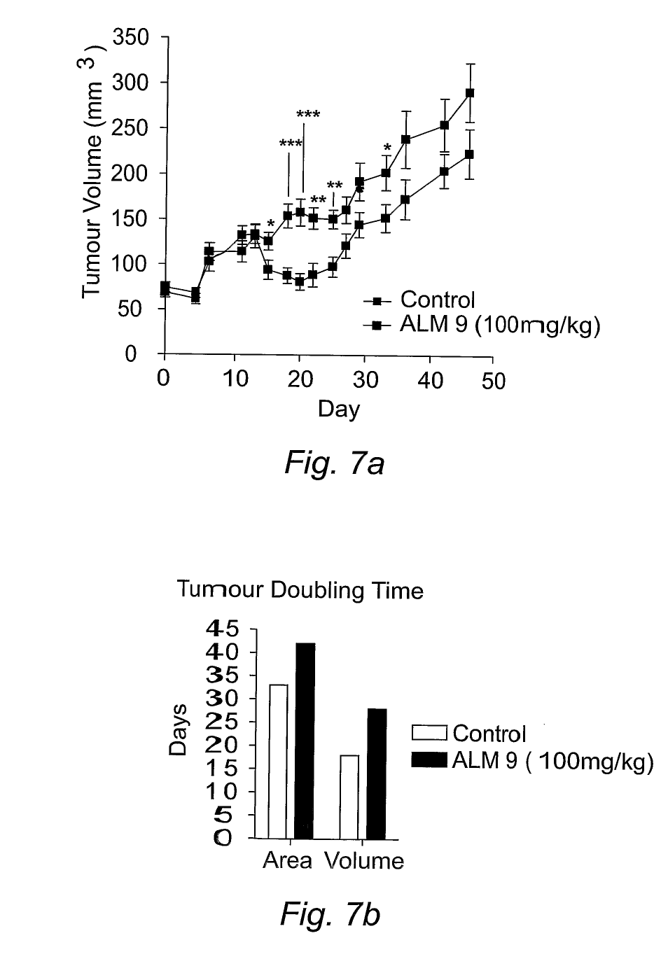

FIG. 7a shows the effect of the administration of compound ALM-9 at a concentration of 100 mg/kg upon the volume of breast cancer xenografts growing in the mammary fat pad of athymic nude mice compared to a control;

FIG. 7b shows a graphical representation of the effect of the administration of compound ALM-9 upon the doubling time of breast cancer xenografts measured as a function of tumour area and tumour volume compared to a control (it was noted that administration of ALM-9 at a concentration of 100 mg/kg had no obvious adverse toxicology determined by behavioural analysis of the mice during the study and by post-mortem histopathological analysis of various organs by a trained pathologist at the conclusion of the study);

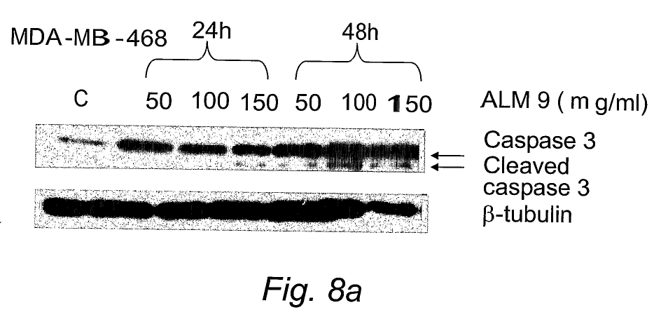

FIG. 8a shows an immunoblot demonstrating that compound ALM-9 promotes the cleavage and thus the activation of caspase 3 at concentrations similar to those required to kill MDA-MB-468 breast cancer cells;

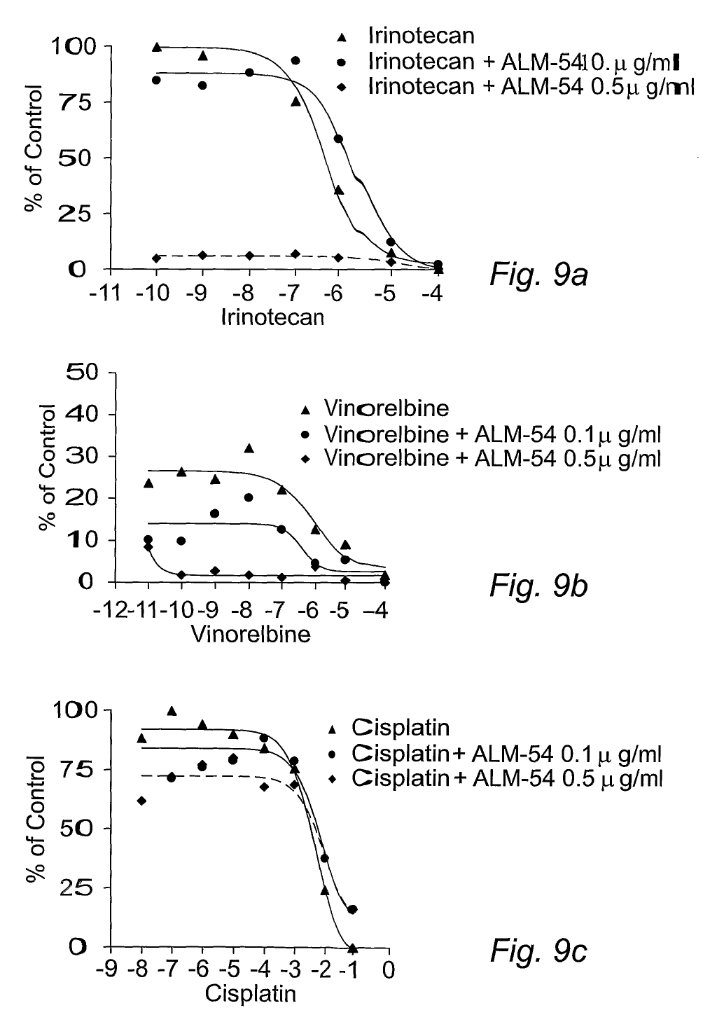

FIGS. 9a, 9b and 9c demonstrate the synergy between compound ALM-54 at a concentration of 0.11 g and a concentration of 0.5 μg and irinotecan, vinorelbine and cisplatin respectively as measured using an MTT assay.

Preferred compounds of the present invention are listed in Table 1.1 below. The numbering of the compounds as shown in Table 1.1 will be used throughout the specification.

TABLE 1.1
Structural Characterisation of Compounds
Class A^{1,2}:

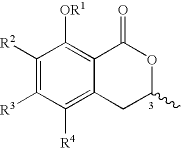

| Compound | | | | |
|---|---|---|---|---|
| No. | R¹ = | R² = | R³ = | R⁴ = |
| ALM-1 | H | —OCH₃ | —OCH₃ | H |
| ALM-2 | 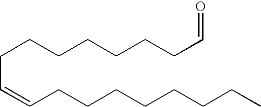 | —OCH₃ | —OCH₃ | H |
| ALM-3 | 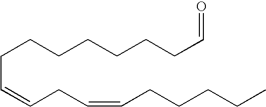 | —OCH₃ | —OCH₃ | H |
| ALM-4 | 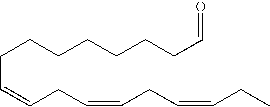 | —OCH₃ | —OCH₃ | H |
| ALM-5 | 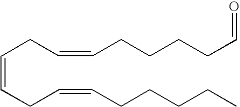 | —OCH₃ | —OCH₃ | H |
| ALM-6 | 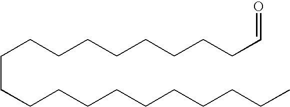 | —OCH₃ | —OCH₃ | H |
| ALM-7 | —CH₃ | H | —OCH₃ | H |
| ALM-8 | H | H | —OCH₃ | H |
| ALM-9 | H | H | H | H |
| ALM-10³ | H | H | H | H |
| ALM-11⁴ | H | H | H | H |
| ALM-12 | —CH₃ | H | H | H |
| ALM-13³ | —CH₃ | H | H | H |
| ALM-14⁴ | —CH₃ | H | H | H |
| ALM-15 | —COCH₃ | H | H | H |
| ALM-16 | —CO₂CH₂CH₃ | H | H | H |
| ALM-17 | 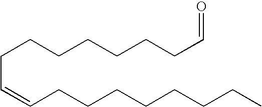 | H | H | H |
| ALM-18 | 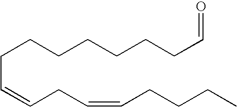 | H | H | H |
| ALM-19 | 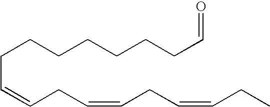 | H | H | H |
| ALM-20 | 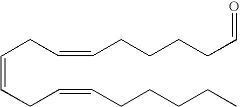 | H | H | H |
| ALM-21 | 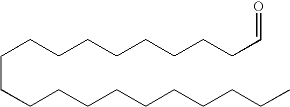 | H | H | H |
| ALM-22 | H | F | H | H |
| ALM-23 | H | H | H | F |
| ALM-24 | H | H | H | Br |
| ALM-25 | H | I | H | H |
| ALM-26 | H | H | H | I |
| ALM-27 | H | I | H | I |
| ALM-28 | —CH₃ | H | H | I |
| ALM-29 | H | —COCH₃ | H | H |
| ALM-30 | H | NO₂ | H | NO₂ |

Class B^{5,6}:

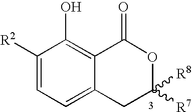

| Compound | | | |
|---|---|---|---|
| No. | R² = | R⁷ = | R⁸ = |
| ALM-32 | 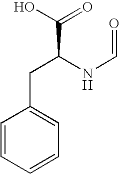 | CH₃ | H |
| ALM-33 | —CO₂H | CH₃ | H |
| ALM-34 | —CO₂CH₃ | CH₃ | H |
| ALM-35 | 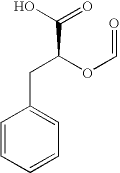 | CH₃ | H |
| ALM-36 | 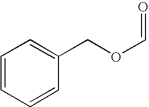 | CH₃ | H |
| ALM-37 | 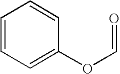 | CH₃ | H |
| ALM-38 | 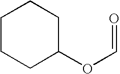 | CH₃ | H |
| ALM-39 | 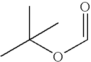 | CH₃ | H |
| ALM-40 | 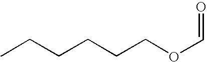 | CH₃ | H |
| ALM-41 | 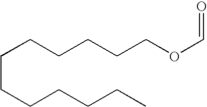 | CH₃ | H |
| ALM-42 | 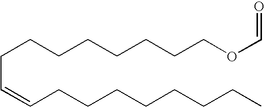 | CH₃ | H |
| ALM-43 | 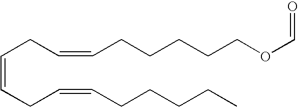 | CH₃ | H |
| ALM-44 | 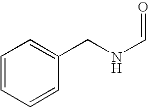 | CH₃ | H |
| ALM-45 | 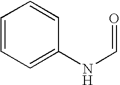 | CH₃ | H |
| ALM-46 | 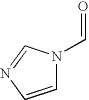 | CH₃ | H |
| ALM-47 | 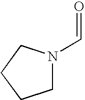 | CH₃ | H |
| ALM-48 | 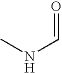 | CH₃ | H |
| ALM-49 | 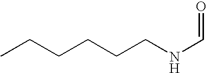 | CH₃ | H |
| ALM-50 | 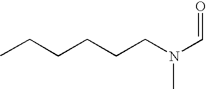 | CH₃ | H |
| ALM-51 | 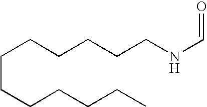 | CH₃ | H |
| ALM-52 | 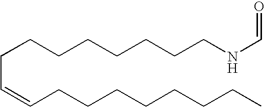 | CH₃ | H |
| ALM-53 | 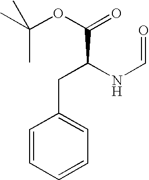 | CH₃ | H |
| ALM-54 | 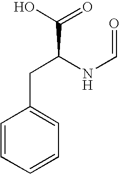 | CH₃ | H |
| ALM-55 | 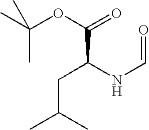 | CH₃ | H |
| ALM-56 | 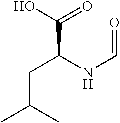 | CH₃ | H |
| ALM-57 | 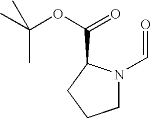 | CH₃ | H |
| ALM-58 | 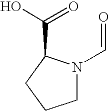 | CH₃ | H |
| ALM-59 | 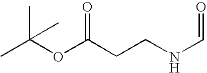 | CH₃ | H |
| ALM-60 | 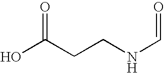 | CH₃ | H |
| ALM-61 | 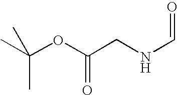 | CH₃ | H |
| ALM-62 | 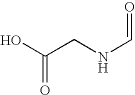 | CH₃ | H |
| ALM-63 | 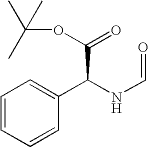 | CH₃ | H |
| ALM-64 | 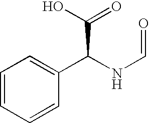 | CH₃ | H |
| ALM-65 | 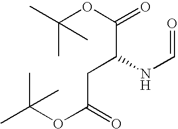 | CH₃ | H |
| ALM-66 | 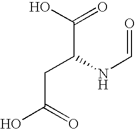 | CH₃ | H |
| ALM-67 | 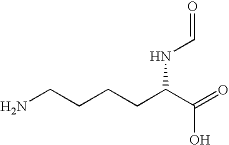 | CH₃ | H |
| ALM-68 | —CO₂CH₃ | H | H |
| ALM-69 | 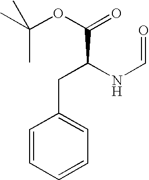 | H | H |
| ALM-70 | 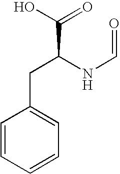 | H | H |
| ALM-71 | —CO₂CH₃ | CH₃ | CH₃ |
| ALM-72 | —CO₂H | CH₃ | CH₃ |
| ALM-73 | 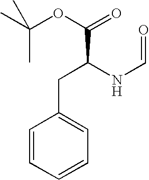 | CH₃ | CH₃ |
| ALM-74 | 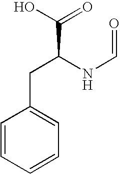 | CH₃ | CH₃ |

Class C

| Compound No. | Structure |
|---|---|
| ALM-75 | 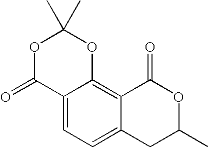 |

¹All compounds are racemic at C-3 position unless otherwise stated
²All compounds have been characterised by ¹H NMR and/or ¹³C NMR and/or MS.
³(3S)-enantiomer
⁴(3R)-enantiomer
⁵All compounds have been characterised by ¹H NMR and/or ¹³C NMR and/or MS.
⁶All compounds are racemic at C-3 position unless otherwise stated

It should be noted that the compound labelled as ALM-54 is a racemic form of ochratoxin B (labelled as compound ALM-32).

The anti-proliferative effect of the compounds as shown in Table 1.1 against certain cancerous cell lines is detailed in Table 1.2 below. The anti-proliferative effect of the compounds is exemplified against the following cancer cell lines: SkMel28, MalMe3M, MCF-7, and MDA-MB-468.

TABLE 1.2
Characterization of Anti-Proliferative Activity of
Compounds on Neoplastic cell lines.

IC 50 (μM)

| | | | | | |
|---|---|---|---|---|---|
| Compound | Molecular | | | | MDA- |
| No. | Weight | SkMel28 | MalMe3M | MCF-7 | MB-468 |

Class A:

| | | | | | |
|---|---|---|---|---|---|
| ALM-1 | 238.2 | 717.8 | 713.7 | 629.7 | 306.5 |
| ALM-2 | 502.7 | >397 | >397 | >397 | >397 |
| ALM-3 | 500.7 | >399 | >399 | 149.8 | 99.9 |
| ALM-4 | 498.7 | >401 | 320.8 | 60.2 | 90.2 |
| ALM-5 | 498.7 | >401 | 150.4 | 20.0 | 50.1 |
| ALM-6 | 546.8 | NT | NT | NT | NT |
| ALM-7 | 222.2 | >900 | 625.6 | 432.0 | 486.0 |
| ALM-8 | 208.2 | 725.3 | 662.8 | 427.5 | 427.5 |
| ALM-9 | 178.2 | 821.3 | >960 | 946.2 | 283.4 |
| ALM-10 | 178.2 | 960.6 | 360.2 | 595.6 | 317 |
| ALM-11 | 178.2 | 960.6 | >960 | 960.6 | 427.5 |
| ALM-12 | 192.2 | >1040 | >1040 | >1040 | 582.7 |
| ALM-13 | 192.2 | >1040 | 822.1 | >1040 | 1040.6 |
| ALM-14 | 192.2 | >1040 | >1040 | 1040.6 | >1040.6 |
| ALM-15 | 220.2 | 622.2 | 613.1 | 613.1 | 345.1 |
| ALM-16 | 250.2 | 359.7 | 455.6 | 295.8 | 239.8 |
| ALM-17 | 442.6 | >451 | >451 | >451 | >451 |
| ALM-18 | 440.6 | >453 | >453 | >453 | >453 |
| ALM-19 | 438.6 | >455 | >455 | >455 | >455 |
| ALM-20 | 438.6 | >455 | >455 | 341.9 | >455 |
| ALM-21 | 486.7 | NT | NT | NT | NT |
| ALM-22 | 196.2 | 96.8 | 96.8 | 173.3 | 61.2 |
| ALM-23 | 196.2 | >1019 | >1019 | >1019 | 468.9 |
| ALM-24 | 257.1 | 295.6 | 248.9 | 260.6 | 171.1 |
| ALM-25 | 304.1 | 78.9 | 75.6 | 72.3 | 72.3 |
| ALM-26 | 304.1 | 164.4 | 167.7 | 328.8 | 263.1 |
| ALM-27 | 430.0 | 76.8 | 109.3 | 116.3 | 48.8 |
| ALM-28 | 318.1 | 282.9 | 484.2 | 594.1 | 210.6 |
| ALM-29 | 220.2 | 363.3 | 340.6 | 340.6 | 340.6 |
| ALM-30 | 268.2 | 178.9 | 141.7 | 130.5 | 137.9 |
| ALM-75 | 262.0 | >761.6 | >761.6 | >761.6 | >761.6 |

Class B:

| | | | | | |
|---|---|---|---|---|---|
| ALM-32 | 369.4 | 21.6 | 13.5 | 18.9 | 18.9 |
| ALM-33 | 222.2 | >900 | >900 | >900 | 585.1 |
| ALM-34 | 236.2 | >846.7 | >846.7 | >846.7 | 359.9 |
| ALM-35 | 370.4 | 539.9 | 440.1 | 345.6 | 356.4 |
| ALM-36 | 312.3 | 176.1 | 105.7 | 102.5 | 99.3 |
| ALM-37 | 298.3 | 331.9 | 308.4 | 284.9 | 181.0 |
| ALM-38 | 304.3 | 131.4 | 131.4 | 98.6 | 131.4 |
| ALM-39 | 278.3 | 359.2 | 344.9 | 391.7 | 305.4 |
| ALM-40 | 306.4 | 163.2 | 114.2 | 65.3 | 42.4 |
| ALM-41 | 390.5 | 256.1 | 332.9 | 192.1 | 166.4 |
| ALM-42 | 472.7 | >423.1 | >423.1 | >423.1 | 317.3 |
| ALM-43 | 468.6 | 21.3 | 23.5 | 2.3 | 4.3 |
| ALM-44 | 311.3 | 269.8 | 128.5 | 86.7 | 41.8 |
| ALM-45 | 297.3 | 171.5 | 33.6 | 26.9 | 10.1 |
| ALM-46 | 272.3 | >734.5 | >734.5 | >734.5 | 73.4 |
| ALM-47 | 275.3 | >726.5 | >726.5 | >726.5 | 490.4 |
| ALM-48 | 235.2 | 501.7 | 191.3 | 267.8 | 289.1 |
| ALM-49 | 305.4 | 104.8 | 65.5 | 26.2 | 3.3 |
| ALM-50 | 319.4 | 140.9 | 109.6 | 90.8 | 59.5 |
| ALM-51 | 389.5 | 33.4 | 25.7 | 12.8 | 10.3 |
| ALM-52 | 471.7 | 243.8 | 52.9 | 21.2 | 12.7 |
| ALM-53 | 425.5 | 70.5 | 11.7 | 9.4 | 2.3 |
| ALM-54 | 369.4 | 46.0 | 13.5 | 16.2 | 10.8 |
| ALM-55 | 391.5 | 79.2 | 22.9 | 43.4 | 12.8 |
| ALM-56 | 335.4 | >596 | 485.9 | 152.1 | 113.1 |
| ALM-57 | 375.4 | >532 | 343.6 | >532 | 309.0 |
| ALM-58 | 319.3 | >626 | >626 | >626 | 585.6 |
| ALM-59 | 349.4 | 186.0 | 103.0 | 68.7 | 57.2 |
| ALM-60 | 293.3 | >681 | 289.8 | 136.4 | 221.6 |
| ALM-61 | 335.4 | 339.9 | 152.1 | 163.9 | 137.1 |
| ALM-62 | 279.2 | >716 | >716 | >716 | >716 |
| ALM-63 | 411.4 | 157.9 | 48.6 | 77.8 | 48.6 |
| ALM-64 | 355.3 | >562 | >562 | 197.0 | 118.2 |
| ALM-65 | 449.5 | 102.3 | 66.7 | 20 | 17.8 |
| ALM-66 | 337.3 | >592 | >592 | >592 | >592 |
| ALM-67 | 350.4 | >570 | >570 | >570 | >570 |
| ALM-68 | 222.2 | 562.5 | 517.5 | 337.5 | 270.0 |
| ALM-69 | 411 | 72.9 | 21.9 | 18.2 | 14.6 |
| ALM-70 | 355 | 42.2 | 18.3 | 8.4 | 1.4 |
| ALM-71 | 250.2 | >799 | >799 | 399.7 | 335.7 |
| ALM-72 | 236.2 | >846 | >846 | >846 | >846 |
| ALM-73 | 439 | 13.7 | 9.1 | 14.8 | 9.1 |
| ALM-74 | 383 | 11.7 | 7.8 | 2.21 | 1.83 |

The particularly preferred compounds of the present

invention for use in therapy or diagnosis are shown in Table 1.3 which also details the anti-proliferative effect of these compounds against cancer cell lines SkMel28, MalMe3M, MCF-7, and MDA-MB-468.

TABLE 1.3
List of Preferred Compounds

| | | IC 50 (μM) | | | |
|---|---|---|---|---|---|
| Compound | Molecular | | | | MDA-MB- |
| No. | Weight | SkMel28 | MalMe3M | MCF-7 | 468 |
| ALM-9 | 178.2 | 821.3 | >960 | 946.2 | 283.4 |
| ALM-10 | 178.2 | 960.6 | 360.2 | 595.6 | 317 |
| ALM-11 | 178.2 | 960.6 | >960 | 960.6 | 427.5 |
| ALM-22 | 196.2 | 96.8 | 96.8 | 173.3 | 61.2 |
| ALM-25 | 304.1 | 78.9 | 75.6 | 72.3 | 72.3 |
| ALM-32 | 369.4 | 21.6 | 13.5 | 18.9 | 18.9 |
| ALM-45 | 297.3 | 171.5 | 33.6 | 26.9 | 10.1 |
| ALM-53 | 425.5 | 70.5 | 11.7 | 9.4 | 2.3 |
| ALM-54 | 369.4 | 46.0 | 13.5 | 16.2 | 10.8 |
| ALM-55 | 391.5 | 79.2 | 22.9 | 43.4 | 12.8 |
| ALM-65 | 449.5 | 102.3 | 66.7 | 20 | 17.8 |
| ALM-69 | 411 | 72.9 | 21.9 | 18.2 | 14.6 |
| ALM-70 | 355 | 42.2 | 18.3 | 8.4 | 1.4 |
| ALM-73 | 439 | 13.7 | 9.1 | 14.8 | 9.1 |
| ALM-74 | 383 | 11.7 | 7.8 | 2.21 | 1.83 |
| ALM-43 | 468.6 | 21.3 | 23.5 | 2.3 | 4.3 |
| ALM-49 | 305.4 | 104.8 | 65.5 | 26.2 | 3.3 |
| ALM-51 | 389.5 | 33.4 | 25.7 | 12.8 | 10.3 |
| ALM-52 | 471.7 | 243.8 | 52.9 | 21.2 | 12.7 |

The anti-proliferative effect of three compounds of the present invention against several different cancel cell lines in detailed in Table 1.4 below.

TABLE 1.4
List of Preferred Compounds - Additional
Cancer Cell Types

| | IC50 (μM) | | |
|---|---|---|---|
| Cell Line. | ALM-9 | ALM-54 | ALM-43 |
| MCF-7 | 946.2 | 16.2 | 2.3 |
| MDA-MB-468 | 283.4 | 10.8 | 4.3 |
| ZR-75-1 | 280.6 | 0.13 | 6.4 |
| SkMel28 | 821.3 | 46.0 | 21.3 |
| MalMe3M | >960 | 13.5 | 23.5 |
| RKO | 493.8 | 2.7 | 10.7 |
| HT29 | 561.2 | 1.3 | 18.7 |
| HCT−/− | >561.2 | 10.8 | 42.7 |
| HCT+/+ | >561.2 | 16.3 | 21.3 |
| H23 | >561.2 | 2.0 | 2.1 |
| H157 | >561.2 | | NT |
| PC3 | 420.9 | 5.4 | 9.6 |
| LNCAP | >561.2 | 4.7 | 10.7 |
| PNT2 | 420.9 | 5.4 | 5.3 |

The ability of the preferred compounds to induce apoptosis in representative breast cancer (MDA-MB-468 and MCF-7 cell lines) and melanoma cell lines (MalMe3, SkMe128) was demonstrated by analysis of flow cytometry profiles of the cells following exposure to these drugs. The level of apoptosis detected by this technique is represented by the percentage of cells detected in the sub G0/G1 peak as shown in Table 1.5. In addition, treatment of these cancer cell lines with exemplars of the present invention was shown to (effect cleavage of the caspase substrate PARP in the indicated cancer cell lines (see FIG. 8).

TABLE 1.5
List of Preferred Compounds - Percentage cells
in pre G0 phase of cell cycle following treatment with
IC70 concentration of compound

| | % Cells in Pre G0 |
|---|---|
| Compound No. | phase of Cell Cycle |
| ALM-9 | 30.4 |
| ALM-22 | 16.5 |
| ALM-25 | 37.0 |
| ALM-32 | NT |
| ALM-45 | 6.4 |
| ALM-53 | 8.9 |
| ALM-54 | 12.8 |
| ALM-55 | 3.4 |
| ALM-65 | 8.5 |
| ALM-43 | 11.8 |
| ALM-49 | 14.1 |
| ALM-51 | 20.6 |
| ALM-52 | 19.8 |
| ALM-69 | 14.4 |
| ALM-70 | 32.7 |
| ALM-73 | 25.3 |
| ALM-74 | 34.4 |

The synergy of the combination of compound ALM-43 with etoposide, irinotecan, cisplatin and doxorubicin against proliferation of cancer cell line MDA-MB-468 is demonstrated in Table 1.6 below. Evidence of synergy was determined by calculation of the combination index using Calcusyn software.

TABLE 1.6
The Combination Index of Etoposide and
Compound ALM-43 at differing Concentrations

| | | Combination Index | | | |
|---|---|---|---|---|---|
| | | ALM- | ALM- | ALM- | ALM- |
| | M | 43 | 43 | 43 | 43 |
| Agent | mol/L | 0.1 μg/ml | 0.5 μg/ml | 1.0 μg/ml | 5.0 μg/ml |
| Etoposide | 1 × 10⁻³ | 1.001 | 0.341 | 0.443 | No |
| | 1 × 10⁻⁴ | 0.399 | 0.289 | 0.418 | 0.474 |
| | 1 × 10⁻⁵ | No | No | No | No |
| | 1 × 10⁻⁶ | No | 0.865 | No | No |
| | 1 × 10⁻⁷ | 0.473 | 0.305 | No | No |
| | 1 × 10⁻⁸ | 0.181 | 0.137 | No | No |
| | 1 × 10⁻⁹ | 0.090 | 0.319 | No | No |
| | 1 × 10⁻¹⁰ | 0.083 | 0.515 | No | No |
| Cisplatin | 1 × 10⁻² | 0.063 | 0.003 | 0.007 | 0.038 |
| | 1 × 10⁻³ | No | No | No | No |
| | 1 × 10⁻⁴ | No | No | No | No |
| | 1 × 10⁻⁵ | No | No | No | No |
| | 1 × 10⁻⁶ | No | No | No | No |
| | 1 × 10⁻⁷ | 0.636 | 0.612 | No | No |
| | 1 × 10⁻⁸ | 0.199 | No | No | No |
| | 1 × 10⁻⁹ | 0.054 | 0.536 | No | No |
| Irinotecan | 1 × 10⁻³ | 0.088 | 0.230 | 0.438 | 0.307 |
| | 1 × 10⁻⁴ | 0.019 | 0.005 | 0.040 | 0.113 |
| | 1 × 10⁻⁵ | No | No | No | No |
| | 1 × 10⁻⁶ | No | No | No | No |
| | 1 × 10⁻⁷ | No | No | No | No |
| | 1 × 10⁻⁸ | 0.514 | 0.233 | No | No |
| | 1 × 10⁻⁹ | 0.701 | 0.628 | No | No |
| | 1 × 10⁻¹¹ | 0.172 | 0.484 | No | No |
| Doxorubicin | 1 × 10⁻⁴ | No | No | No | No |
| | 1 × 10⁻⁵ | 0.385 | 0.700 | 0.278 | 0.457 |
| | 1 × 10⁻⁶ | 0.001 | 0.003 | 0.004 | 0.028 |
| | 1 × 10⁻⁷ | 0.378 | 0.435 | 0.300 | 0.617 |
| | 1 × 10⁻⁸ | 0.642 | 1.116 | 0.469 | No |
| | 1 × 10⁻⁹ | No | No | 0.548 | No |
| | 1 × 10⁻¹⁰ | No | No | No | No |
| | 1 × 10⁻¹¹ | No | No | No | No |

The synergy of the combination of compound ALM-49 with etoposide, irinotecan, cisplatin and doxorubicin against proliferation of cancer cell line MDA-MB-468 is demonstrated in Table 1.7.

TABLE 1.7
The Combination Index of Etoposide and ALM-49
at differing Concentrations

| | | Combination Index | | | |
|---|---|---|---|---|---|
| | | ALM- | ALM- | ALM- | ALM- |
| | M | 49 | 49 | 49 | 49 |
| Agent | mol/L | 0.1 μg/ml | 0.5 μg/ml | 1.0 μg/ml | 5.0 μg/ml |
| Etoposide | 1 × 10⁻³ | No | No | 0.567 | 0.763 |
| | 1 × 10⁻⁴ | 0.385 | 0.234 | 0.210 | 0.175 |
| | 1 × 10⁻⁵ | 1.184 | 0.659 | 1.065 | 0.941 |
| | 1 × 10⁻⁶ | No | 0.806 | No | No |
| | 1 × 10⁻⁷ | 1.167 | 0.78 | No | No |
| | 1 × 10⁻⁸ | No | No | No | No |
| | 1 × 10⁻⁹ | 0.836 | No | No | No |
| | 1 × 10⁻¹⁰ | 0.344 | No | No | No |
| Cisplatin | 1 × 10⁻² | 0.003 | 0.002 | 0.009 | 0.051 |
| | 1 × 10⁻³ | No | No | No | No |
| | 1 × 10⁻⁴ | No | No | No | No |
| | 1 × 10⁻⁵ | No | No | No | No |
| | 1 × 10⁻⁶ | No | No | No | No |
| | 1 × 10⁻⁷ | 0.625 | No | No | No |
| | 1 × 10⁻⁸ | 0.119 | No | No | No |
| | 1 × 10⁻⁹ | 0.487 | No | No | No |
| Irinotecan | 1 × 10⁻³ | No | 0.606 | 0.715 | 0.557 |
| | 1 × 10⁻⁴ | 0.029 | 0.019 | 0042 | 0.042 |
| | 1 × 10⁻⁵ | No | No | No | No |
| | 1 × 10⁻⁶ | No | No | No | No |
| | 1 × 10⁻⁷ | No | No | No | No |
| | 1 × 10⁻⁸ | No | No | No | No |
| | 1 × 10⁻⁹ | No | No | No | No |
| | 1 × 10⁻¹⁰ | No | No | No | No |
| Doxorubicin | 1 × 10⁻⁴ | No | No | No | 0.968 |
| | 1 × 10⁻⁵ | 0.410 | 0.436 | 0.438 | 0.720 |
| | 1 × 10⁻⁶ | 0.000 | 0.001 | 0.003 | 0.019 |
| | 1 × 10⁻⁷ | 0.462 | 0.567 | 0.847 | No |
| | 1 × 10⁻⁸ | 0.300 | 0.632 | 1.018 | No |
| | 1 × 10⁻⁹ | 0.826 | No | No | No |
| | 1 × 10⁻¹⁰ | No | 0.705 | 0.641 | No |
| | 1 × 10⁻¹¹ | 0.314 | No | No | No |

The synergy of the combination of compound ALM-55 with etoposide, irinotecan, cisplatin and doxorubicin against proliferation of cancer cell line MDA-MB-468 is demonstrated in Table 1.8.

TABLE 1.8
The Combination Index of Etoposide and
Compound ALM-55 at differing Concentrations

| | | Combination Index | | | |
|---|---|---|---|---|---|
| | | ALM- | ALM- | ALM- | ALM- |
| | M | 55 | 55 | 55 | 55 |
| Agent | mol/L | 0.1 μg/ml | 0.5 μg/ml | 1.0 μg/ml | 5.0 μg/ml |
| Etoposide | 1 × 10⁻³ | 0.800 | 0.609 | 1.097 | 0.920 |
| | 1 × 10⁻⁴ | 0.312 | 0.142 | 0.473 | 0.335 |
| | 1 × 10⁻⁵ | No | 1.136 | No | No |
| | 1 × 10⁻⁶ | No | No | No | No |
| | 1 × 10⁻⁷ | No | No | No | No |
| | 1 × 10⁻⁸ | 0.414 | 0.910 | No | No |
| | 1 × 10⁻⁹ | 0.318 | 0.829 | No | No |
| | 1 × 10⁻¹⁰ | 0.267 | 0.494 | No | No |
| Cisplatin | 1 × 10⁻² | 0.003 | 0.009 | 0.023 | 0.11 |
| | 1 × 10⁻³ | 0.031 | No | No | No |
| | 1 × 10⁻⁴ | No | No | No | No |
| | 1 × 10⁻⁵ | No | No | No | No |
| | 1 × 10⁻⁶ | No | No | No | No |
| | 1 × 10⁻⁷ | 0.431 | No | No | No |
| | 1 × 10⁻⁸ | 0.155 | No | No | No |
| | 1 × 10⁻⁹ | 0.093 | 0.531 | No | No |
| Irinotecan | 1 × 10⁻³ | 1.133 | 0.604 | 0.531 | 0.611 |
| | 1 × 10⁻⁴ | 0.205 | 0.136 | 0.053 | 0.128 |
| | 1 × 10⁻⁵ | No | 0.682 | No | No |
| | 1 × 10⁻⁶ | No | No | No | No |
| | 1 × 10⁻⁷ | No | No | No | No |
| | 1 × 10⁻⁸ | No | 0.500 | No | No |
| | 1 × 10⁻⁹ | No | No | No | No |
| | 1 × 10⁻¹⁰ | 0.061 | No | No | No |
| Doxorubicin | 1 × 10⁻⁴ | No | No | No | No |
| | 1 × 10⁻⁵ | 0.284 | No | 0.344 | 0.969 |
| | 1 × 10⁻⁶ | 0.001 | 0.609 | 0.006 | 0.037 |
| | 1 × 10⁻⁷ | 0.329 | 0.004 | 0.934 | No |
| | 1 × 10⁻⁸ | 0.044 | 0.551 | 0.883 | No |
| | 1 × 10⁻⁹ | 0.022 | 0.636 | No | No |
| | 1 × 10⁻¹⁰ | 0.02 | No | No | No |
| | 1 × 10⁻¹¹ | 0.023 | No | No | No |

The synergy of the combination of compound ALM-65 with etoposide, irinotecan, cisplatin and doxorubicin against proliferation of cancer cell line MDA-MB-469 is demonstrated in Table 1.9.

TABLE 1.9
The Combination Index of Etoposide and
Compound ALM-65 at differing Concentrations

| | | Combination Index | | | |
|---|---|---|---|---|---|
| | | ALM- | ALM- | ALM- | ALM- |
| | M | 65 | 65 | 65 | 65 |
| Agent | mol/L | 0.1 μg/ml | 0.5 μg/ml | 1.0 μg/ml | 5.0 μg/ml |
| Etoposide | 1 × 10⁻³ | 0.423 | 0.350 | 0.372 | 0.394 |
| | 1 × 10⁻⁴ | 0.884 | 0.587 | 0.503 | No |
| | 1 × 10⁻⁵ | No | No | No | No |
| | 1 × 10⁻⁶ | No | No | No | No |
| | 1 × 10⁻⁷ | No | No | No | No |
| | 1 × 10⁻⁸ | No | No | No | 0.469 |
| | 1 × 10⁻⁹ | 0.720 | No | No | 0.355 |
| | 1 × 10⁻¹⁰ | No | No | No | 0.180 |
| Cisplatin | 1 × 10⁻² | 0.006 | 0.012 | 0.011 | 0.04 |
| | 1 × 10⁻³ | No | No | No | No |
| | 1 × 10⁻⁴ | No | No | No | No |
| | 1 × 10⁻⁵ | No | No | No | No |
| | 1 × 10⁻⁶ | No | No | No | 0.206 |
| | 1 × 10⁻⁷ | No | 0.518 | 0.614 | 0.182 |
| | 1 × 10⁻⁸ | No | 0.051 | 0.081 | 0.171 |
| | 1 × 10⁻⁹ | No | No | 0.164 | 0.182 |
| Irinotecan | 1 × 10⁻³ | 0.268 | 0.154 | 0.393 | 0.181 |
| | 1 × 10⁻⁴ | 0.170 | 0.657 | 0.046 | 0.050 |
| | 1 × 10⁻⁵ | No | No | No | No |
| | 1 × 10⁻⁶ | No | No | No | No |
| | 1 × 10⁻⁷ | No | No | No | 0.929 |
| | 1 × 10⁻⁸ | No | No | No | 0.784 |
| | 1 × 10⁻⁹ | 0.742 | 0.983 | No | 0.311 |
| | 1 × 10⁻¹⁰ | 0.026 | No | No | 0.250 |
| Doxorubicin | 1 × 10⁻⁴ | No | No | 0.325 | 0..412 |
| | 1 × 10⁻⁵ | No | 1.009 | 0.052 | 0.048 |
| | 1 × 10⁻⁶ | 0.672 | 0.000 | 0.000 | 0.001 |
| | 1 × 10⁻⁷ | 1.013 | 0.561 | 0.032 | 0.047 |
| | 1 × 10⁻⁸ | 0.862 | 0.599 | 0.089 | 0.162 |
| | 1 × 10⁻⁹ | No | 0.803 | 0.175 | 0.164 |
| | 1 × 10⁻¹⁰ | No | No | 0.527 | 0.274 |
| | 1 × 10⁻¹¹ | No | No | 0.527 | 0.485 |

The synergy of the combination of compound ALM-74 with etoposide, irinotecan, cisplatin and doxorubicin against proliferation of cancer cell line MDA-MB-468 is demonstrated in Table 1.10.

TABLE 1.10
The Combination Index of Etoposide and
Compound ALM-74 at differing Concentrations

| | | Combination Index | | | |
|---|---|---|---|---|---|
| | | ALM- | ALM- | ALM- | ALM- |
| | M | 74 | 74 | 74 | 74 |
| Agent | mol/L | 0.1 μg/ml | 0.5 μg/ml | 1.0 μg/ml | 5.0 μg/ml |
| Etoposide | 1 × 10⁻³ | 0.606 | 0.554 | 0.536 | 0.294 |
| | 1 × 10⁻⁴ | 0.355 | 0.402 | 0.307 | 0.349 |
| | 1 × 10⁻⁵ | No | 0.613 | 0.031 | 0.053 |
| | 1 × 10⁻⁶ | No | No | 0.015 | 0.200 |
| | 1 × 10⁻⁷ | No | No | 0.016 | 0.231 |
| | 1 × 10⁻⁸ | No | No | 0.016 | 0.261 |
| | 1 × 10⁻⁹ | No | No | 0.019 | 0.251 |
| | 1 × 10⁻¹⁰ | 0.724 | No | 0.022 | 0.265 |
| Cisplatin | 1 × 10⁻² | 0.006 | 0.008 | 0.082 | 0.130 |
| | 1 × 10⁻³ | No | No | 0.039 | 0.346 |
| | 1 × 10⁻⁴ | No | No | 0.061 | 1.031 |
| | 1 × 10⁻⁵ | No | No | 0.069 | 0.778 |
| | 1 × 10⁻⁶ | No | No | 0.083 | No |
| | 1 × 10⁻⁷ | No | No | 0.092 | No |
| | 1 × 10⁻⁸ | No | No | 0.091 | No |
| | 1 × 10⁻⁹ | 0.895 | No | 0.090 | No |
| Irinotecan | 1 × 10⁻³ | 0.916 | 0.121 | No | No |
| | 1 × 10⁻⁴ | 0.189 | 0.302 | 0.051 | 0.051 |
| | 1 × 10⁻⁵ | No | No | 0.025 | 0.616 |
| | 1 × 10⁻⁶ | 0.518 | No | 0.055 | 0.589 |
| | 1 × 10⁻⁷ | 0.067 | No | 0.054 | 0.726 |
| | 1 × 10⁻⁸ | 0.046 | No | 0.066 | 0.895 |
| | 1 × 10⁻⁹ | 0.049 | No | 0.078 | 0.973 |
| | 1 × 10⁻¹⁰ | 0.22 | No | 0.086 | 0.720 |
| Doxorubicin | 1 × 10⁻⁴ | No | 0.277 | No | No |
| | 1 × 10⁻⁵ | 0.001 | 0.007 | 0.376 | 1.069 |
| | 1 × 10⁻⁶ | 0.022 | 0.709 | 0.011 | 0.050 |
| | 1 × 10⁻⁷ | 0.236 | No | 0.025 | No |
| | 1 × 10⁻⁸ | 0.319 | No | 0.026 | No |
| | 1 × 10⁻⁹ | 0.622 | No | 0.050 | No |
| | 1 × 10⁻¹⁰ | 0.411 | No | 0.031 | No |
| | 1 × 10⁻¹¹ | 0.888 | No | 0.029 | 0.991 |

### Synthetic Chemistry Section

The preparation of racemic kigelin (ALM-1) is shown in Scheme 1.

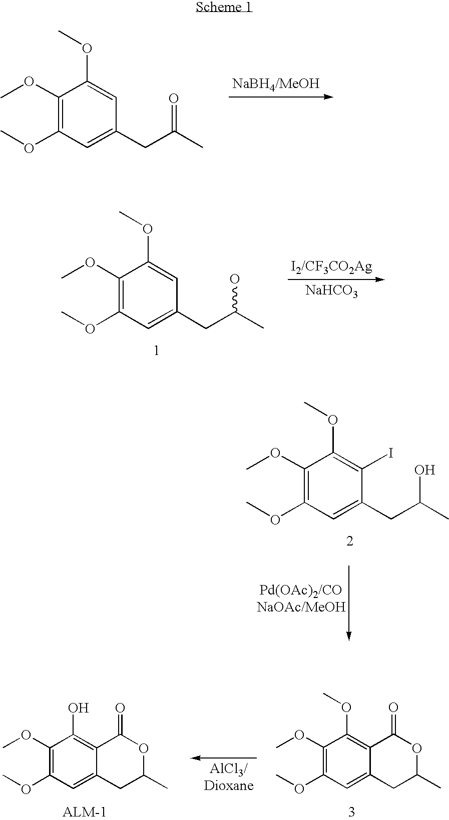

Fatty acid esters of racemic kigelin (compounds ALM-2 to ALM-6) were prepared by the following general method.

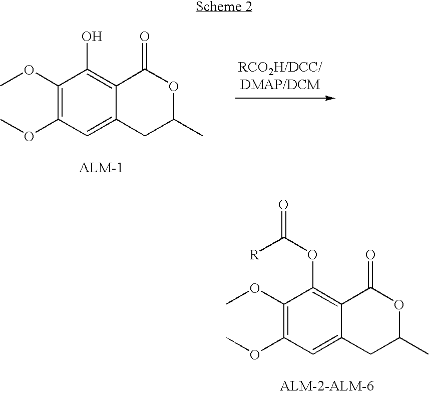

Synthetic route to compounds ALM-7 and ALM-8:

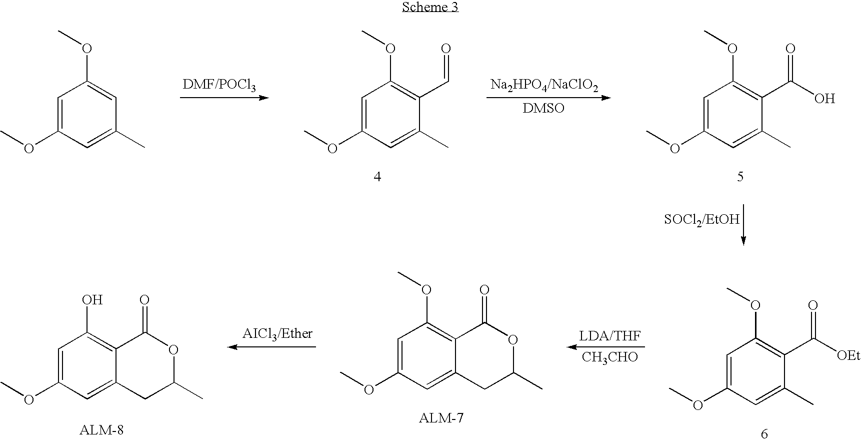

The preparation of mellein and methoxy mellein (racemic, (S) and (R) isomers, compounds ALM-9 to ALM-14) was achieved as shown in scheme 4.

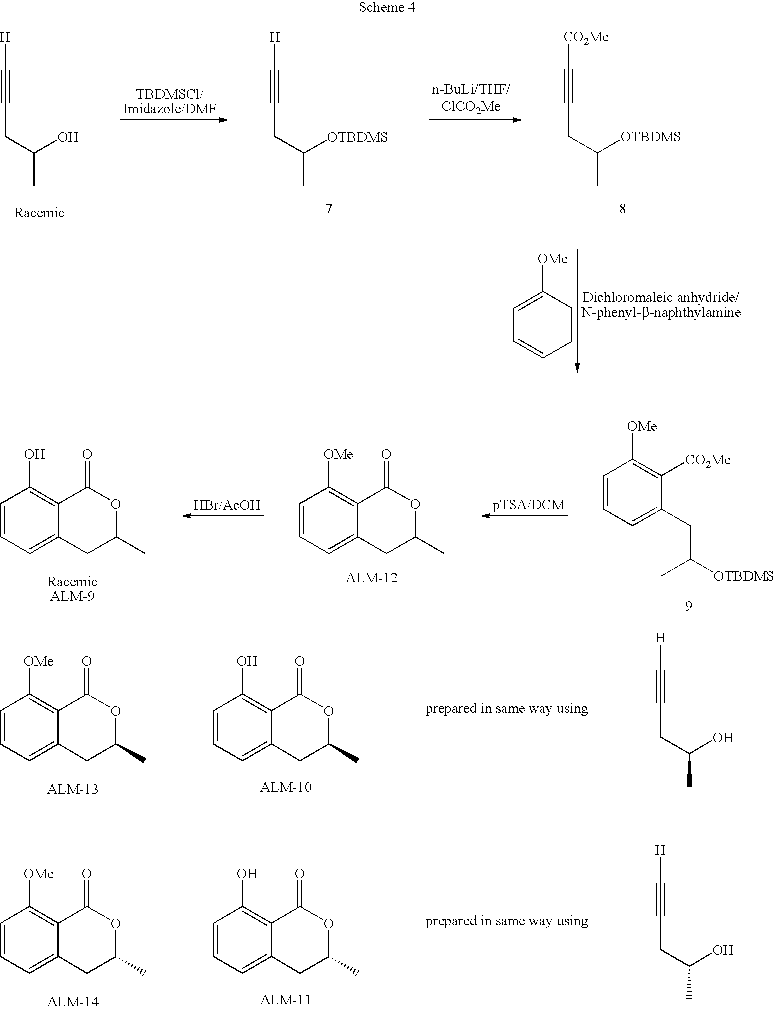

Fatty acid esters of racemic mellein (compounds ALM-17 to ALM-21) were prepared by the following general method.

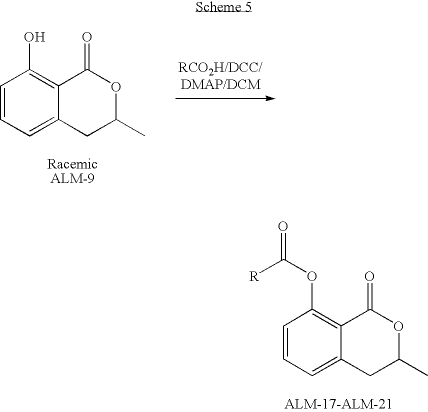

Analogues of racemic mellein (compounds ALM-15, ALM-16 ALM-22 to ALM-30) were all prepared by the following general methods except ALM-24, which was prepared according to M. Gill et al. J. Chem. Soc., Perkin Trans. 1, 2002, 938.

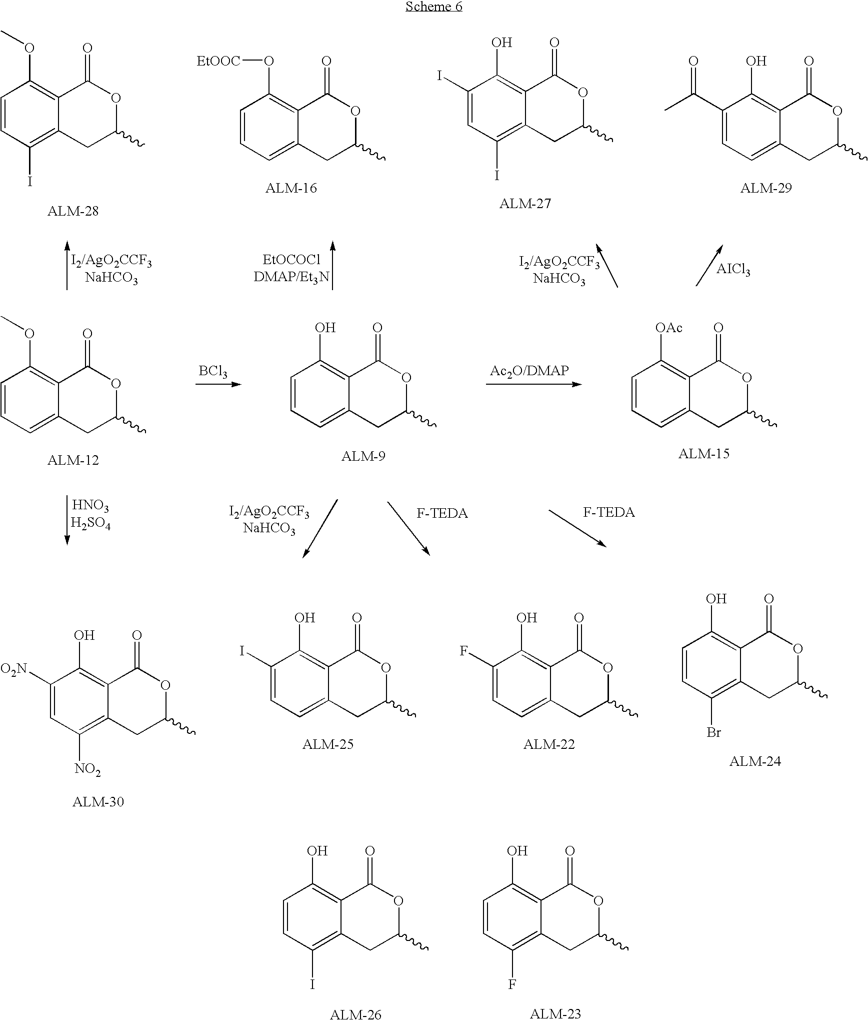

Route 1 to Oclaratoxin acid—from racemic ortho-iodomellein (ALM-25)

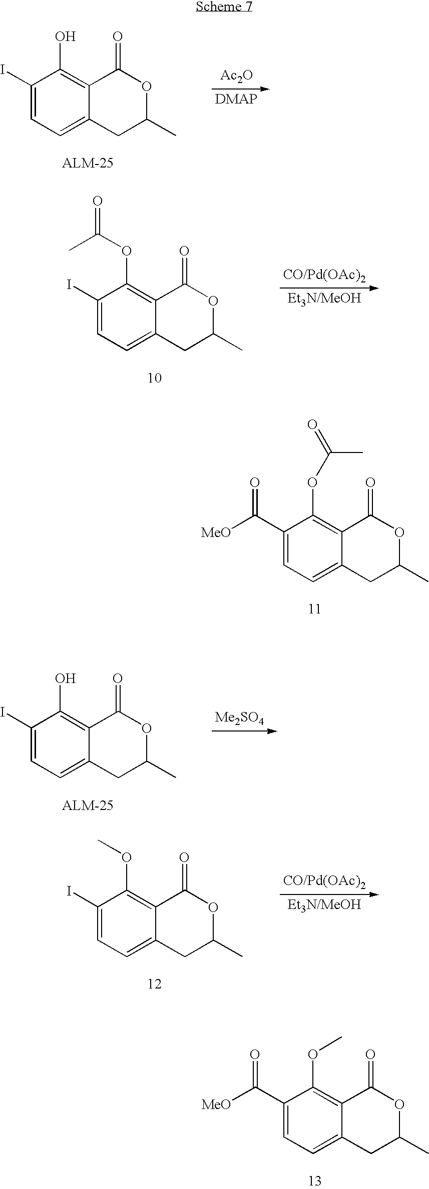

Route 2 to Ochratoxin acid.

Intermediates in scheme 8 were prepared according to literature procedures reported by M. Gill et al., Tetrahedron Asymmetry. 1997, 13, 2153. M. Jounet et al., Tetrahedron Lett. 1998, 39, 6427 and A. Covarrubias-Zú\check{n}iga et al., J. Org. Chem., 1997, 62, 5688.

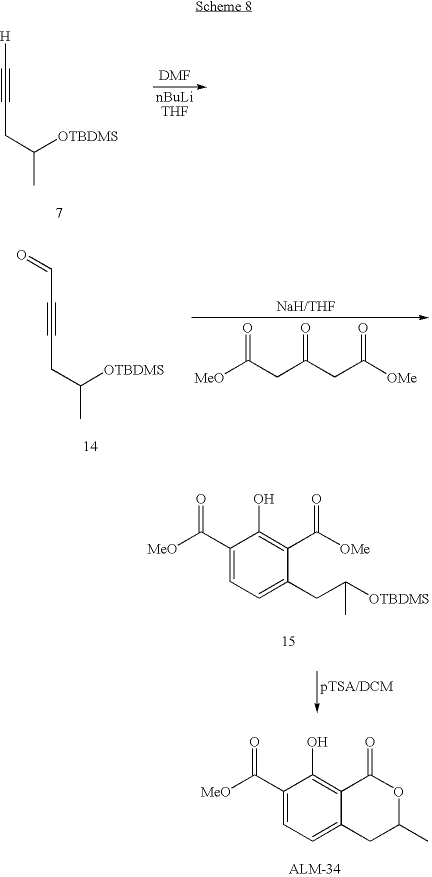

Route 3 to ochratoxin acid.

The compounds ALM-33, ALM-34, ALM-68, ALM-71 and ALM-72 were all prepared as shown in reaction scheme 9.

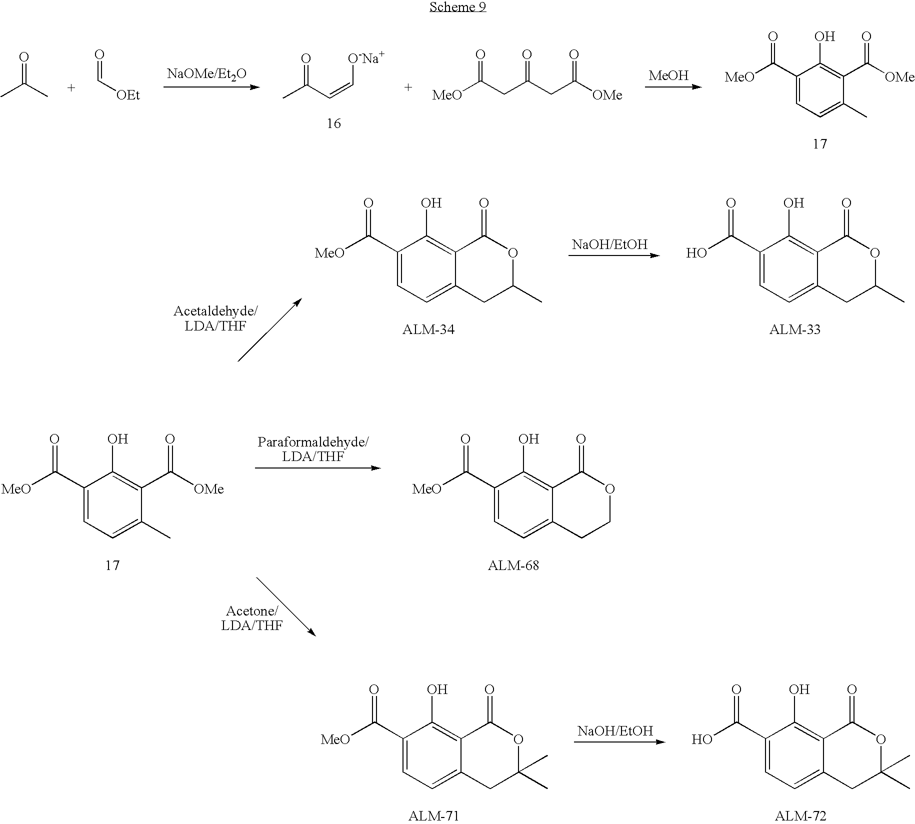

Route to prepare esters of compound ALM-33 (compounds ALM-35 to ALM-43)

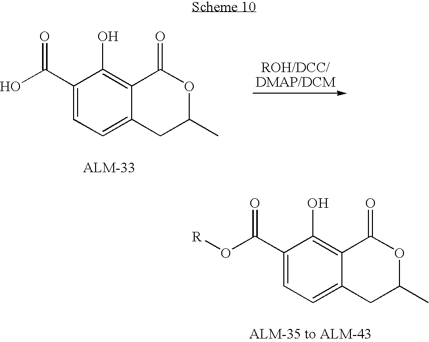

Route to prepare amides of compound ALM-33 (compounds ALM-44 to ALM-52)

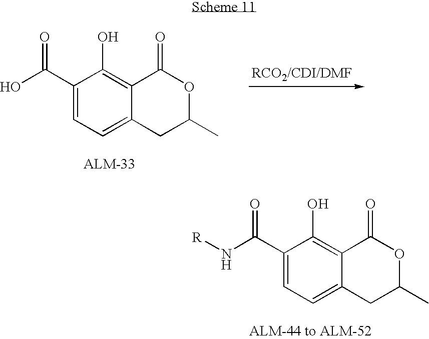

Route to prepare compounds ALM-53 to ALM-67

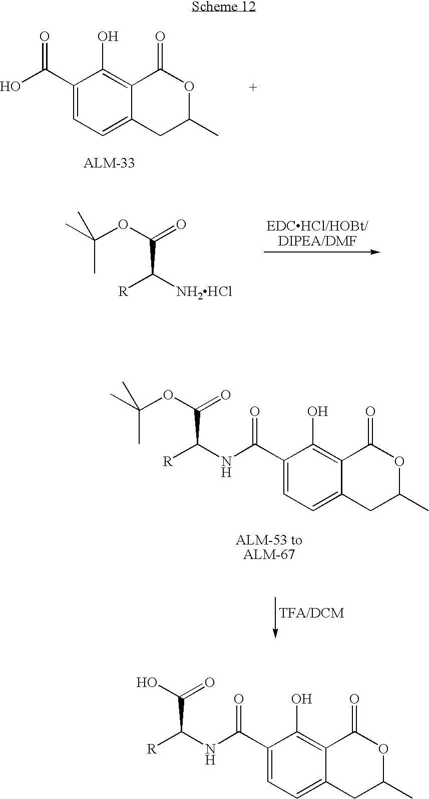

Route to prepare compounds ALM-69 and ALM-70.

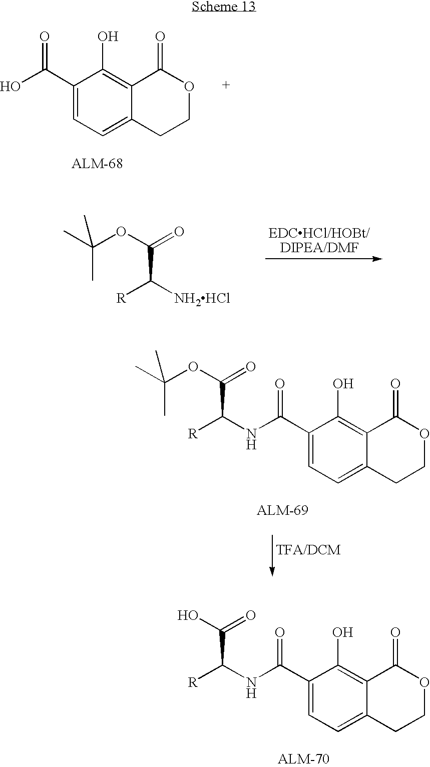

Route to prepare compounds ALM-73 and ALM-74.

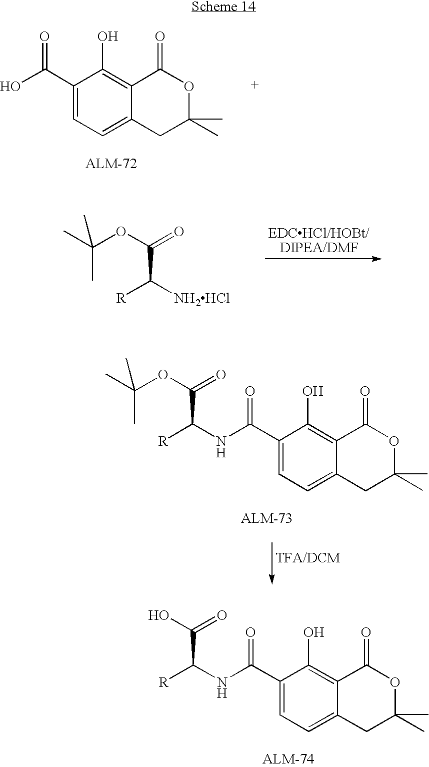

Route to prepare cyclic acetonide compound no ALM-75.

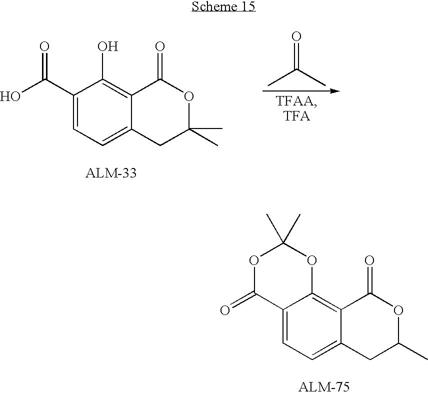

### EXAMPLES

### Preparation of Racemic Kigelin (ALM-1)

### 3,4,5-trimethoxyphenyl propan-2-ol (1)

To a stirred solution of 3,4,5-trimethoxyphenyl acetone (25 g, 0.11 mol, 1 eq) in anhydrous methanol (˜500 mL) at room temperature under nitrogen was added portion wise NaBH₄ (10.5 g, 0.28 mol, 2.5 eq) and then stirred for a further 18 h. The reaction was quenched with saturated NH₄Cl solution (˜500 ml), before the removal of methanol in vacuo. The residue was extracted with DCM and the combined extracts were dried over MgSO₄ filtered and evaporated to dryness in vacuo to afford 25.22 g (˜98%) of the desired compound 3,4,5-trimethoxyphenyl propan-2-ol (1) as a light brown oil.

### 2-Iodo-3,4,5-trimethoxyphenyl propan-2-ol (2)

Solid iodine (29.7 g, 0.12 mol, 1.1 eq) was added portion wise to a stirred suspension of 3,4,5-trimethoxyphenyl propan-2-ol (1) (25.2 g, 0.11 mol, 1 eq), AgOCOCF₃ (30.8 g, 0.14 mol, 1.3 eq) and NaHCO₃ (18.7 g, 0.22 mol, 2.0 eq) in anhydrous DCM (750 ml) under nitrogen at room temperature. The mixture was stirred for a further 1.5 h before filtering through celite. The filtrate was washed with sodium bisulfite solution, separated, dried over MgSO₄ and concentrated in vacuo. The oily residue was purified by column chromatography on silica using hexane/EtOAc to afford 20.2 g (50%) of 2-iodo-3,4,5-trimethoxyphenyl propan-2-ol (2) as a light orange oil.

¹H NMR (CDCl₃) 400 MHz δ6.61 (1H, s), 4.04 (1H, m), 3.85 (3H, s), 3.81 (6H, s), 2.94-2.70 (2H, m), 1.24 (3H, d).

### O-methylkigelin (3)

A solution of 2-iodo-3,4,5-trimethoxyphenyl propan-2-ol (2) (16 g, 45 mmol, 1 eq) in anhydrous MeOH (˜500 mL) together with palladium (II) acetate (3.45 g, 15.4 mmol, 0.34 eq) and NaOAc (15 g, 0.18 mol, 4 eq) were placed in a Parr pressure vessel and exposed to an atmosphere of carbon monoxide at a pressure of 5 Bar and a temperature of 50° C. for 48 h. The reaction mixture was filtered through a celite pad and the solvent removed in vacuo to afford 8.3 g (75%) of O-methylkigelin (3) as a yellow solid.

¹H NMR (CDCl₃) 400 MHz δ6.49 (1H, s), 4.54 (1H, m), 3.97 (3H, s), 3.92 (3H, s), 3.86 (3H, s), 2.82 (2H, m), 1.47 (3H, d).

### Racemic (±)—Kigelin (ALM-1)

Powdered AlCl₃ (3.96 g, 30 mmol, 2.5 eq) was cautiously added to a solution of O-methylkigelin (3) (3 g, 12 mmol, 1.0 eq) in diethyl ether (˜250 mL) and dioxane (˜150 mL) at room temperature under nitrogen before heating to 40° C. After 4 h more AlCl₃ (0.4 g, 3 mmol, 0.25 eq) was added and the reaction mixture heated at 55° C. for 18 h. After cooling to room temperature the reaction mixture was quenched cautiously with water. The mixture was extracted with ether, and combined. The aqueous phase was then back extracted with DCM. Both organic extracts were combined, washed with brine, dried over MgSO₄ and concentrated under reduced pressure. The residue was purified by column chromatography to afford 3.5 g (62% of racemic (±)—kigelin (ALM-1) as an off-white solid.

¹H NMR (CDCl₃) 400 MHz δ6.36 (1H, s), 4.69 (1H, m), 3.92 (3H, s), 3.88 (3H, s), 2.89 (2H, d), 1.52 (3H, d).

¹³C NMR (CDCl₃) 100 MHz δ69.8, 158.5, 156.3, 135.6, 135.4, 102.9, 102.1, 75.8, 60.8, 56.2, 34.8, 20.7.

MS m/z (M−1)=237.

### General Procedure A

### For the Synthesis of Fatty Acids Esters of Racemic Kigelin (Compounds ALM-2 to ALM-6)

To a solution of the fatty acid (100 mg, 1 eq) in DCM under nitrogen were added DCC (1 eq.), DMAP (1 eq) and the reaction mixture cooled to 0° C. Racemic kigelin (ALM-1) (1 eq) was added and the reaction mixture stirred for 2 h at 0° C. The reaction mixture was filtered and the filtrate concentrated in vacuo. The residue was purified by column chromatography using hexane/ether. The product was dissolved in diethyl ether and washed 2M NaOH. The organic phase was separated, dried over MgSO₄ and concentrated in vacuo to afford the fatty acid esters of racemic kigelin.

### Racemic Kigelin Oleyl Ester (ALM-2)

Prepared following general procedure A. Obtained 80 mg (45%).

¹H NMR (CDCl₃) 400 MHz δ6.54 (1H, s), 5.27 (2H, m), 4.50 (1H, m, br), 3.85 (3H, s), 3.73 (3H, s), 2.77 (2H, m), 2.62 (2H, t), 1.95 (4H, d, br), 1.72 (2H, m), 1.38 (3H, d), 1.26-1.19 (20H, m), 0.80 (3H, t).

### Racemic Kigelin Linoleyl Ester (ALM-3)

Prepared following general procedure A. Obtained 55 mg (31%).

¹H NMR (CDCl₃) 400 MHz δ6.54 (1H, s), 5.29 (4H, m), 4.50 (1H, m, br), 3.85 (3H, s), 3.73 (3H, s), 2.79-2.60 (6H, m), 1.98 (4H, m), 1.72 (2H, m), 1.38 (3H, d), 1.32-1.18 (14H, m), 0.80 (3H, t).

### Racemic Kigelin α-Linolenyl Ester (ALM-4)

Prepared following general procedure A. Obtained 68 mg (38%).

¹H NMR (CDCl₃) 400 MHz δ36.61 (1H, s), 5.39 (6H, 4.58 (1H, m, br), 3.92 (3H, s), 3.81 (3H, s), 2.83 (6H, m), 2.69 (2H, t), 2.07 (4H, m), 1.79 (2H, m), 1.47-1.31 (11H, d), 0.98 (3H, t).

### Racemic Kigelin γ-Linolenyl Ester (ALM-5)

Prepared following general procedure A. Obtained 29 mg (16%).

¹H NMR (CDCl₃) 400 MHz δ6.61 (1H, s), 5.47 (6H, m), 4.60 (1H, m, br), 3.93 (3H, s), 3.81 (3H, s), 2.81 (6H, m), 2.71 (2H, t), 2.14 (2H, q), 2.05 (2H, q), 1.82 (2H, m), 1.57-1.23 (11H, m), 0.88 (3H, t).

### Racemic Kigelin Heneicosanoyl Ester (ALM-6)

Prepared following general procedure A. Obtained 78 mg-(47%).

¹H NMR (CDCl₃) 400 MHz δ6.61 (1H, s), 4.58 (1H, s, br), 3.92 (3H, s), 3.81 (3H, s), 2.84 (2H, m), 2.68 (2H, t) 1.78 (2H, m), 1.47-1.26 (37H, m), 0.88 (3H, t).

### Preparation of Compounds ALM-7 and ALM-8

### 2,4-Dimethoxy-6-methyl-benzaldehyde (4)

The Vilsmeler reagent was prepared by the drop wise addition over 15 min of phosphoryl chloride (5.6 mL, 1.2 eq) to a stirred solution of dry DMF (10 mL) under nitrogen at 0° C. The mixture was allowed to warm to room temperature and was then added over a period of ˜30 min to a stirred solution of 2,4-dimethoxy-6-methylbenzene (50 mmol, 7.6 g, 1.0 eq) in 15 mL of dry DMF at 100-110° C. oil bath, under nitrogen atmosphere. Heating and stirring were continued until TLC indicated that the substrate has been consumed (˜1 h). The mixture was poured onto ice-water, made slightly basic (pH˜8) by the addition of aqueous saturated solution of K₂CO₃. The purple solution became yellow and the solid precipitate formed was isolated and dried in a dessicator over P₂O₅ to afford 8.2 g (91%) of 2,4-dimethoxy-6-methyl-benzaldehyde (4) as an off-white solid.

¹H NMR (CDCl₃) 400 MHz δ10.47 (1H, s), 6.32 (2H, s), 3.87 (3H, s), 3.85 (3H, s), 2.58 (3H, s).

¹³C NMR (CDCl₃) 100 MHz δ190.5, 165.1, 164.5, 144.7, 117.3, 108.8, 95.7, 55.7, 55.4, 22.3.

### 2,4-Dimethoxy-6-methyl-benzoic acid (5)

Solid 2,4-dimethoxy-6-methyl-benzaldehyde (4) (0.36 g, 2.0 mmol, 1.0 eq) was added in one portion to solution of NaOH (0.12 g, 3.0 mmol, 1.5 eq) in water (˜5 ml). Solid KMnO₄ (0.316 g, 2.0 mmol, 1 eq) was then added to this mixture portion wise over 10 min whilst heating the water bath at 40° C. The temperature was raised to 50° C. and the reaction heated for a further 15-20 min. The brown precipitate formed was hot filtered through a celite pad and washed with 3 small portions of water. The pale yellow filtrate solution was acidified with 2M HCl to ˜pH1 and the precipitate collected and sucked dry to afford 0.28 g (70%) of 2,4-dimethoxy-6-methyl-benzoic acid (5) as a white solid.

¹H NMR (d⁶ DMSO) 400 MHz δ6.44 (1H, s), 6.41 (1H, s), 3.76 (3H, s), 3.74 (3H, s), 2.21 (2H, s).

¹³C NMR (d⁶ DMSO) 100 MHz δ168.7, 160.4, 157.1, 136.3, 117.8, 106.6, 96.0, 55.7, 45.2, 19.3.

MS m/z (M+1) 197.0

### 2,4-Dimethoxy-6-methyl-benzoic acid ethyl ester (6)

Neat thionyl chloride (11.0 mL, 13.7 mmol, 1.0 eq) added drop wise to a cooled solution of 2,4-dimethoxy-6-methyl-benzoic acid (5) (1.7 g, 8.67 mmol, 1.0 eq) in DCM (˜20 mL) under nitrogen at 0° C. before allowing the mixture to warm to room temperature and stir for 18 h. The solvent was then removed in vacuo and the excess thionyl chloride removed by co-evaporation with anhydrous ethanol. The compound was purified by column chromatography using DCM as solvent to afford 1.7 g (88%) of 2,4-dimethoxy-6-methyl-benzoic acid ethyl ester (6) as light yellow oil.

¹H NMR (CDCl₃) 400 MHz δ6.31 (2H, s), 4.36 (2H, q), 3.80 (6H, s), 2.29 (3H, s), 1.36 (3H, t).

### 6,8-Dimethoxy-3-methyl-isochroman-1-one (ALM-7)

To a cooled solution of 2,4-dimethoxy-6-methyl-benzoic acid ethyl ester (6) (0.62 g, 2.76 mmol, 1.0 eq) in anhydrous THF (30 mL) at −78° C. under nitrogen was added dropwise a solution of LDA (2.0 ml of a 1.5M solution, 3.04 mmol, 1.1 eq) over 15 min. The mixture was stirred at −78° C. for a further 15 min before the solution was transferred by cannula and added dropwise to a stirred solution of acetalaehyde (0.62 ml, 18.0 mmol, 6.0 eq) in dry THF (15 ml) at −78° C. and then the reaction was left to warm up to room temperature over a period of ˜2 h. Ethanol (˜2 ml) was added and the solution concentrated in vacuo to leave a yellow oil. Purification by flash chromatography on silica using hexane/EtOAc afforded 0.15 g (25%) of 6,8-dimethoxy-3-methyl-isochroman-1-one (ALM-7) as a yellow solid.

¹H NMR (CDCl₃) 400 MHz δ6.41 (1H, s), 6.31 (1H., s), 4.51 (1H, m), 3.92 (3H, s), 3.86 (3H, s), 2.81 (2H, m), 1.47 (3H, d).

MS m/z (M+1)=223.2.

### 8-Hydroxy-6-methoxy-3-methylisochroman-1-one (ALM-8)

Solid AlCl₃ (2 granules, excess) was added to a stirred solution of 6,8-dimethoxy-3-methyl-isochroman-1-one (ALM-7) (20 mg, 0.09 mmol) in anhydrous 1,4-dioxane (˜3 ml) under nitrogen at room temperature before heating at reflux for 15 h. After cooling to room temperature water ˜(3 ml) was added and the solution extracted with ether. The combined organic extracts were dried, filtered and concentrated in vacuo. Purification by flash chromatography on silica using hexane/EtOAc afforded 18 mg (96%) of 8-hydroxy-6-methoxy-3-methylisochroman-1-one (ALM-8) as a brown solid.

¹H NMR (CDCl₃) 400 MHz δ6.30 (1H, s), 6.18 (1H, s), 4.60 (1H, m), 3.76 (3H, s), 2.80 (2H, m), 1.44 (3H, d).

MS m/z (M+1)=209.

### Preparation of Compounds ALM-9 to ALM-14

### Racemic 4-tert-butyldimethylsiloxypent-1-yne (7)

To a solution of 4-pentyn-2-ol (20.0 g, 0.24 mol, 1 eq) in anhydrous DMF (200 ml) at room temperature was added imidazole (32.5 g, 0.48 mol, 2 eq) and tert-butylchlorodimethylsilane (39.4 g, 0.26 mol, 1.08 eq) and the resulting pale yellow solution stirred for ˜17 h at room temperature. The mixture was diluted with water and extracted with ether. The combined organic extracts were washed with brine, dried over MgSO₄, and concentrated in vacuo to give 51.1 g (˜95%) of (+/−)-4-tert-butyldimethylsiloxypent-1-yne (7) as a colourless oil.

¹H-NMR CDCl₃ 300 MHz δ3.96 (1H, m), 2.36 (1H, ddd), 2.24 (1H, ddd′), 1.98 (1H, t), 1.23 (3H, d), 0.89 (9H, s), 0.08 (3H, s), 0.07 (3H, s).

¹³C-NMR (CDCl₃) 75 MHz δ81.9, 69.7, 67.5, 29.4, 25.8, 23.2, 18.1, −4.7, −4.8.

### Methyl (−/+)-5-tert-butyldimethylsiloxyhex-2-ynoate (8)

To a stirred solution of (+/−)-4-tert-butyldimethylsiloxypent-1-yne (7) (50.0 g, 0.24 mmol) in anhydrous THF (750 ml) at −78° C., was slowly added a solution of n-butylithium in hexane (1.6M, 178 ml, 0.29 mmol). The mixture was stirred for 30 min before the dropwise addition of methyl chloroformate (26.98 g, 0.29 mmol). The reaction was warmed to ambient temperature over 2.5 h. The mixture was diluted with water and extracted with ether (3×). The combined ether extracts were washed with brine, dried over MgSO₄ and concentrated in vacuo. The crude product was purified by column chromatography on silica using hexane/ether to afford 48.95 g (80%) of methyl (−/+)-5-tert-butyldimethylsiloxyhex-2-ynoate (8) as a colourless oil.

¹H-NMR (CDCl₃) 300 MHz δ4.02 (1H, m), 3.75 (3H, s), 2.49 (1H, dd), 2.38 (1H, dd), 1.24 (3H, d), 0.88 (9H, s), 0.08 (3H, s), 0.07 (3H, s).

¹³C-NMR (CDCl₃) 75 MHz δ154.1, 87.1, 74.1, 66.8, 52.5, 29.6, 25.7, 23.6, 18.0, −4.7, −4.9.

### Methyl (+/−)-2-(2-tert-butyldimethylsiloxypropyl)-6-methoxybenzoate (9)

Methyl (−/+)-5-tert-butyldimethylsiloxyhex-2-ynoate (8) (8.4 g, 32.76 mmol) was placed in a sealed tube along with dichloromaleic anhydride (20 mg), N-phenyl-β-naphthylamine (160 mg), and 1-methoxy-1,3-cyclohexadiene (8 ml, 4.38=mol). The tube was sealed and heated to 195° C. with stirring for 26 h. The heat was switched off and the reaction mixture cooled to room temperature and stirred overnight. The resulting crude brown oil was purified by column chromatography on silica using hexane/EtOAc to afford 5.15 g (46%) of methyl (+/−)-2-(2-tert-butyldimethylsiloxypropyl)-6-methoxybenzoate (9) as a yellow oil.

¹H-NMR (CDCl₃) 300 MHz δ7.26 (1H, m), 6.85 (1H, d), 6.78 (1H, d), 4.00 (1H, m), 3.90 (3H, s), 3.81 (3H, s), 2.71 (1H, dd), 2.62 (1H, dd), 1.12 (3H, d), 0.83 (9H, s), −0.08 and −0.19 (each 3H, s).

¹³C-NMR (CDCl₃) 75 MHz δ168.8, 156.4, 137.9, 129.8, 124.1, 123.6 and 108.9, 69.2, 56.0, 52.1, 43.7, 25.8, 23.9, 18.0, −5.0 and −5.1.

### Racemic Mellein Methyl Ether (ALM-12)

To a solution of methyl (+/−)-2-(2-tert-butyldimethylsiloxypropyl)-6-methoxybenzoate (9)<5.15 g, 15.2 mmol, 1.0 eq) in DCM (100 ml), was charged p-toluene sulfonic acid (0.29 g, 1.5 mmol) and the reaction stirred at room temperature for 25 h. The mixture was diluted with water, the aqueous layer extracted with DCM. The combined organic extracts were washed with brine, dried over MgSO₄ and concentrated in vacuo to afford a brown oil. Purification by flash chromatography on silica using diethyl ether afforded 2.66 g (92%) of racemic mellein methyl ether (ALM-12).

¹H-NMR (CDCl₃) 300 MHz δ7.45 (1H, m), 6.92 (1H, d), 6.80 (1H, d), 4.55 (1H, m), 3.95 (3H, s), 2.87 (2H, m), 1.48 (3H, d).

¹³C-NMR (CDCl₃) 75 MHz δ162.7, 161.2, 141.9, 134.4, 119.2, 113.7, 110.9, 74.1, 56.2, 36.1, 20.8.

### Racemic Mellein (ALM-9)

Racemic mellein methyl ether (ALM-12) (2.21 g, 115 mmol, 1.0 eq) was dissolved in 45% hydrobromic acid in acetic acid (102 ml, 0.57 mol) and the solution refluxed for 4 h. The solution was cooled to room temperature, diluted with water and neutralized with solid NaHCO₃. The resultant aqueous suspension was extracted with DCM. The combined organic extracts were washed with sat. NaHCO₃ brine, dried over MgSO₄ and concentrated in vacuo to afford a dark orange oil. Purification by flash chromatography on silica using hexane/EtOAc to afford a viscous oil which crystallized upon standing. This solid was recrystallised from hexane:ether (80:20) to afford 1.6 g (78%) of racemic mellein (ALM-9) as a white solid.

¹H-NMR (CDCl₃) 300 MHz δ11.03 (1H, s), 7.41 (1H, m), 6.39 (1H, d), 6.69 (1H, d), 4.73 (1H, m), 2.93 (2H, d), 1.53 (3H, d).

¹³C-NMR (CDCl₃) 75 MHz 6169.9, 162.2, 139.4, 136.1, 117.9,

### (3S)-mellein (ALM-10)

Prepared in same way as racemic mellein (ALM-9) from (S)-4-pentyn-2-ol.

¹H-NMR (CDCl₃) 300 MHz δ11.03 (1H, s), 7.41 (1H, m), 6.89 (1H, d), 6.69 (1H, d), 4.74 (1H, m), 2.94 (2H, d), 1.54 (3H, d).

¹³C-NMR (CDCl₃) 75 MHz δ170.0, 162.2, 139.4, 136.1, 117.9, 116.2, 108.3, 76.1, 34.6, 20.8.

### (3R)-mellein (ALM-11)

Prepared in same way as racemic mellein (ALM-9) from (R)-4-pentyn-2-ol.

¹H NMR (CDCl₃) 300 MHz δ11.03 (1H, s) 7.41 (1H, m), 6.89 (1H, d), 6.70 (1H, d), 4.74 (1H, m), 2.94 (2H, d), 1.54 (3H, d).

¹³C NMR (CDCl₃) 75 MHz δ169.9, 162.2, 139.4, 136.2, 117.9, 116.2, 108.3, 76.1, 34.6, 20.8.

(3S)-methoxy mellein (ALM-13)

Prepared in same way as racemic mellein (ALM-9) from (S)-4-pentyn-2-ol.

¹H NMR (CDCl₃) 400 MHz δ7.45 (1H, m), 6.92 (1H, d), 6.80 (1H, d), 4.56 (1H, m), 3.95 (3H, s), 2.87 (2H, m), 1.49 (3H, d).

¹³C NMR (CDCl₃) 100 MHz δ162.7, 161.2, 141.9, 134.4, 119.2, 113.7, 110.9, 74.8, 56.2, 36.1, 20.7.

(3R)-methoxy mellein (ALM-14)

Prepared in same way as racemic mellein (ALM-9) from (R)-4-pentyn-2-ol.

¹H NMR (CDCl₃) 400 MHz δ7.45 (1H, m), 6.92 (1H, d), 6.80 (1H, d), 4.56 (1H, m), 3.95 (3H, s), 2.87 (2H, m), 1.48 (3H, d).

¹³C NMR (CDCl₃) 100 MHz δ162.7, 161.2, 141.9, 134.4, 119.2, 113.7, 110.9, 74.1, 56.2, 36.1, 20.7.

Acetyl rac-Mellein (ALM-15)

Neat acetic anhydride (0.53 ml, 5.6 mmol, 2.0 eq) was aided to a stirred solution of racemic mellein (ALM-9) (550 mg, 2.82 mmol, 1.0 eq) and DMAP (69 mg, 0.56 mmol, 0.2 eq) in HPLC grade DCM (˜15 ml) under nitrogen at room temperature. The mixture was stirred until TLC indicated the reaction was complete (˜18 h) at which point water (˜10 ml) was then added and the mixture stirred for a further 30 min. The reaction mixture was then separated before washing the organic layer with saturated NaHCO₃ solution, brine, dried over MgSO₄, filtered and evaporated to dryness in vacuo to afford 578 mg (93%) of racemic acetyl mellein (ALM-15) as a pale yellow solid.

¹H NMR (CDCl₃) 400 MHz δ7.52 (1H, m), 7.13 (1H, m), 7.04 (1H, m), 4.66-4.60 (1H, m), 2.94 (2H, m), 2.36 (3H, s), 1.48 (3H, d).

¹³C NMR (CDCl₃) 100 MHz δ169.7, 162.1, 151.8, 141.3, 134.3, 125.2, 122.9, 117.8, 74.6, 35.4, 21.1, 20.6.

MS m/z (M+1) 220.9.

Racemic Mellein Ethyl Carbonate (ALM-16)

Neat ethyl chloroformate (128 mg, 0.112 ml, 1.12 mmol, 2.1 eq) was added to a stirred solution of racemic mellein (ALM-9) (100 mg, 0.56 mmol, 1.0 eq), DMAP (14 mg, 0.11 mmol, 0.2 eq) and triethylamine (130 mg, 0.18 ml, 1.28 mmol, 2.2 eq) in HPLC grade CHCl₃ (˜10 ml) under nitrogen at ˜0° C. The mixture was allowed to warm to ambient temperature and stirred until TLC indicated the reaction was complete (˜18 h). The reaction mixture was then washed the with saturated NaHCO₃ solution, 2M HCl, brine, dried over MgSO₄, filtered and evaporated to dryness in vacuo. The crude mixture was then purified by chromatography on silica using hexanes/EtOAc to afford 110 mg (80%) of racemic mellein ethyl carbonate (ALM-16) as a pale yellow solid.

¹H NMR (CDCl₃) 400 MHz δ7.45 (1H, m), 7.09-7.03 (2H, m), 4.60-4.52 (1H, m), 4.29-4.22 (1H, m), 2.90-2.81 (2H, m), 1.40 (3H, d), 1.32 (3H, t).

¹³C NMR (CDCl₃) 100 MHz δ162.1, 153.1, 151.9, 141.4, 134.4, 125.5, 122.3, 117.8, 74.7, 65.5, 35.2, 20.6, 14.2.

MS m/z (M+1)=250.9.

### General Procedure B

### For the Synthesis of Fatty Acid Esters of Racemic Mellein (Compounds ALM-17 to ALM-21)

To a solution of the acid (100 mg, 1 eq.) in DCM under nitrogen was added DCC (1 eq), DMAP (1 eq) and the reaction mixture cooled to 0° C. Racemic mellein (ALM-9) (1 eq.) was added and the reaction mixture stirred for 2 hrs at 0° C. The reaction mixture was filtered and then concentrated in vacuo. The residue was purified by column chromatography using hexane/ether. The product was dissolved in diethyl ether and washed 2M NaOH. The organic phase was separated, dried over MgSO₄ and the solvent concentrated in vacuo to afford the fatty acid esters of racemic mellein.

### Racemic Mellein Oleyl Ester (ALM-17)

Prepared following general procedure B. Obtained 50 mg (40%).

¹H NMR (CDCl₃) 400 MHz δ7.51 (1H, t), 7.12 (1H, d), 7.03 (1H, d), 5.36 (2H, m), 4.63 (1H, m), 2.93 (2H, m), 2.64 (2H, t), 2.01 (4H, d, br), 1.77 (2H, m), 1.48 (3H, d), 1.44-1.27 (20H, m), 0.88 (3H, t).

¹³C NMR (CDCl₃) 100 MHz δ172.4, 162.1, 152.0, 141.2, 134.2, 130.0, 129.8, 125.0, 122.9, 117.9, 74.6 35.4, 34.3 31.9, 29.8, 29.7, 29.5, 29.3, 29.2, 29.1, 27.2, 27.2, 24.44, 22.69, 20.63, 14.1.

### Racemic Mellein Linoleyl Ester (ALM-18)

Prepared following general procedure B. Obtained 102 mg (65%).

¹H NMR (CDCl₃) 400 MHz δ7.51 (1H, t), 7.11 (1H, d), 7.02 (1H, d), 5.36 (4H, m), 4.63 (1H, m), 2.93 (2H, d), 2.78 (2H, m), 2.64 (2H, t), 2.06 (4H, d, br), 1.77 (2H, m), 1.47 (3H, d), 1.43-1.24 (14H, m), 0.89 (3H, t).

¹³C NMR (CDCl₃) 100 MHz δ172.4 162.1 151.9, 141.2, 134.2 130.2 130.1, 130.0, 128.0, 127.9, 125.0, 122.9, 118.0, 100.0, 74.6, 35.9, 35.4, 34.2, 32.8, 31.5, 30.9, 29.6, 29.4 29.2, 29.1, 29.1, 27.2, 27.2, 26.4, 25.6, 25.5, 25.4, 25.4 24.7, 24.4, 22.6, 20.6, 14.1.

### Racemic Mellein α-Linolenyl Ester (ALM-19)

Prepared following general procedure B. Obtained 88 mg (56%).

¹H NMR (CDCl₃) 400 MHz δ7.51 (1H, t), 7.11 (1H, d), 7.02 (1H, d), 5.34 (6H, m), 4.63 (1H, m), 2.93 (2H, d), 2.81 (4H, t), 2.65 (2H, t), 2.07 (4H, m), 1.77 (2H, m), 1.48 (3H, d), 1.44-1.31 (8H, m), 0.97 (3H, t).

¹³C NMR (CDCl₃) 100 MHz δ172.4, 162.1, 151.9, 141.2, 134.2, 131.9, 130.3, 128.3, 128.3, 127.7, 127.1, 125.0, 122.9, 119.9, 74.6, 35.4, 34.2, 29.6, 29.2, 29.1, 29.1, 27.2, 25.6, 25.5, 25.4, 24.4, 20.6, 14.3.

### Racemic Mellein γ-Linolenyl Ester (ALM-20)

Prepared following general procedure B. Obtained 79 mg (50%).

¹H NMR (CDCl₃) 400 MHz δ7.51 (1H, t), 7.12 (1H, d), 7.02 (1H, d), 5.38 (6H, m), 4.63 (1H, m), 2.93 (2H, d), 2.82 (3H, m, br), 2.67 (2H, t), 2.14 (2H, q), 2.06 (2H, q), 1.80 (2H, m), 1.54-1.25 (12H, m), 0.88 (3H, t).

¹³C NMR (CDCl₃) 100 MHz δ172.2, 162.1, 151.9, 141.3, 134.2, 130.4, 129.7, 128.4, 128.2, 128.2, 127.6, 125.1, 122.9, 117.9, 74.6, 35.4, 34.1, 31.5, 29.3, 29.3, 29.1, 27.2, 27.0, 25.9, 22.6, 20.6, 14.1.

### Racemic Mellein Heneicosanoyl Ester (ALM-21)

Prepared following general procedure B. Obtained 52 mg (35%).

¹H NMR (CDCl₃) 400 MHz δ7.44 (1H, t), 7.05 (1H, d), 6.98 (1H, d), 4.56 (1H, m), 2.86 (2H, d), 2.57 (2H, t), 1.70 (2H, m), 1.41 (3H, d), 1.36-1.17 (34H, m), 0.81 (3H, t).

### Racemic Ortho-Fluoromellein (ALM-22) and Racemic Para-Fluoromellein (ALM-23)

F-TEDA(1-Chloromethyl-4-fluoro-1,4-diazoniabicyclo[2.2.2]octane bis(tetrafluoroborate), Selectfluor®) (1.10 g, 3.08 mmol, 1.1 eq) was added in one portion to a stirred solution of racemic mellein (ALM-9) (500 mg, 2.80 mmol, 1.0 eq) in methanol (˜15 ml) at room temperature under nitrogen before heating to reflux for 3 days. After cooling the solution was diluted with DCM (5 ml) and the precipitate removed by filtration. The filtrate was evaporated to dryness in vacuo and then purified by flash chromatography on silica using EtOAc/hexanes.

The following compounds were then isolated in order of elution:

140 mg (26%) of raceanic para-fluoromellein (ALM-23) as a white powder;

¹H NMR (CDCl₃) 400 MHz δ10.75 (1H, s), 7.22 (1H, m), 6.89-6.85 (1H, m), 4.77-4.69 (1H, m), 3.16 (1H, dd), 2.75 (1H, dd), 1.57 (3H, d).

¹³C NMR (CDCl₃) 100 MHz 6169.2, (169.2), 158.4, 152.5, 150.1, 124.6, (124.4), 123.7, (123.4), 116.9, (116.8, 108.1, (108.1) 76.0, 27.7, 20.8.

MS m/z (M+1) 197.1.

This was followed by 68 mg of recovered mellein and finally racemic ortho fluoromellein (ALM-22) 52 mg (17%) as a white solid.

¹H NMR (CDCl₃) 400 MHz 610.9 (1H, s), 7.20-7.14 (1H, m) 76.59-6.56 (1H, m), 4.71-4.63 (1H, m), 2.89-2.78 (1H, m), 1.46 (3H, d).

¹³C NMR (CDCl₃) 100 MHz δ169.5, 151.35, 150.1, 148.9, 134.2, 122.0, 117.0, 110.0, 34.1, 20.6.

MS m/z (M−1) 195.4.

### Racemic 4-Bromomellein (ALM-24)

Bromine (89 mg, 0.028 ml, 0.56 mmol, 1.0 eq) was aided dropwise to a stirred solution of racemic mellein (ALM-9) (100 mg, 0.56 mmol, 1.0 eq) in HPLC grade DCM under nitrogen at ˜0° C. The reaction mixture was allowed to slowly warm to ambient and stirred for ˜18 h. The reaction mixture was washed with ˜10 wt % sodium thiosulfate solution and then water (2×) before being dried over MgSO₄ and evaporated to dryness in vacuo to afford a white solid. The crude solid was purified by flash chromatography on silica using hexanes/EtOAc to afford in order of elution: 105 mg (73%) of racemic 4-bromomellein (ALM-24) as a white platey solid:

¹H NMR (CDCl₃) 400 MHz δ7.61 (1H, d), 6.84 (1H, d), 4.76-4.67 (1H, m), 3.20 (1H, dd), 2.81 (1H, dd), 1.57 (3H, d).

¹³C NMR (CDCl₃) 100 MHz δ169.4, 161.7, 139.5, 138.4, 118.1, 111.1, 109.8, 75.6, 34.9, 20.8.

MS m/z (M+1)=257.1 and 259.0.

### Racemic Ortho-Iodomellein (ALM-25) and Racemic Para-Iodomellein (ALM-26)

To a stirred suspension of NaHCO₃ (471 mg, 5.61 mol, 2.0 eq), AgOCOCF₃ (682 mg, 3.08 mmol, 1.1 eq) and racemic mellein (ALM-9) (500 mg, 2.80 mmol, 1.0 eq) in HPLC grade chloroform (25 ml) under nitrogen at room temperature was added dropwise a solution of iodine (712 mg, 2.80 mmol, 1.0 eq) dissolved in chloroform (25 ml) over a period of ˜1 h. The reaction mixture was stirred for a further 1 h during which time the reaction was allowed to reach ambient temperature. The reaction was filtered through celite and evaporated to dryness in vacuo to afford a yellow solid. The crude solid was purified by flash chromatography on silica using hexanes/EtOAc to isolate, in order of elution:

283 mg (33%) of the para isomer (ALM-26) as a white solid,

¹H NMR (CDCl₃) 400 MHz δ11.25 (1H, s), 7.83 (1H, d), 6.73 (1H, d), 4.75-4.66 (1H, m), 3.10-3.05 (1H, m), 2.85-2.78 (1H, m), 1.57 (3H, d).

¹³C NMR (CDCl₃) 100 MHz δ169.6, 162.6, 145.5, 142.0, 118.8, 110.0, 85.0, 75.6, 40.0, 20.7.

MS m/z (M−1)=303.3.

then recovered racemic mellein 45 mg, and finally 470 mg (55%) of the ortho isomer (ALM-25) also as a white solid.

¹H NMR (CDCl₃) 400 MHz δ11.9 (1H, s), 7.8 (1H, d), 6.5 (1H, d), 4.8-4.7 (1H, m), 2.96-2.90 (2H, m), 1.56 (3H, d).

¹³C NMR (CDCl₃) 100 MHz δ169.3, 160.9, 145.3, 139.7, 119.7, 108.5, 82.9, 76.2, 34.3, 20.7.

MS m/z (M−1)=303.2.

### Racemic 2,4-di-iodomellein (ALM-27)

To a stirred suspension of NaHCO₃ (78 mg, 0.93 mmol, 2.0 eq), AgOCOCF₃ (113 mg, 0.51 mmol, 1.1 eq) and racemic mellein (ALM-9) (102 mg, 0.46 mmol, 1.0 eq) in HPLC grade chloroform (5 ml) under nitrogen at room temperature was added solid iodine (176 mg, 0.70 mmol, 1.5 eq) in one portion. The reaction mixture was stirred for 2.5 h. The reaction mixture was filtered through celite and the filtrate solution washed with ˜10 wt % thiosulfate solution and then brine before being dried over MgSO₄ and evaporated to dryness in vacuo to afford a yellow oil. The crude oil was purified by flash chromatography on silica using hexanes/EtOAc to afford racemic 2,4-di-iodomellein 11 mg (29%) as a white solid (ALM-27) and 83 mg of recovered acyl mellein.

¹H NMR (CDCl₃) 400 MHz δ8.33 (1H, s), 4.75-4.67 (1H, m), 3.06 (1H, dd), 2.80 (1H, dd), 1.57 (3H, d).

¹³C NMR (CDCl₃) 100 MHz δ169.1, 161.5, 153.5, 142.3, 109.6, 85.8, 85.0, 75.7, 39.8, 20.7.

MS m/z (M+1)=431.0.

### Racemic 4-iodomethoxymellein (ALM-28)

To a stirred suspension of NaHCO₃ (86 mg, 1.02 mmol, 2.0 eq), AgOCOCF₃ (142 mg, 0.51 mmol, 1.1 eq) and racemic mellein (ALM-9) (98 mg, 0.51 mmol, 1.0 eq) in HPLC grade chloroform (8 ml) under nitrogen at room temperature was added solid iodine (169 mg, 0.56 mmol, 1.5 eq) in one portion. The reaction mixture was stirred for 2 h. The reaction mixture was filtered through celite and the filtrate evaporated to dryness in vacuo to afford a pale yellow oil. The crude oil was purified by flash chromatography on silica using hexanes/EtOAc to afford 140 mg (87%) of racemic 4-Iodomethoxymellein (ALM-28) as white solid.

¹H NMR (CDCl₃) 400 MHz δ7.82 (1H, d), 6.67 (1H, d), 4.47-4.41 (1H, m), 3.86 (3H, s), 2.95 (1H, dd), 2.71 (1H, dd), 1.44 (3H, d).

¹³C NMR (CDCl₃) 100 MHz δ162.0, 161.4, 144.3, 144.0, 115.8, 113.3, 86.9, 73.5, 56.4, 41.5, 21.1.

MS m/z (M+1)=319.1

### Racemic ortho-acetylmellein (ALM-29)

Solid racemic acetyl mellein (ALM-15) (200 mg, 0.96 mmol, 1.0 eq) was admixed with powdered AlCl₃ (412 mg, 3.09 mmol, 3.2 eq) and heated in an oil bath at 120° C. for 20 min and then to 165° C. for 3 h. Upon cooling the reaction was cautiously quenched, solubilised with ice/water, and filtered. The filtrate was acidified with 2M HCl and the precipitate collected. Purification by chromatography using hexane/EtOAc afforded 14 mg (7%) of racemic ortho-acetyl mellein (ALM-29) as a white solid.

¹H NMR (CDCl₃) 400 MHz δ12.35 (1H, s), 8.01 (1H, d), 6.77 (1H, d), 4.77-4.71 (1H, m), 2.99-2.97 (2H, m), 2.70 (3H, s), 1.56 (3H, d).

¹³C NMR (CDCl₃) 100 MHz δ198.2, 162.6, 145.1, 137.3, 125.4, 117.9, 109.6, 75.8, 35.0, 31.5, 20.7.

MS m/z (M+1)=221.1.

### Racemic 2,4-dinitromellein (ALM-30)

Concentrated (65%) HNO₃ (0.1 ml, 1.47 mmol, 1.05 eq) was added to a chilled (˜0° C.) solution of neat concentrated (95-98%) H₂SO₄ (˜3 ml) and this mixture was stirred for ˜10 min before the addition of solid racemic mellein (ALM-9) (250 mg, 1.40 mmol, 1.0 eq) in one portion. A small volume of DCM (˜3 ml) was then added to aid solubility of mellein. The biphasic mixture was then stirred for a further 1.5 h during which time the temperature was allowed to reach ambient. The reaction was quenched by pouring onto a 10 wt % solution of Na₂SO₄ in water (10 ml). After further dilution with water (10 ml) the solution was extracted with CH₂Cl₂. The combined extracts were dried over Na₂SO₄ and then evaporated to dryness in vacuo to afford 281 mg (75%) of racemic 2,4-dinitromellein (ALM-30) as a solid yellow powder.

¹H NMR (CDCl₃) 400 MHz δ13.45 (1H, s), 8.99 (1H, s), 4.82-4.75 (1H, m), 3.74 (1H, dd), 3.29 (1H, dd), 1.66 (3H, d).

¹³C NMR (CDCl₃) 100 MHz δ168.4, 160.0, 141.6, 137.3, 136.4, 128.8, 111.7, 75.7, 32.2, 20.5.

MS m/z (M−1)=267.4.

### Scheme 7

### Preparation of 8-hydroxy-3-methyl-1-oxo-isochroman-7-carboxylic acid (ALM-33) from racemic ortho-iodomellein (ALM-25)

### Racemic O-acetyoxy-2-iodomellein (10)

To a stirred solution of racemic 2-iodomellein (ALM-25) (100 mg, 0.33 mmol, 1.0 eq) and DMAP (8 mg) in HPLC grade DCM (˜20 ml) under nitrogen at room temperature was added neat acetic anhydride (0.12 ml, 1.2 mmol, 4.0 eq) and the reaction monitored by TLC. After 48 h the reaction mixture was washed with saturated NaHCO₃ solution (2×) then brine, dried over MgSO₄ and evaporated to dry-ness in vacuo to afford 102 mg (89%) of racemic O-acetyoxy-2-iodomellein (10) as a white solid.

¹H NMR (CDCl₃) 400 MHz δ7.96 (1H, d), 6.91 (1H, d), 4.63 (1H, s, br), 2.92-2.90 (2H, m), 2.42 (3H, s), 1.52 (3H, d).

MS m/z (M+1)=346.7

### Racemic O-acetyl-8-hydroxy-3-methyl-1-oxo-isochroman-7-carboxylic acid methyl ester (11)

A suspension of racemic O-acetyoxy2-iodomellein (10) (101 mg, 0.29 mmol, 1.0 eq) sodium acetate (79 mg, 0.58 mmol, 2.0 eq) and palladium acetate (˜13 mg) in methanol (100 ml) was subjected to an atmosphere of carbon monoxide at 5 bar and a temperature of 40° C. for 24 h. The reaction mixture was filtered through celite before being evaporated to dryness in vacuo. The crude oil was then purified by chromatography on silica using hexane/EtOAc to afford 28 mg (68%) of racemic O-acetyl-8-hydroxy-3-methyl-1-oxo-isochroman-7-carboxylic acid methyl ester (11) as a white solid together with 48 mg of recovered starting material.

¹H NMR (CDCl₃) 400 MHz δ8.06 (1H, d), 7.13 (1H, d), 4.58-4.53 (1H, m), 3.82 (3H, s), 2.92-2.90 (2H, m), 2.33 (3H, s), 1.47 (3H, d).

¹³C NMR (CDCl₃) 100 MHz δ169.3, 164.1, 161.0, 145.7, 136.1, 124.9, 124.9, 124.4, 119.3, 74.3, 52.5, 35.7, 20.9, 20.7.

### Racemic O-methoxy-2-iodomellin (12)

To a stirred suspension of racemic 2-iodomellein (ALM-25) (100 mg, 0.33 mmol, 1.0 eq) and K₂CO₃ (309 mg, 2.24 mmol, 7.0 eq) in acetone (˜15 ml) under nitrogen at room temperature was added neat dimethylsulfate (0.15 ml, 1.6 mmol, 5.0 eq) and the reaction heated to reflux and was then monitored by TLC. After ˜1 h the reaction mixture was cooled to room temperature before quenching with water. The mixture was extracted with CHCl₃ (3×), the extracts then combined, dried over MgSO₄ and evaporated to dryness in vacuo to afford 102 mg (˜98%) of crude racemic O-methoxy 2-iodomellein (12) as a yellow oil.

¹H NMR (CDCl₃) 400 MHz δ7.92 (1H, d), 6.79 (1H, d), 4.60-4.55 (1H, m), 3.94 (3H, s), 2.89-2.87 (2H, m), 1.49 (3H, d).

¹³C NMR (CDCl₃) 100 MHz δ161.4, 161.3, 143.9, 142.0, 124.6, 119.1, 92.7, 74.3, 62.2, 35.6, 20.6.

### Racemic O-methoxy-8-hydroxy-3-methyl-1-oxo-isochroman-7-carboxylic acid methyl ester (13)

A suspension of crude racemic O-methoxy2-iodomellein (12) (102 mg, 0.32 mmol, 1.0 eq) sodium acetate (899 mg, 0.65 mmol, 2.0 eq) and palladium acetate (˜15 mg) in methanol (100 ml) was subjected to an atmosphere of carbon monoxide at 5 bar and a temperature of ±0° C. for 24 h. The reaction mixture was filtered through celite before being evaporated to dryness in vacuo. The crude oil was teen purified by chromatography on silica using hexane/EtOAc to afford 14 mg (54%) of racemic O-methoxy-8-hydroxy-3-methyl-1-oxo-isochroman-7-carboxylic acid methyl ester (13) as a white solid together with 70 mg of recovered starting material.

¹H NMR (CDCl₃) 400 MHz δ7.90 (1H, d), 7.03 (1H, d), 4.60-4.55 (1H, ma), 3.99 (3H, s), 3.93 (3H, s), 2.99-2.89 (2H, m), 1.57 (3H, d).

¹³C NMR (CDCl₃) 100 MHz δ166.0, 162.1, 161.5, 145.6, 135.8, 126.1, 122.4, 119.6, 74.0, 63.6, 52.5, 36.2, 20.6.

MS m/z (M+1)=251.0.

### Scheme 8

### Preparation of 8-hydroxy-3-methyl-1-oxo-isochroman-7-carboxylic acid methyl ester (ALM-34) via alkyne aldehyde (14)

Alkyne aldehyde (14)

To a stirred solution of 4-tert-butyldimethylsiloxypent-1-yne (7) (1.0 g, 5.0 mmol, 1.0 eq) in dry THF (˜10 ml) under nitrogen at −60° C. was added drop wise nBuLi (3.9 ml of 1.36M, 1.05 eq). After 5 min neat anhydrous DMF (0.78 ml, 10 mmol, 2.0 eq) was added and the mixture stirred at −60° C. for a further 5 min. The cold bath was removed and the mixture allowed to warm up to) room temperature over ˜30 min. The reaction mixture was then poured onto a stirred biphasic mixture of aqueous K₂HPO₄ (˜25 ml) and ether (˜25 ml) at 0° C. The organic layer was separated, and washed with water (2×). The water washes were combined and back extracted with ether. The ether extracts were combined, dried over MgSO₄ and evaporated to dryness to afford 1.1 g (98%) of crude alkyne aldehyde (14) as a yellow oil, which was used directly in the next step.

¹H NMR (CDCl₃) 400 MHz δ9.10 (1H, s, br), 3.99-3.94 (1H, m), 2.49-2.37 (2H, m), 1.18 (3H, d), 0.81 (9H, s), 0.01 (3H, s), 0.00 (3H, s).

¹³C NMR (CDCl₃) 100 MHz δ176.9, 96.5, 82.7, 66.7, 29.6, 25.7, 23.3, −3.0, −2.9.

### Diester (15)

To a stirred cooled solution of dimethyl 1,3-acetonedicarboxylate (0.78 g, 4.47 mmol, 1.0 eq) in anhydrous THF (˜8 ml) at 0° C. under nitrogen was added solid NaH (128 mg, 5.36 mmol, 1.2 eq) in one portion. After ˜10 min a solution of alkyne aldehyde (14) (1.02 g, 5.36 mmol, 1.2 eq) in dry THF (˜2 ml) was added drop wise over 10 min. The mixture was allowed to slowly warm up to room temperature over ˜2 h and then stirred for a further 15 h. The reaction mixture was then poured onto dilute 2M HCl and separated retaining the organic layer. The aqueous layer was extracted with EtOAc and all the organic extracts combined washed with brine, dried over MgSO₄ and evaporated to dryness. The residual oil was purified by chromatography on silica using hexanes/EtOAc to afford 365 mg (19%) of the diester intermediate (15) as an orange oil.

¹H NMR (CDCl₃) 400 MHz δ11.2 (1H, s), 7.83 (1H, d), 6.87 (1H, d), 4.12-4.08 (1H, m), 4.00 (6H, s), 2.80-2.78 (2H, m), 1.21 (3H, d), 0.89 (9H, s), 0.00 (3H, s), −0.14 (3H, s).

¹³C NMR (CDCl₃) 100 MHz δ170.2, 167.7, 158.7, 158.7, 145.6, 130.2, 123.5, 122.3, 110.7, 69.0, 52.4, 52.3, 44.2, 40.9, 25.6, 23.8, 18.0, −5.0.

MS m/z (M+1)=383.0.

### 8-hydroxy-3-methyl-1-oxo-isochroman-7-carboxylic acid methyl ester (ALM-34)

To a stirred solution of intermediate (15) (291 mg, 0.76 mmol, 1.0 eq) in dry DCM (˜5 ml) under nitrogen at room temperature was added para-toluenesulfonic acid hydrate (˜15 mg) and the mixture monitored by TLC. After ˜48 h the mixture was washed with saturated NaHCO₃ solution, dried over MgSO₄ and evaporated to dryness in vacuo to give a white solid. Purification by chromatography on silica using hexanes/EtOAc afforded 170 mg (95%) of 8-hydroxy-3-methyl-1-oxo-isochroman-7-carboxylic acid methyl ester (ALM-34) as a white powder.

### Scheme 9

### Preparation of 8-hydroxy-3-methyl-1-oxo-isochroman-7-carboxylic acid (ALM-33) and 8-hydroxy-3-methyl-1-oxo-isochroman-7-carboxylic acid methyl ester (ALM-34)

### Dimethyl 2-hydroxy-4-methylbenzene-1,3-dicarboxylate (17)

To a suspension of sodium methoxide (26.0, 0.48 mol, 1.12 eq) in anhydrous THF (250 mL) under nitrogen at 0° C. was added dropwise a solution of ethyl formate (31.89 g, 0.43 mol, 1.0 eq) and acetone (25.00 g, 0.43 mol, 1.0 eq), maintaining the temperature <5° C. The reaction was stirred at 0° C. for 15 min then warmed to ambient temperature at which it was then stirred for 15 min. The reaction was evaporated to dryness, to afford crude sodium formyl acetone (16), which was dissolved in methanol (500 mL) under nitrogen. Dimethyl 1,3-acetonedicarboxylate (71.21 g, 0.41 moles, 0.95 eq) was added dropwise, maintaining the temperature <25° C. The reaction was stirred for 16 h at room temperature. The reaction was concentrated to dryness, the residue was diluted with 2M HCl, and extracted into ethyl acetate. The combined organic extracts were washed with saturated brine, dried over MgSO₄, filtered and concentrated in vacuo. Purification by vacuum distillation (˜0.1 mbar, b.p. ˜1,5-120° C.) to afford 21.76 g (24%) of dimethyl 2-hydroxy-4-methylbenzene-1,3-dicarboxylate (17) as a pale yellow oil which solidified upon standing.

¹H NMR (CDCl₃) 400 MHz δ7.74 (1H, d), 6.72 (1H, d), 3.94 (3H, s), 3.91 (3H, s), 2.33 (3H, s).

### 8-Hydroxy-3-methyl-1-oxo-isochroman-7-carboxylic acid methyl ester (ALM-34)

To a solution of LDA (54.5 mL, 1.8M solution, 98.1 mmol, 2.2 eq) in anhydrous THF (180 mL) under nitrogen cooled to −78° C. was added dropwise a solution of dimethyl 2-hydroxy-4-methylbenzene-1,3-dicarboxylate (17) (10.00 g, 44.6 mmol, 1 eq) in anhydrous THF (20 mL) at −78° C. After stirring for 20 min, acetaldehyde (6.12 g, 139.0 mmol, 3.1 eq) was added dropwise at −78° C. The reaction was stirred at −78° C. for 30 min then warmed t<) 0° C., then stirred for 45 min. The reaction was quenched at 0° C. by the addition of acetic acid (5.15 mL), and then warmed to ambient temperature. The reaction was diluted with 2M HCl and extracted with EtOAc. The combined organic extracts were washed with brine, dried over MgSO₄, filtered and concentrated in vacuo. The residue was recrystallised from hexane/EtOAc to afford 5.12 g (49%) of 8-hydroxy-3-methyl-1-oxo-isochroman-7-carboxylic acid methyl ester (ALM-34) as a beige solid.

¹H NMR (CDCl₃) 400 MHz δ8.04 (1H, d), 6.76 (1H, d), 4.72 (1H, m), 3.94 (3H, s), 2.97 (2H, d), 1.54 (3H, d).

MS m/z (M−1)=235.

### 8-Hydroxy-3-methyl-1-oxo-isochroman-7-carboxylic acid (ALM-33)

To a suspension of 8-hydroxy-3-methyl-1-oxo-isochroman-7-carboxylic acid methyl ester (ALM-34) (4.75 g, 20.1 mmol, 1 eq) in ethanol (75 mL) was added 10% sodium hydroxide ^{w}/ᵥ aqueous solution (15 mL, 37.5 mmol, 1.9 eq). The mixture refluxed for 1 h and then cooled to ambient temperature. The reaction mixture was concentrated in vacuo. The residue was diluted with water and washed with EtOAc and the organic layer discarded. The aqueous layer was adjusted to pH 1 with 2M HCl, saturated with NaCl, and extracted with ethyl acetate. The combined organic extracts were dried over MgSO₄, filtered and concentrated in vacuo. The solid was slurried in methanol, to afford 3.46 g (77%) of 8-hydroxy-3-methyl-1-oxo-isochroman-7-carboxylic acid (ALM-33) as an off-white solid.

¹H NMR (CDCl₃) 400 MHz δ7.97 (1H, d), 6.89 (1H, d), 4.72 (1H, m), 3.00 (2H, m), 1.41 (3H, d).

¹³C NMR (CDCl₃) 100 MHz δ138.8, 165.4, 161.8, 146.7, 136.5, 117.8, 115.7, 111.8, 74.8, 34.4, 20.2.

MS m/l (M+1)=223.

### Preparation of 8-hydroxy-1-oxo-isochroman-7-carboxylic acid methyl ester (ALM-68)

To a solution of LDA (10.9 mL, 1.8M solution, 19.6 mmol, 2.2 eq) in anhydrous THF (35 mL) under nitrogen cooled to −78° C. was added dropwise a solution of dimethyl 2-hydroxy-4-methylbenzene-1,3-dicarboxylate (17) (2.00 g, 8.92 mmol, 1 eq) in anhydrous THF (5 mL) at −78° C. After stirring for 20 min, paraformaldehyde (0.83 g, 27.7 mmol, 3.1 eq) was added dropwise at −78° C. The reaction was stirred at −78° C. for 30 min then warmed to 0° C., and then stirred for 45 min. The reaction was quenched at 0° C. by the addition of acetic acid (1.05 mL), and them warmed to ambient temperature. The reaction was diluted with 2 M HCl and extracted into ethyl acetate. The combined organic extracts were washed with brine, dried over MgSO₄, filtered and concentrated in vacuo. The residue was purified by column chromatography using hexane/EtOAc to afford 468 mg (24%) of 8-hydroxy-1-oxo-isochroman-7-carboxylic acid methyl ester (ALM-68) as a beige solid.

¹H NMR (CDCl₃) 400 MHz δ8.05 (1H, d), 6.78 (1H, d), 4.57 (2H, t), 3.95 (3H, s), 3.08 (2H, t).

### Preparation of 8-hydroxy-3,3-dimethyl-1-oxo-isochroman-7-carboxylic acid methyl ester (ALM-71)

To a solution of LDA (10.9 mL, 1.8M solution, 19.6 mmol, 2.2 eq) in anhydrous THF (35 mL) under nitrogen cooled to −78° C. was added dropwise a solution of dimethyl 2-hydroxy-4-methylbenzene-1,3-dicarboxylate (17) (2.00 g, 8.92 mmol, 1 eq) in anhydrous THF (5 mL) at −78° C. after stirring for 20 min, acetone (1.61 g, 27.7 mmol, 3.1 eq) was added dropwise at −78° C. The reaction was stirred at −78° C. for 30 min then warmed to 0° C., and then stirred for 45 min. The reaction was quenched at 0° C. by the addition of acetic acid (1.05 mL), and then warmed to ambient temperature. The reaction was diluted with 2M HCl and extracted into EtOAc. The combined organic extracts were washed with brine, dried over MgSO₄, filtered and concentrated in vacuo. The residue was purified by column chromatography using DCM/hexane/EtOAc to afford 1.03 g (46%) of 8-hydroxy-3,3-dimethyl-1-oxo-isochroman-7-carboxylic acid methyl ester (ALM-71) as a beige solid.

¹H NMR (CDCl₃) 400 MHz δ8.05 (1H, d), 6.75 (1H, d), 3.94 (3H, s), 3.03 (2H, s), 1.49 (6H, s).

MS m/z (M+1)=251.

### Preparation of 8-hydroxy-3,3-dimethyl-1-oxo-isochroman-7-carboxylic acid (ALM-72)

To a suspension of 8-hydroxy-3,3-dimethyl-1-oxo-isochroman-7-carboxylic acid methyl ester (ALM-71) (900 mg, 3.6 mmol) in ethanol (13.5 mL) was added 10% W/v sodium hydroxide aqueous solution (2.7 mL). The mixture refluxed for 1 h, cooled to ambient temperature and concentrated in vacuo. The residue was diluted with water and washed with ethyl acetate. The organic layer was discarded. The aqueous layer was adjusted to pH 1 with 2M HCl, saturated and extracted with EtOAc. The combined organic extracts were dried over MgSO₄, filtered and concentrated in vacuo. The solid was slurried in methanol, to afford 849 mg (100%) of 8-hydroxy-3,3-dimethyl-1-oxo-isochroman-7-carboxylic acid (ALM-72) as an off-white solid.

¹H NMR (CD₃OD) 400 MHz δ8.00 (1H, d), 6.78 (1H, d), 3.02 (2H, s), 1.35 (6H, s).

### General Procedure C

### For the Preparation of Esters of 8-hydroxy-3-methyl-1-oxo-isochroman-7-carboxylic acid (compounds ALM-35 to ALM-43)

To the reaction vessel were charged 8-hydroxy-3-methyl-1-oxo-isochroman-7-carboxylic acid (ALM-33) (250 mg, 1.13 mmol, 1.0 eq), the alcohol (1.0 eq), DMAP (151 mg, 1.24 mmol, 1.1 eq) and anhydrous DCM (10 mL) under nitrogen. To this was added DCC (255 mg, 1.24 mmol, 1.1 eq) and the reaction mixture stirred at room temperature overnight. The mixture was filtered through celite and the filtrate concentrated in vacuo. The crude material was purified by column chromatography using hexane/EtOAc to afford pure 8-hydroxy-3-methyl-1-oxo-isochroman-7-carboxylic acid ester.

### L-3-Phenyl Lactic Acid Ester (ALM-35)

Coupling with benzyl protected L-3-phenyl lactic acid following general procedure C. Obtained 660 mg (63%). Debenzylated (Pd/C, H₂, EtOAc) to afford ALM-35. Obtained 93 mg (18%).

1H NMR (CDCl₃) 400 MHz δ8.01 (1H, d), 7.34-7.21 (5H, m), 6.75 (1H, d), 5.46 (1H, m), 4.71 (1H, m), 3.30 (2H, m), 2.96 (2H, d), 1.53 (3H, d).

¹³C NMR (CDCl₃) 100 MHz δ171.4, 168.3, 164.8, 162.7, 145.6, 138.2, 136.2, 129.4, 128.4, 126.9, 117.7, 316.1, 109.9, 75.6, 73.6, 37.3, 35.0, 20.5.

MS m/z (M+1)=371.

### Benzyl Ester (ALM-36)

Prepared following general procedure C. Obtained 116 mg (33%).

¹H NMR (CDCl₃) 400 MHz δ8.05 (1H, d), 7.48-7.31 (5H, m), 6.72 (1H, d), 5.38 (2H, s), 4.71 (1H, m), 2.95 (2H, d), 1.53 (3H, d).

¹³C NMR (CDCl₃) 100 MHz δ168.2, 165.6, 162.8, 145.3, 138.0, 135.8, 128.6, 128.3, 128.2, 117.5, 117.0, 110.1, 75.5, 66.9, 35.1, 20.6.

MS m/z (M+1)=313.

### Phenyl Ester (ALM-37)

Prepared following general procedure C. Obtained 90 mg (27%).

¹H NMR (CDCl₃) 400 MHz δ8.15 (1H, d), 7.35 (2H, d), 7.18 (3H, m), 6.75 (1H, d), 4.68 (1H, m), 2.93 (2H, d), 1.48 (3H, d).

¹³C NMR (CDCl₃) 100 MHz δ168.5, 163.9, 163.1, 150.6, 145.7, 138.5, 129.5, 126.0, 121.7, 117.7, 116.6, 110.1, 75.6, 35.1, 20.7.

MS m/z (M+1)=299.

### Cyclohexyl Ester (ALM-38)

Prepared following general procedure C. Obtained 220 mg (64%).

¹H NMR (CDCl₃) 400 MHz δ8.01 (1H, d), 6.74 (1H, d), 5.07 (1H, m), 4.69 (1H, m), 2.96 (2H, d), 1.94 (2H, m, br), 1.80 (2H, m, br), 1.65-1.30 (9H, m).

¹³C NMR (CDCl₃) 10 MHz δ165.9, 162.8, 145.2, 137.3, 117.4, 110.5, 75.2, 73.7, 35.3, 31.53, 25.4, 23.6, 20.6.

MS m/z (M+1)=305.

### Tert-Butyl Ester (ALM-39)

Prepared following general procedure C. Obtained 132 mg (40%).

¹H NMR (CDCl₃) 400 MHz δ7.95 (1H, d), 6.71 (1H, d), 4.68 (1H, m), 2.94 (2H, d), 1.62 (9H, s), 1.52 (3H, d).

¹³C NMR (CDCl₃) 100 MHz δ167.0, 165.9, 162.8, 145.1, 137.2, 117.9, 117.2, 110.6, 100.0, 82.5, 75.1, 35.4, 28.2, 20.6.

MS m/z (M−1)=377.

### Hexyl Ester (ALM-40)

Prepared following general procedure C. Obtained 100 mg (41%).

¹H NMR (CDCl₃) 400 MHz δ8.02 (1H, d), 6.75 (1H, d), 4.71 (1H, m), 4.34 (2H, t), 2.96 (2H, d), 1.77 (2H, q), 1.53 (3H, d), 1.44 (2H, m), 1.34 (4H, m), 0.89 (3H, t).

¹³C NMR (CDCl₃) 100 MHz δ167.7, 166.2, 162.7, 145.2, 137.6, 117.4, 117.2, 110.3, 75.3, 65.6, 35.2, 31.4, 28.6, 25.6, 22.5, 20.7, 14.0.

MS m/z (M+1)=307.

### Dodecyl Ester (ALM-41)

Prepared following general procedure C. Obtained 300 mg (68%)

¹H NMR (CDCl₃) 400 MHz δ8.02 (1H, d), 6.74 (1H, d), 4.71 (1H, m), 4.33 (2H, t), 2.96 (2H, d), 1.77 (2H, m), 1.53 (3H, d), 1.44 (1H, m, br), 1.32 (17H, m, br), 0.88 (3H, t).

¹³C NMR (CDCl₃) 100 MHz δ167.7, 166.2, 162.7, 145.2, 137.6, 117.4, 117.1, 110.3, 75.3, 65.6, 35.2, 31.9, 29.6, 29.6, 29.6, 29.5, 29.3, 29.3, 28.6, 25.9, 22.7, 20.6, 14.1.

MS m/z (M+1)=391.

### Oleyl Ester (ALM-42)

Prepared following general procedure C. Obtained 319 mg (60%).

¹H NMR (CDCl₃) 400 MHz δ8.02 (1H, d), 6.74 (1H, d), 5.34 (2H, m), 4.71 (1H, m), 4.33 (2H, t), 2.96 (2H, d), 2.02 (4H, d, br), 1.76 (2H, m), 1.53 (3H, d), 1.47-1.22 (22H, m), 0.88 (3H, t).

MS m/z (M+1)=473.

### γ-Linolenyl Ester (ALM-43)

Prepared following general procedure C. Obtained 70 mg (40%).

¹H NMR (CDCl₃) 400 MHz δ8.02 (1H, d), 6.74 (1H, d), 5.36 (6H, m), 4.71 (1H, m), 4.34 (2H, t), 2.96 (2H, d), 2.81 (4H, broad m), 2.08 (4H, m), 1.76 (2H, m), 1.55 (3H, d), 1.49-1.25 (10H, m), 0.88 (3H, t).

### General Procedure D

### For the Preparation of Amides of 8-hydroxy-3-methyl-1-oxo-isochroman-7-carboxylic acid (compounds ALM-44 to ALM-52)

To a stirred solution of 8-hydroxy-3-methyl-1-oxo-isochroman-7-carboxylic acid (ALM-33) (250 mg, 1.12 mmol, 1.0 eq) in dry DMF (10 ml) under nitrogen at room temperature was added solid N,N-carbonyl diimidazole (192 mg, 1.18 mmol, 1.05 eq) and the reaction monitored by TLC. Once the reaction was judged complete (˜1 h) the amine (1.0 eq) was added and the reaction stirred further until TLC indicated that the reaction was complete. The crude mixture was then evaporated to dryness in vacuo. The residual oil was taken up into EtOAc or CHCl₃, washed with water, dried over MgSO₄ and evaporated to dryness to afford the amide of 8-hydroxy-3-methyl-1-oxo-isochroman-7-carboxylic acid. All subsequent couplings were conducted on an identical scale unless otherwise stated.

### Benzyl Amide (ALM-44)

Prepared following general procedure D. Obtained 221 mg (63%) as a yellow solid.

¹H NMR (CDCl₃) 400 MHz δ12.7-12.6 (1H, s, br), 8.33 (2H, m), 7.29-7.11 (5H, m), 6.75 (1H, d), 4.72-4.58 (1H, m), 4.59 (2H, d), 2.90-2.86 (2H, m), 1.45 (3H, d).

¹³C NMR (CDCl₃) 100 MHz δ170.4, 164.0, 160.3, 143.6, 139.1, 138.4, 128.7, 127.8, 127.4, 127.1, 119.7, 118.6, 108.7, 76.4, 43.9, 34.6, 20.7.

MS m/z (M+1)=312.1.

### Aniline Amide (ALM-45)

Prepared following general procedure D. Obtained 62 mg (19%) as a white solid.

¹H NMR (CDCl₃) 400 MHz δ13.0 (1H, s), 9.91 (1H, s), 8.38 (1H, d), 7.63-7.60 (2H, dd), 7.31-7.26 (2H, dd), 7.08-7.04 (1H, dd), 6.80 (1H, d), 4.75-4.67 (1H, m), 2.95-2.85 (2H, m), 1.48-1.44 (3H, d).

¹³C NMR (CDCl₃) 100 MHz δ(unassigned mix of rotamers) 170.6, 162.0, 160.0, 143.8, 139.1, 138.1, 129.0, 124.4, 120.6, 120.0, 119.9, 118.9, 108.8, 76.5, 34.6, 20.7.

MS m/z (M+1)=298.0.

Together with CDI derivative (ALM-46)

Prepared following general procedure D. Obtained 198 mg (64%) as a white solid

¹H NMR (CDCl₃) 400 MHz δ8.94 (1H, s), 8.21 (1H, d), 7.36 (2H, s), 6.69 (1H, d), 4.72-4.65 (1H, m), 2.97-2.88 (2H, m), 1.55 (3H, d).

### Pyrrolidine Amide (ALM-47)

Prepared following general procedure D. Obtained 250 mg (80%) as a yellow oil.

¹H NMR (CDCl₃) 400 MHz δ11.45 (1H, s), 7.49 (1H, d), 6.76 (1H, d), 4.79-4.71 (1H, m), 3.66 (2H, t), 3.36 (2H, t), 2.99-2.94 (2H, m), 2.04-1.86 (4H, m), 1.54 (3H, d).

¹³C NMR (CDCl₃) 100 MHz δ169.7, 166.0, 158.0, 140.7, 134.9, 126.1, 118.1, 108.6, 76.2, 47.5, 45.8, 34.5, 25.9, 24.5, 20.7.

MS m/z (M+1)=276.5.

### Methyl Amide (ALM-48)

Prepared following general procedure D. Obtained by taking residual solid up into chloroform and stirring with amberlite IR-120+ resin, filtered off resin, dried over MgSO₄ and evaporated to dryness to afford 161 mg (61%) of the amide as a white solid.

¹H NMR (CDCl₃) 400 MHz δ12.7-12.6 (1H, s, br), 8.31 (1H, d), 7.96 (1H, s, br), 6.76 (1H, d), 4.75-4.66 (1H, m), 2.95-2.79 (5H, m), 1.48 (3H, d).

¹³C NMR (CDCl₃) 100 MHz δ170.5, 164.6, 160.1, 143.1, 138.9, 119.8, 118.5, 108.6, 76.4, 34.6, 26.5, 20.6.

MS m/z (M+1)=236.1.

### Hexyl Amide (ALM-49)

Prepared following general procedure D. Obtained 120 mg (35%) as a yellow solid.

¹H-NMR (CDCl₃) 400 MHz 6127-12.6 (1H, s, br), 8.32 (1H, d), 8.00 (1H, s, br), 6.76 (1H, d), 4.74-4.66 (1H, m), 3.41-3.36 (2H, m), 2.96-2.90 (2H, m), 1.58-1.51 (2H, m), 1.48 (3H, d), 1.47-1.30 (6H, m), 0.86-0.75 (3H, m).

¹³C NMR (CDCl₃) 100 MHz δ170.5, 163.8, 160.2, 143.0, 139.0, 120.0, 118.5, 108.6, 76.3, 40.5, 34.6, 31.5, 29.4, 26.7, 22.5, 20.6, 14.0.

MS m/z (M+1)=306.2.

### N-Methyl-N-Hexyl Amide (ALM-50)

Prepared following general procedure D. On 200 mg scale ALM-50 obtained 150 mg (52%) as a colourless oil as a 1:1 mixture of unassigned rotamers.

¹H NMR (CDCl₃) 400 MHz δ11.37 (0.5H, s), 11.35 (0.5H, s), 7.45 (0.5H, d), 7.41 (0.5H, d), 6.75 (1H, d), 4.77-4.71 (1H, m), 3.55 (1H, m), 3.18 (1H, m), 3.09 (1.5H, s), 2.96-2.94 (2H, m), 2.90 (1.5H, s), 1.70-1.63 (1H, m), 1.63-1.51 (3H, d), 1.40-1.10 (7H, m), 0.94-0.81 (3H, m).

¹³C NMR (CDCl₃) 100 MHz δ169.7, 169.7, 167.6, 167.4, 157.9, 140.5, 140.4, 134.9, 134.7, 125.6, 125.3, 118.1, 118.0, 108.5, 108.4, 76.2, 50.8, 47.3, 36.0, 34.5, 32.4, 31.6, 31.3, 28.0, 26.9, 26.4, 26.0, 22.6, 22.4, 20.7, 14.0, 13.9.

MS m/z (M+1)=320.3

### Dodecyl Amide (ALM-51)

Prepared following general procedure D. Obtained 298 mg (63%) as a yellow wary solid.

¹H NMR (CDCl₃) 400 MHz δ8.42 (1H, d), 8.07 (1H, s, br), 6.85 (1H, d), 4.78 (1H, m), 3.47 (2H, q), 2.99 (2H, m), 2.73 (1H, m, br), 1.64-1.55 (8H, m, br), 1.26 (L4H, m, br), 0.88 (3H, t).

¹³C NMR (CDCl₃) 100 MHz δ170.5, 163.9, 160.2, 143.0, 139.0, 120.1, 118.4, 108.6, 76.4, 39.9, 34.6, 31.9, 29.6, 29.6, 29.5, 29.3, 27.1, 22.7, 20.6, 14.1.

MS m/z (M+1)=390.

### Oleyl Amide (ALM-52)

Prepared following general procedure D. Obtained 172 mg (32%).

¹H NMR (CDCl₃) 400 MHz δ8.42 (1H, d), 8.07 (1H, s, br), 6.85 (1H, d), 5.35 (2H, m), 4.78 (1H, m), 4.78 (1H, m), 3.46 (2H, q), 2.99 (2H, m), 2.01 (4H, m), 1.60 (8H, m), 1.33 (19H, m), 0.86 (3H, t).

MS m/z (M+1)=472.

### Compounds ALM-53 to ALM-67

### General Procedure E

### For the Coupling of t-Butyl Protected Amino Acids with 8-hydroxy-3-methyl-1-oxo-isochroman-7-carboxylic acid

To a stirred, cooled solution of 8-hydroxy-3-methyl-1-oxo-isochroman-7-carboxylic acid (ALM-33) (500 mg, 2.25 mmol, 1.0 eq) and HOBt (320 mg, 2.36 mmol, 1.05 eq) in dry DMF (˜20 ml) under nitrogen at 0° C. was added solid EDC.HCl (453 mg, 2.36 mmol, 1.05 eq) followed by neat DIPEA (0.43 ml, 2.47 mmol, 1.1 eq) and the mixture stirred at 0° C. for ˜1 h before allowing to warm to room temperature. The amino acid.HCl (1.0 eq) was then added in one portion followed by DIPEA (0.43 ml, 2.47 mmol, 1.1 eq) and the mixture stirred at room temperature for 18 h. The crude mixture was evaporated to dryness in vacuo before taking up into CHCl₃. The organic layer was washed sequentially with: saturated NaHCO₃; 10% wt aqueous citric acid solution; saturated NaHCO₃; and then water, dried over MgSO₄, filtered and evaporated to dryness. Purification by chromatography using hexanes/EtOAc afforded the pure t-butyl protected amino acid amide of 8-hydroxy-3-methyl-1-oxo-isochroman-7-carboxylic acid.

### General Procedure F

### For the Deprotection of t-butyl Protected Amino Acid Amide of 8-hydroxy-3-methyl-1-oxo-isochroman-7-carboxylic acid

Neat TFA (5 ml, excess) was added to a stirred solution of t-butyl protected amino acid amide of 8-hydroxy-3-methyl-1-oxo-isochroman-7-carboxylic acid (˜1.7 mmol, 1 eq) in CHCl₃ (˜10 ml) and the mixture stirred for 15-18 h. Once complete conversion was achieved the mixture was evaporated to dryness in vacuo. The residual oil was taken back up into CHCl₃ and this solution was washed with water, dried over MgSO₄ filtered and evaporated to dryness to afford pure amino acid amide of 8-hydroxy-3-methyl-1-oxo-isochroman-7-carboxylic acid.

### L-Phenyl Alanine Tert-Butyl Ester Amide (ALM-53)

Prepared following general procedure E. Obtained 710 mg (74%) as a yellow oil.

¹H NMR (CDCl₃) 400 MHz δ8.52 (1H, s), 8.30 (1H, s) 7.17 (5H, s), 6.76 (1H, s), 4.90 (1H, m), 4.70 (1H, m), 3.13 (2H, d), 2.91 (2H, m), 1.47 (3H, d), 1.33 (9H, s).

¹³C NMR (CDCl₃) 100 MHz δ170.6, 170.3, 163.3, 143.4, 138.9, 136.5, 129.6, 128.3, 126.9, 119.5, 118.4, 108.7, 82.2, 76.3, 54.5, 38.3, 34.7, 27.9, 20.7.

MS m/z (M−1)=424.

### L-phenyl Alanine Amide (ALM-54)—Racemic Ochratoxin B

Prepared following general procedure F.

¹H NMR (CDCl₃) 400 MHz δ12.75-12.70 (1H, s, br), 8.53-8.51 (1H, m), 8.35 (1H, d), 7.33-7.23 (5H, m), 6.84 (1H, d), 5.00-4.95 (1H, m), 4.81-4.72 (1H, m), 3.37 (1H, dd), 3.21 (1H, dd), 3.02-2.97 (2H, m), 1.56 (3H, d).

¹³C NMR (d⁶ DMSO) 100 MHz δ(as a 1:1 mixture of diastereoisomers) 172.5, 172.5, 169.1, 169.0, 163.7, 163.7, 159.5, 159.5, 144.8, 137.0, 136.8, 136.8, 129.2, 128.3, 126.6, 118.5, 118.5, 118.3, 109.4, 76.1, 53.8, 36.6, 33.6, 20.1.

MS m/z (M−1) 368.4.

### Leucine Tert-Butyl Ester Amide (ALM-55)

Prepared following general procedure E. On a 300 mg scale isolated 370 mg (70%) of ALM-55 after chromatography as a yellow oil.

¹H NMR (CDCl₃) 400 MHz δ12.80-12-75 (1H, d), 8.47 (1H, s, br), 8.39 (1H, d), 6.84 (1H, d), 4.80-4.73 (2H, m), 3.00-2.99 (2H, m), 2.04-1.65 (3H, m), 1.49 (9H, s), 0.96 (6H, d).

¹³C NMR (CDCl₃) 100 MHz δ(as a mixture of unassigned rotamers) 172.1, 170.4, 170.3, 163.5, 160.4, 143.4, 143.4, 139.0, 119.6, 118.4, 108.7, 81.7, 81.7, 76.3, 76.3, 52.0, 41.9, 41.8, 34.6, 34.6, 28.0, 25.1, 23.9, 22.9, 22.2, 20.8.

MS m/z (M+1)=392.0.

### Leucine Amide (ALM-56)

Prepared following general procedure F. Obtained 256 mg (81%) as a brown oil.

¹H NMR (CDCl₃) 400 MHz δ12.2 (1H, d), 9.49 (1H, s, br), 8.52-8.50 (1H, m), 8.37 (1H, d), 6.86 (1H, d), 4.82-4.74 (2H, m), 3.06-2.94 (2H, m), 1.88-1.72 (3H, m), 1.54 (3H, d), 0.99-0.88 (6H, m).

¹³C NMR (CDCl₃) 100 MHz δ(as a mixture of unassigned rotamers) 176.8, 176.8, 170.4, 170.3, 164.6, 164.4, 160.5, 143.9, 143.8, 139.1, 118.9, 118.7, 108.7, 76.4, 51.6, 40.9, 34.6, 34.6, 25.0, 22.9, 21.9, 20.6.

MS m/z (M+1)=336.1.

### Proline Tert-Butyl Ester Amide (ALM-57)

Prepared following general procedure E. Obtained 190 mg (37%) as a yellow solid after chromatography.

¹H NMR (CDCl₃) 400 MHz δ(as a mixture of unassigned rotamers) 12.40 (1H, s, br), 12.35 (1H, s, br), 7.46 (1H, d), 7.39 (1H, m), 6.68 (1H, d), 6.65 (1H, m), 4.68-4.64 (1H, m), 4.49-4.45 (1H, m), 4.30-4.20 (1H, m), 3.73-3.70 (1H, m), 3.47-3.39 (2H, m), 2.89-2.87 (2H, m), 2.23-2.19 (1H, m), 1.98-1.89 (3H, m), 1.47 (3H, d), 1.43 (9H, s), 1.22 (9H, s).

¹³C NMR (CDCl₃) 100 MHz δ(as a mixture of unassigned rotamers) 171.4, 171.1, 169.7, 169.6, 166.4, 166.1, 158.2, 158.2, 157.6, 157.4, 141.0, 140.8, 136.0, 135.9, 135.3, 135.3, 129.0, 128.2, 125.4, 125.3, 11:9.0, 118.0, 108.6, 108.6, 81.5, 81.3, 76.3, 76.2, 60.6, 59.8, 59.7, 47.9, 47.9, 46.5, 46.5, 34.5, 34.4, 31.3, 31.3, 29.7, 29.6, 28.0, 27.8, 24.7, 23.9, 22.8, 20.7, 20.6.

MS m/z (M+1)=376.0.

### Proline Amide (ALM-58)

Prepared following general procedure F. Obtained 190 mg (37%) as a brown oil.

¹H NMR (CDCl₃) 400 MHz δ(as a mixture of unassigned rotamers) 12.45 (1H, d), 12.35 (1H, d), 8.49 (2H, s, br), 7.48-7.45 (1H, m), 7.36-7.33 (1H, m), 6.70 (1H, d), 6.66 (1H, d), 4.72-4.60 (2H, m), 4.35-4.29 (2H, m), 3.71-3.67 (1H, m), 3.51-3.35 (2H, m), 2.93-2.81 (2H, m), 2.27-2.12 (2H, m), 2.08-1.88 (2H, m), 1.50-1.40 (4H, m).

¹³C NMR (CDCl₃) 100 MHz δ(as a mixture of unassigned rotamers) 174.0, 173.9, 169.7, 169.6, 168.1, 158.0, 157.9, 141.7, 135.4, 135.3, 124.0, 124.0, 118.3, 118.3, 108.8, 108.7, 77.3, 76.8, 76.3, 76.3, 59.6, 59.6, 48.4, 48.4, 34.5, 34.5, 29.7, 28.7, 24.6, 20.7, 20.7.

MS m/z (M+1)=320.1.

### β-Alanine Tert-Butyl Ester Amide (ALM-59)

Prepared following general procedure E. Obtained 223 mg (47%).

¹H NMR (CDCl₃) 400 MHz δ8.44 (1H, s, br), 8.40 (1H, d), 6.84 (1H, d), 4.77 (1H, m), 3.72 (2H, q), 2.99 (2H, m), 2.57 (2H, t), 1.56 (3H, d), 1.47 (9H, s).

MS m/z (M+1)=350.

### β-Alanine Amide (ALM-60)

Prepared following general procedure F. Obtained 162 mg (98%).

¹H NMR (CDCl₃) 400 MHz δ8.64 (1H, s, br), 8.37 (1H, d), 6.85 (1H, d), 4.78 (1H, m), 3.78 (2H, q), 3.00 (2H, m), 2.74 (2H, t), 1.56 (3H, d).

¹³C NMR (CDCl₃) 100 MHz δ176.8, 170.3, 164.7, 160.7, 143.7, 138.9, 119.2, 118.6, 108.7, 76.3, 35.2, 34.6, 34.0, 20.6.

MS m/z (M+1)=294.

### Glycine Tert-Butyl Ester Amide (ALM-61)

Prepared following general procedure E. Obtained 162 mg (36%).

¹H NMR (CDCl₃) 400 MHz δ8.58 (1H, s, br), 8.40 (1H, d), 6.85 (1H, d), 4.78 (1H, m), 4.19 (2H, d), 3.00 (2H, m), 1.56 (3H, d), 1.51 (9H, s).

MS m/z (M+1)=336.

### Glycine Amide (ALM-62)

Prepared following general procedure F. Obtained 109 mg (90%).

¹H NMR (DMSO-d₆) 400 MHz δ8.69 (1H, s, br), 8.15 (1H, d), 7.02 (1H, d), 4.88 (1H, m), 4.07 (1H, d), 3.17-3.01 (2H, m), 1.49 (3H, d).

¹³C NMR (CDCl₃) 100 MHz δ171.0, 168.8, 164.4, 159.7, 144.8, 136.7, 118.4, 118.4, 109.4, 76.0, 41.4, 33.7, 20.1.

MS m/z (M+1)=280.

### L-Phenyl Glycine Tert-Butyl Ester Amide (ALM-63)

Prepared following general procedure E. Obtained 340 mg (46%) as an off-white solid

¹H NMR (CDCl₃) 400 MHz δ12.80-12.70 (1H, d), 9.10-9.07 (1H, m), 8.26 (1H, d), 7.38 (2H, m), 7.29-7.21 (3H, m), 6.73 (1H, d), 5.62-5.60 (1H, m), 4.70-4.66 (1H, m), 2.91-2.88 (1H, m), 1.46 (3H, d), 1.32 (9H, s).

¹³C NMR (CDCl₃) 100 MHz δ168.4, 168.0, L 61.2, 158.7, 141.7, 137.1, 135.7, 135.6, 126.8, 126.2, 125.2, 117.5, 116.6, 106.8, 80.6, 80.6, 74.4, 55.9, 32.7, 25.9, 18.7.

MS m/z (M−1)=410.4.

### L-Phenyl Glycine Amide (ALM-64)

Prepared following general procedure F. Obtained 120 mg (98%) as a white powder.

¹H NMR (CDCl₃) 400 MHz δ13.85 (1H, d), 9.15-9.13 (1H, m), 8.34 (1H, d), 7.51 (2H, dd), 7.39-7.31 (3H, m), 6.82 (1H, d), 5.77 (1H, d), 4.80-4.73 (1H, m), 3.02-2.92 (2H, m), 1.55 (3H, d).

¹³C NMR (CDCl₃) 100 MHz δ(as a mixture of unassigned rotamers) 174.5, 170.3, 163.8, 160.6, 143.9, 139.1, 136.0, 136.0, 129.1, 128.7, 127.4, 113.9, 118.6, 108.8, 76.7, 76.3, 57.3, 34.6, 20.6.

MS m/z (M+1)=356.1.

### L-Aspartate Di-Tert-Butyl Ester Amide (ALM-65)

Prepared following general procedure E. Obtained 320 mg (53%).

¹H NMR (CDCl₃) 400 MHz δ8.93 (1H, broad d), 8.38 (1H, d), 6.84 (1H, d), 4.95 (1H, m), 4.78 (1H, m), 3.01-2.84 (4H, m), 1.55 (3H, d), 1.49 (9H, s), 1.45 (9H, s).

MS m/z (M+1)=450.

### L-Aspartate Amide (ALM-66)

Prepared following general procedure F. Obtained 254 mg (95%).

¹H NMR (CD₃OD) 400 MHz δ8.13 (1H, d), 6.86 (1H, d), 4.88 (1H, t), 4.54 (2H, m), 3.04-2.81 (4H, m), 1.42 (3H, d).

MS m/z (M−1)=336.

### L-Lysine Amide (ALM-67)

Prepared following general procedures E and F. Obtained 155 mg (95%).

¹H NMR (CD₃OD) 400 MHz δ8.21 (1H, d), 6.99 (1H, d), 4.72 (1H, m), 4.54 (2H, m), 3.17-2.93 (4H, m), 2.08 (1H, m), 1.93 (1H, m), 1.74 (2H, m), 1.55 (5H, m).

MS m/z (M+1)=351.

L-Phenyl Alanine Tert-Butyl Ester Amide of Compound ALM-68 (ALM-69)

Prepared following general procedure E with compound ALM-68. Obtained 193 mg (41%) as a yellow oil after chromatography.

¹H NMR (CDCl₃) 400 MHz δ12.5 (1H, s, br), 8.51 (1H, d), 8.30 (1H, d), 7.21-7.14 (5H, m), 6.78 (1, d), 4.92-4.87 (1H, m), 4.52 (2H, t), 3.13 (2H, m), 3.02 (2H, t), 1.33 (9H, s).

¹³C NMR (CDCl₃) 100 MHz δ169.5, 168.9, 162.2, 159.5, 142.8, 137.9, 135.4, 128.5, 127.3, 125.9, 118.4, 117.4, 108.0, 81.2, 67.0, 53.5, 37.2, 26.9, 26.6.

MS m/z (M+1)=412.0.

### L-Phenyl Alanine Amide of Compound ALM-68 (ALM-70)

Prepared following general procedure F. Obtained 155 mg (93%) as a yellow oil after chromatography.

¹H NMR (CDCl₃) 400 MHz δ12.6 (1H, s), 8.56 (1H, d), 8.35 (1H, d), 7.31-7.07 (5H, m), 6.86 (1H, d), 5.07-5.02 (1H, m), 4.59 (2H, t), 3.35 (1H, dd), 3.22 (1H, dd), 3.09 (2H, t),

¹³C NMR (CDCl₃) 100 MHz δ175.0, 170.0, 164.4, 160.6, 144.2, 139.0, 135.9, 129.4, 128.7, 127.2, 118.8, 118.5, 109.1, 68.1, 54.4, 37.4, 27.6.

MS m/z (M+1)=356.1.

### L-Phenyl Alanine Tert-Butyl Ester Amide of Compound ALM-71 (ALM-73)

Prepared following general procedure E with compound ALM-71. Obtained 185 mg (33%) as a yellow oil after chromatography.

¹H NMR (CDCl₃) 400 MHz δ12.75 (1H, s, br), 8.54 (1H, d), 8.30 (1H, d), 7.22-7.14 (5H, m), 6.75 (1H, d), 4.93-4.88 (1H, m), 3.14 (2H, m), 3.02 (1H, dd), 2.96 (2H, s), 1.43 (3H, s), 1.41 (3H, s), 1.34 (9H, s).

¹³C NMR (CDCl₃) 100 MHz δ170.6, 169.8, 163.4, 160.5, 142.7, 139.0, 136.5, 129.6, 128.4, 127.0, 119.5, 119.0, 108.3, 82.8, 82.2, 54.5, 39.3, 38.3, 38.0, 28.0, 27.4, 27.2.

MS m/z (M+1)=440.0.

### L-Phenyl Alanine Amide of Compound ALM-73 (ALM-74)

Prepared following general procedure F. Obtained 85 mg (50%) as a yellow solid.

¹H NMR (CDCl₃) 400 MHz δ12.75 (1H, s), 8.52 (1H, d), 8.27 (1H, d), 7.23-7.13 (5H, m), 6.74 (1H, d), 5.01-4.96 (1H, m), 3.27 (1H, dd), 3.14 (1H, dd), 2.95 (2H, s), 1.43 (3H, s), 1.41 (3H, s).

¹³C NMR (CDCl₃) 100 MHz δ175.2, 169.8, 164.4, 160.5, 143.1, 139.1, 135.9, 129.4, 128.7, 127.2, 119.2, 118.8, 108.3, 82.9, 54.3, 39.3, 37.5, 27.4, 27.2.

### Acetonide Compound (ALM-75)

To a solution of 8-hydroxy-3-methyl-1-oxo-isochroman-7-carboxylic acid (ALM-33) (200 mg, 0.9 mmol, 1.0 eq) in trifluoroacetic acid (5 ml) cooled to 0° C. was added dropwise trifluoroacetic anhydride (0.38 ml, 2.7 mmol, 3.0 eq) followed by acetone (0.33 ml, 4.5 mol, 5.0 eq). The reaction was allowed to warm to room temperature and stirred overnight. The reaction mixture was concentrated to half volume. The residue was dissolved in EtOAc (20 mL) and stirred with saturated NaHCO₃ solution for 30 mins. The organic layer was separated and the aqueous extracted with EtOAc. The organic layers were dried over MgSO₄, filtered and evaporated to dryness in vacuo to afford 209 mg (89%) of acetonide compound (ALM-75) as a beige solid.

¹H NMR (CDCl₃) 400 MHz δ 8.13 (1H, d), 7.00 (1H, d), 4.63 (1H, m), 2.97 (2H, m), 1.81 (3H, s), 1.79 (3H, s), 1.41 (3H, d).

¹³C NMR (CDCl₃) 100 MHz δ 160.55, 160.16, 157.36, 148.94, 134.39, 121.09, 114.38, 113.72, 107.29, 74.12, 36.29, 25.96, 25.83, 20.63.

MS m/z (M+1)=381.4.

### Biological Data Methods

The compounds of the present invention have been found to have anti-cancer activity as determined by the MTT assay cell counts and cell survival assays which may be determined by the following procedures.

### MTT Assay

The MTT assay was used initially to determine the anti-proliferative activity of the test compounds. In the MTT assay a yellow thiazolyl blue tetrazolium bromide salt (MTT) is reduced, in metabolically active cells, to form insoluble purple formazan crystals which are solubilised by the addition of dimethyl sulphoxide (DMSO). Absorbance readings can then be determined spectrophotometrically and a relationship established between control untreated cells and drug treated cells enabling the quantification of anti-proliferative changes as a result of drug treatment (Mosmann, 1983).

For the MTT assay 1×10³ cells (e.g. MCF-7, MDA-MB-468, SkMe128 or MalMe3M) per well (180 μl) were seeded in 96 well plates with 6 replicates for each treatment in the appropriate tissue culture medium e.g. RPMI 1640 containing 1% penicillin streptomycin and 10% foetal calf serum (Gibco) and allowed to attach overnight in a tissue culture incubator (37° C., 5% CO₂). Wells containing medium only were used as blanks. Following the attachment period, cells were treated with appropriate drug concentrations (20 μl added per well). 40 mg/ml stock drug solutions, in DMSO, were stored at −20° C. and on the day of drug treatment working solutions of 4 mg/ml were prepared in medium which was further diluted to achieve final drug dilution volumes of 20 μl. Initial screening was determined over a concentration range of 1-200 μg/ml. For potent compounds the assay was repeated over a narrower concentration range to determine accurate IC50 values. Cells were exposed to drug for 96 h continual exposure (37° C., 5% CO₂). Following the 96 h incubation, 50 μl MTT solution (1 g MTT/500 ml PBS) was added per well and the plates were further incubated for 4 h (37° C., 5% CO₂). After incubation the MTT and medium was removed from the wells and 100 μl DMSO added per well. Plates were agitated on an orbital mixer platform (Stuart Scientific) for 10 minutes. Absorbance was measured at 570 nm on a Biotrak II (Amersham Bioscience) plate reader. IC50 values were calculated comparing control and drug treated cells.

### Cell Counts

Cell counts were determined in 24 well plates (Nunc) in triplicate. Cells (MCF-7, MDA-MB-468, SkMel28 or MalMe3M) were seeded at 1×10³ cells per well in RPMI1640 medium containing 1% penicillin streptomycin and 10% foetal calf serum (Gibco) and allowed to attach overnight in a tissue culture incubator (37° C., 5% CO₂). The following day the medium was removed and replaced with 1 ml medium per well containing the appropriate drug concentrations to be tested. 40 mg/ml stock drug solutions in DMSO were stored at −20° C. and on the day of drug treatment working solutions of 4 mg/ml were prepared in medium from which the final drug dilutions were prepared. Cells were maintained in an incubator (37° C., 5% CO₂) for up to 7 days post treatment. Daily cell counts were performed following drug treatment. On the day of the cell counts the medium was removed from each well and 0.5 ml trypsin-EDTA solution added to each well. Cells were replaced into the incubator for 10 min to allow them to detach from the plastic plate. 250 μl of trypsinised cell solution was added to 10 mls of isoton and cells counted on a Coulter counter. Cell growth profiles were then compared for treated and control cells over a 7 day period.

### Cell Survival Assay

Cell survival following drug treatment was determined in 24 well plates (Nunc) in triplicate. Cells (MCF-7, MDA-MB-468, SkMel28 or MalMe3M) were seeded at 1×10³ cells per well in RPMI1640 medium containing 1% penicillin streptomycin and 10% foetal calf serum (Gibco) and allowed to attach overnight in a tissue culture incubator (37° C., 5% CO₂). The following day the medium was removed and replaced with 1 ml medium per well containing the appropriate drug concentrations to be tested. 40 mg/ml stock drug solutions in DMSO were stored at −20° C. and on the day of drug treatment working solutions of 4 mg/ml were prepared in medium from which the final drug dilutions were prepared. Cells were maintained in an incubator (37° C., 5% CO₂) for 7 days. The medium was then removed and 1 ml fresh medium added to each well. Cells were further incubated for 7 days (37° C., 5% CO₂). Medium was then removed and the cells were fixed for 5 min with 1 ml ice cold methanol per well. Cells were then stained with 1 ml 0.5% crystal violet solution (1 g crystal violet, 50 ml methanol, 150 ml distilled H₂0) per well for 5 min at room temperature on a shaker. The crystal violet solution was removed from each well and 1 ml distilled H₂0 added to each well for 5 min at room temperature on a shaker. The H₂0 was removed and a further wash in distilled H₂0 was performed to remove excess stain. Plates were allowed to dry at room temperature. Crystal violet was resorbed from the cells by adding 1 ml 0.1M sodium citrate (50 ml 0.2M Sodium Citrate, 50 ml ethanol) solution to each well. Plates were shaken at room temperature for 20 min. 200 μl of each sample was then transferred to a 96 well plate and the absorbance measured on a Biotrak II (Amersham Bioscience) plate reader at 570 nm. The background signal (sodium citrate) was subtracted from the crystal violet measurements and values were compared to control cells untreated with drug.

### Cell Cycle Analysis

Assessment of cell cycle analysis was performed using propidium iodide staining of cells and subsequent analysis on a Beckton Dickinson flow cytometer. Cells (MCF-7, MDA-MB-468, SkMel28 or MalMe3M) were seeded at 1×10³ cells/ml in T25 tissue culture flasks (Nunc) in RPMI1640 medium containing 1% penicillin streptomycin and 10% foetal calf serum (Gibco) and allowed to attach overnight in a tissue culture incubator (37° C., 5% CO₂). The following day the medium was removed and replaced with 5 ml medium per flask containing the appropriate drug concentrations to be tested. 40 mg/ml stock drug solutions in DMSO were stores at −20° C. and on the day off drug treatment working solutions of 4 mg/ml were prepared in medium from which the final drug dilutions were prepared. Cells were maintained in an incubator (37° C., 5% CO₂) for up to 96 h post treatment. After 24, 48, 72 or 96 h time points the medium from each flask was collected, stored on ice, and the remaining cells in the flasks were trypsinised by aiding 1 ml trypsin-EDTA solution to each flask. Cells were replaced into the incubator for 10 min to allow them to detach from the plastic plate. The cells in each flask were collected by adding the appropriate medium removed from each flask back to the corresponding flask and then placing medium containing both the attached and floating cells into a 15 ml tube. Cells were centrifuged at 4° C. for 5 min at 1500 rpm and the supernatant removed from each tube. The remaining cells were washed twice in ice cold PBS and then fixed in ice cold methanol overnight at −20° C. On the day of analysis of cells each tube was centrifuged at 4° C. for 5 min at 1500 rpm and the methanol removed and the cells washed twice in ice cold PBS. 20 μl propidium iodide solution (1 mg/ml) and 20 μl RNase A (10 mg/ml solution) were added to 1 ml PBS per tube and the cells were incubated for 30 min at 37° C. Cells were analysed on the flow cytometer and cell cycle changes were assessed.

### Western Blotting

Western blotting was used to assess drug induced changes of cellular proteins. Cells were seeded in P90 tissue culture vessels and treated with drugs at a range of concentrations for 72 and 96 h. Following incubation of cells for the required duration the medium was collected from the cells and attached cells were scraped from the tissue culture vessel and collected into the medium. Excess medium was removed by centrifugation at 4° C. for 5 minutes at 150 rpm and the supernatant removed from each tube. The remaining cells were washed twice in ice cold PBS and then incubated on ice with protein extraction buffer (10 mM Tris-HCl, 150 mM NaCl, 1 mM EDTA, 1% Triton-X, 0.1% SDS and a protease inhibitor) for 20 min. Cells were sonicated and centrifuged for 5 min at 4° C. and the supernatant removed for subsequent analysis. Protein samples were stored at −70° C. until analyses. 8, 10 or 12% acrylamide gels were prepared and used depending on size of protein to be determined. Protein concentrations were determined using the BCA™ (bicinchoninic acid) protein determination assay (Pierce) according to manufacturer's guidelines and 30-50 μg protein samples were loaded and separated on the acrylamide gels. Gels were ran at 150V for approximately 2 h at 4° C. Following separation of the protein samples each was transferred to a PVDF transfer membrane (Hybond-P) overnight at 4° C. at 40V. Membranes were then washed in PBS and then blocked in 5% skimmed milk/0.05% Tween 20 for 1 h at room temperature or overnight at 4° C. Membranes were washed twice in PBS and incubated in primary antibody for either 1 h at room temperature or overnight at 4° C. The membranes were then further washed in PBS (3×5 min at room temperature) and then incubated in enzyme (HRP) conjugated secondary antibody for 1 h at room temperature. Additional washes were then carried out in PBS/0.05% Tween 20 followed finally by PBS alone. ECL plus (Amersham) or Supersignal® (Pierce) enzyme substrate systems were used to visualise protein bands according to manufacturer's guidelines.

In viva Human Xenograft Studies

To determine the antitumour effect of mellein on the growth of a human tumour in vivo MDA-MB-468 breast cancer cells (1×10⁶ cells per site) were injected subcutaneously into 6-7 week old female athymic nu/nu nude mice (Harlan). Tumour cells were injected suspended in 50:50 mix of Hanks buffered salt solution (HBSS) and matrigel (BD Biosciences). After a 3 week growth period animals were randomly allocated to treatment and control groups according to tumour volume. Animals received daily i.p. injections of either mellein (100 mg/ml) or a vehicle control. Tumour measurements were performed every 2-3 days using digital callipers and the tumour area and volume were calculated. The study was terminated after the animals mere on treatment for 46 days. On sacrifice tumours and major organs (heart, liver, lung, spleen and kidneys) were excised and fixed in formalin for subsequent pathological analysis. All animal studies were performed according to Home Office Guidelines and were approved by Queens University Animal Ethical Committee.
Mosmann, T. (1983) Rapid calorimetric assay for cellular growth and survival: application to proliferation and cytotoxicity assays. Journal of Immunological Methods 65: 55-63.

1. A compound for use in therapy, said compound being defined by formula I or a alpha pharmaceutically acceptable salt of Formula 1: 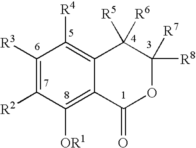
Formula 1
wherein
R¹ represents H, a C₁₋₂₅ aliphatic or aromatic hydrocarbon group CHO, COR⁹, CO₂R⁹, CONR₂⁹, SO₂R⁹, SO₃R⁹, PO(OR₉)₂, PO(NR₂⁹)₂ or PO(OR⁹)NR₂⁹;
R² to R⁸ are independently selected from the group consisting of H, a C₁₋₂₅ aliphatic or aromatic hydrocarbon group, OH, OR⁹, OCOR⁹, OSO₂R⁹, OPO(OR⁹)₂, OPO(OR⁹)NR₂⁹, OPO(NR₂⁹)₂, NH₂, NR₂⁹, COR⁹, SO₂R⁹, CN, NO₂, halogen, SO₃H, CHO, COR⁹, SH, SR⁹, SOR⁹, PO(OR⁹)₂, PO(OR⁹)NR₂⁹, CO₂H, CO₂R⁹, CONR₂⁹, SO₃R⁹, PO(NR₂⁹)₂, N₃ or SO₂NR₂⁹ wherein R⁵, R⁶ and/or R⁷ and R⁸ are not simultaneously OH, SH or NH₂ wherein R⁴ does not represent Cl; and each R⁹ group is independently selected from the group consisting of H, C_{1 to 25} alkyl, C_{1 to 25} alkenyl, C_{1 to 25} alkynyl, C_{6 to 14} aryl or C_{7 to 25} aralkyl.

2. The compound of claim 1 wherein:
R¹ represents H; and
R³ to R⁸ represent substituted or unsubstituted aliphatic or aromatic hydrocarbon group, H, OH, OR⁹, OCOR⁹, NH², NR₂⁹, COR⁹, SO₂R⁹, CN, NO₂, halogen, SO₃H, CHO, SH, SR⁹, SOR⁹, PO(OR⁹)₂, CO₂H, PO(OR⁹)NR₂⁹, PO(NR⁹)₂, CONR₂⁹, SO₂NR₂⁹ or N₃.

3. The compound of claim 1 wherein:
R¹ represents H;
R² represents H, F, I, 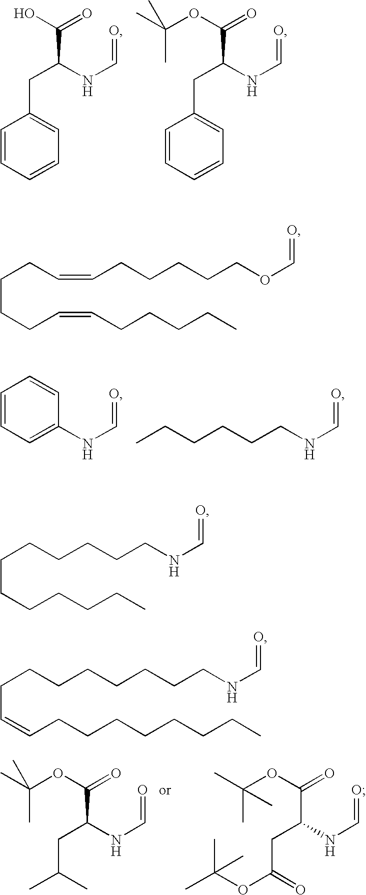
and
R⁷ and R⁸ represent CH₃ or H wherein one of both of R⁷ and R⁸ represent CH₃ or one or both of R⁷ and R⁸ represent H.

4. (canceled)

5. The compound of claim 1 having an IC 50 value of 1000 μM or less against cancer cell lines.

6. An anti-proliferative composition comprising the compound of claim 1 and a pharmaceutically acceptable excipient.

7. The anti-proliferative composition of claim 6 comprising one or more known cancer drugs.

8. The composition of claim 7 comprising an agent targeted against, microtubules, an agent targeted against topoisomerase enzymes or an agent that cross-links or damages DNA.

9. The composition of claim 7 comprising vinorelbine, irinotecan, cisplatin, etoposide, doxorubicin or docetaxel.

10. The composition of claim 6 formulated as a liquid preparation, a semisolid preparation, a solid oral preparation, a chewing gum preparation, an ear preparation, an eye preparation, a foam preparation, a granule preparation, an intramammary preparation, an intraruminal preparation, a liquid preparation, a semi-solid preparation, a solid cutaneous or transdermal preparation, nasal preparation, parenteral preparation, premix preparation for feeding stuffs, preparation for inhalation, preparation for irrigation, pressurised preparation, rectal preparation, subcutaneous preparation, tampon preparation, vaginal preparation, intravaginal preparation, implantable preparation, oromucosal preparation, preparation for dental use, tracheopulmonary preparation, preparation for dialysis, endocervical preparation, intrauterine preparation, or preparation for intravesical and urethral use.

11. (canceled)

12. (canceled)

13. A method of treating neoplasia comprising administering a composition comprising the compound of claim 1 to a patient.

14. A method of inducing apoptosis in a neoplastic cell comprising administering to the cell a composition comprising the compound of claim 1 in an amount sufficient to induce apoptosis.

15. An assay comprising
contacting a sample with a composition comprising the compound of claim 1
determining if said compound binds to a component of said sample; and
isolating a component which binds said compound.

16. A compound according to claim 1 wherein R² does not represent H and the compound is not ochratoxin B.

17. A compound according to claim 1 wherein the compound is not ALM-1, ALM-7 to ALM-5. ALM-24 or ALM-32 to ALM-34.

18. A compound as in claim 16 wherein:
R¹, R³. R⁴R⁵ and R⁶ represent H;
R⁷ and R⁸ represent H or CH₃ wherein one or both of R⁷ and R⁸ represent CH₃ or one or both of R⁷ and R⁸ represent H; and
R² represents F, I,

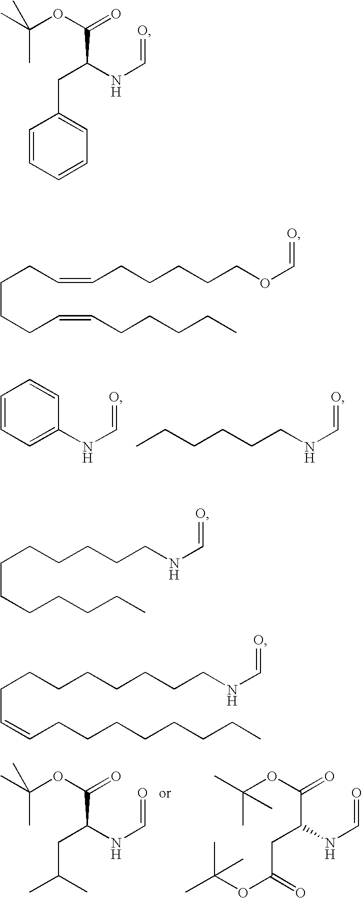

19. The method of claim 13, wherein the neoplasia is skin, breast, lung, prostate or colon cancer.
